(19) United States
(12) Patent Application Publication
(10) Pub. No.: US 2012/0252737 A1
(43) Pub. Date: Oct. 4, 2012

(54) Methods for Diagnosing and Treating Cancer

(75) Inventors: Michael B. Yaffee, West Roxbury, MA (US); Hans Christian Reinhardt, Koln (DE)

(73) Assignee: Massachusetts Institute of Technology, Cambridge, MA (US)

(21) Appl. No.: 13/499,785

(22) PCT Filed: Oct. 4, 2010
(86) PCT No.: PCT/US10/51326

§ 371 Date: Jun. 21, 2012

Related U.S. Application Data
(60) Provisional application No. 61/248,175, filed on Oct. 2, 2009.

(51) Int. Cl. A61K 38/02; G01N 21/64; C40B 30/04; A61K 31/713; C40B 40/06; C40B 40/10; A61P 35/00; G01N 33/574; C12Q 1/68

(52) U.S. Cl. 514/19.3; 435/7.23; 435/7.4; 506/9; 435/7.1; 435/6.14; 506/16; 506/18; 514/44 A

(57) ABSTRACT

The invention provides methods for decreasing one or more symptoms of cancer in a patient requiring the steps of determining the activation or inactivation of the MK2 signaling pathway and, based on these determinations, administering either a MK2 inhibitor or a combination of a MK2 inhibitor and a chemotherapeutic agent, or a chemotherapeutic agent to the patient. The invention further provides methods for identifying a cancer patient that may selectively benefit from the administration of a chemotherapeutic agent, or the administration of a MK2 inhibitor or the combination of a MK2 inhibitor and a chemotherapeutic agent, requiring the steps of determining the activation or inactivation of the MK2 signaling pathway. The invention additionally provides methods and kits for diagnosing a chemotherapy-sensitive or chemotherapy-resistant cancer in a subject that require the step of (or reagents for) determining the activation or inactivation of the MK2 signaling pathway. The invention also provides methods of treating a cancer patient diagnosed as having a chemotherapy-sensitive or a chemotherapy-resistant cancer.

### CROSS-REFERENCE TO RELATED APPLICATIONS

This application claims the benefit of the filing date of U.S. Provisional Application No. 61/248,175, filed Oct. 2, 2009, herein incorporated by reference in its entirety.

### FIELD OF THE INVENTION

The present invention relates to the field of cancer biology and molecular medicine.

### BACKGROUND OF THE INVENTION

The maintenance of genomic integrity is essential for the health of multi-cellular organisms. DNA damage checkpoints constitute a mechanism where cell division is delayed to allow repair of damaged DNA, or if the extent of DNA damage is beyond repair, induce apoptosis. The three major DNA damage-responsive cell cycle checkpoints are the G₁/S checkpoint, intra S-phase checkpoint, and the G₂/M checkpoint.

In response to DNA damage, eukaryotic cells activate a complex signaling network to arrest the cell cycle and facilitate DNA repair. This signaling network has traditionally been divided into two major protein kinase pathways, one mediated by Ataxia-Telangiectasia mutated (ATM) through Chk2, and the other mediated by Ataxia-Telangiectasia and Rad-3 related (ATR) through Chk1. Some cross-talk exists between the ATM/Chk2 and ATR/Chk1 kinase pathways, particularly when signaling through one pathway is partially or totally deficient. Normally, however, the pathways show only partial functional overlap in response to particular forms of DNA damage. The ATM/Chk2 pathway responds primarily to DNA double strand breaks (DSBs), while the ATR/Chk1 pathway is activated by bulky DNA lesions, and following replication fork collapse during S-phase. The tumor suppressor protein p53 is a major downstream effector of these DNA damage kinase pathways. In normal cells, p53-dependent signaling results in G₁ arrest, mainly mediated by transcriptional upregulation of p21. In addition, p21 also appears to play a role in sustaining the G₂ checkpoint after γ-irradiation. If the DNA damage is extensive, however, then p53-dependent pathways target the damaged cell for apoptotic cell death through both the intrinsic and extrinsic pathways. Most tumor cells show specific disruptions in the p53 pathway, leading to selective loss of the G₁ checkpoint. These cells are then entirely dependent on intra-S and G₂/M checkpoints to maintain their genomic integrity in response to DNA damage.

A third checkpoint effector pathway mediated by p38 and MAPKAP kinase-2 (MK2) that operates parallel to Chk1 and is activated downstream of ATM and ATR has been identified. This p38/MK2 pathway is a global stress-response pathway which, in response to genotoxic stress, becomes co-opted as part of the ATM/ATR-dependent cell-cycle checkpoint machinery. In particular, it is specifically within cells defective in the ARF/p53 pathway that cannot induce high levels of the Cdk inhibitor p21 that the p38/MK2 pathway becomes essential for proper cell-cycle control following DNA damage, despite a functional ATR-Chk1 pathway.

As noted above, the activation of cell cycle checkpoint pathways is critical to the survival of cells following genotoxic exposures, such as the survival of cancer cells following exposure to DNA-damaging chemotherapeutic agents. In view of the need for further cancer therapies, the invention provides methods of treating and diagnosing cancer that require the analysis of the activation or inactivation of the p38/MK2 signaling pathway.

### SUMMARY OF THE INVENTION

Applicants have characterized the p38/MK2 signaling pathway (MK2 signaling pathway) that is activated in response to genotoxic stress (e.g., DNA damage) and have discovered that this pathway is critical for prolonged (late) G₂/M checkpoint maintenance in a subset of cancers, particularly those that are deficient in p53-dependent responses. In view of this discovery, the present invention provides methods of evaluating the activation or inactivation of the MK2 pathway in cancer cell(s) from a patient and utilizing this information to provide effective methods for identifying a cancer patient that may selectively benefit from the administration of one or more MK2 inhibitor(s), one or more chemotherapeutic agent(s), or a combination of one or more MK2 inhibitor(s) and one or more chemotherapeutic agent(s), and methods of diagnosing a chemotherapy-resistant or chemotherapy-sensitive cancer in a patient. The invention also provides kits for diagnosing a chemotherapy-resistant or chemotherapy-sensitive cancer in a patient containing reagents that are capable of measuring one or more marker(s) of MK2 signaling pathway activation or inactivation and p53 signaling pathway inactivation, and instructions for using these reagents.

The invention provides methods of reducing the severity of one or more symptom(s) of cancer in a patient requiring the steps of (i) measuring one or more feature(s) (e.g., at least two) in a cancer cell(s) from the patient selected from the group of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; (ii) determining from the measurements in step (i) whether the cancer cell(s) in the patient has one or more feature(s) of an activated MK2 signaling pathway selected from the group of: increased cytoplasmic MK2 protein localization, decreased nuclear MK2 protein localization, increased phosphorylation of total MK2 protein, increased levels of phosphorylated MK2 protein in the cytoplasm or nucleus, increased levels of phosphorylated hsp27, increased levels of phosphorylated hnRNPA0, increased levels of phosphorylated PARN, increased levels of phosphorylated TIAR, increased levels of phosphorylated cdc25B, increased levels of phosphorylated cdc25C, and increased levels of Gadd45a protein or mRNA relative to these features in a control sample; and (iii) administering to a patient determined to have a cancer cell having one or more the feature(s) of an activated MK2 signaling pathway one or more MK2 inhibitor(s) for a time and in an amount sufficient to reduce the severity of one or more symptom(s) of cancer in the patient.

Additional embodiments of the above methods include the further steps of: (iv) measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; (v) determining from the measurements in step (iv) whether the cancer cell(s) in the patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; and (vi) administering to a patient determined to have a cancer cell having one or more the feature(s) of an activated MK2 signaling pathway and one or more the feature(s) of a defective p53 pathway one or more MK2 inhibitor(s) for a time and in an amount sufficient to reduce the severity of one or more symptom(s) of cancer in the patient. Further embodiments of any of the above methods further comprise the step of administering one or more chemotherapeutic agent(s) (e.g., a chemotherapeutic agent that induces DNA damage) to the patient.

In any of the above methods, the control sample in step (ii) may be a non-cancerous cell or a cell untreated with a genotoxic agent and/or the control sample in step (v) is a non-cancerous cell.

In any of the above methods, the MK2 inhibitor may be a small molecule. For example, the MK2 inhibitor may be a small molecule selected from the group of: UCN-01, 2-(3-aminopropyl)-8-(methylthio)-2,4,5,6-tetrahydropyrazolo[3,4-e]indazole-3-carboxylic acid dihydrochloride, 2-(3-aminopropyl)-2,4,5,6-tetrahydropyrazolo[3,4-e]indazole-3-carboxylic acid hydrochloride, 2-(3-{[2-(4-bromophenyl)ethyl]amino}propyl)-2,4,5,6-tetrahydropyrazolo[3,4-e]indazole-3-carboxylic acid hydrochloride, 2-(2-aminoethyl)-2,4,5,6-tetrahydropyrazolo[3,4-e]indazole-3-carboxylic acid hydrochloride, 8-(allylthio)-2-(3-aminopropyl)-2,4,5,6-tetrahydropyrazolo[3,4-e]indazole-3-carboxylic acid dihydrochloride, 2-(3-aminopropyl)-8-(benzylthio)-2,4,5,6-tetrahydropyrazolo[3,4-e]indazole-3-carboxylic acid dihydrochloride, 2-{3-[(2-thien-2-ylethyl)amino]propyl}-2,4,5,6-tetrahydropyrazolo[3,4-e]indazole-3-carboxylic acid, 2-{3-[(2-thien-3-ylethyl)amino]propyl}-2,4,5,6-tetrahydropyrazolo[3,4-e]indazole-3-carboxylic acid hydrochloride, ethyl 2-(3-{[2-(4-bromophenyl)ethyl]amino}propyl)-2,4,5,6-tetrahydropyrazolo[3,4-e]indazole-3-carboxylate, 2-(3-aminopropyl)-8-quinolin-3-yl-4,5-dihydro-2H-pyrazolo[4,3-h]quinazoline-3-carboxylic acid dihydrochloride, 2-(3-aminopropyl)-8-(1,3-benzodioxol-5-yl)-4,5-dihydro-2H-pyrazolo[4,3-h]quinazoline-3-carboxylic acid hydrochloride, 2-(3-aminopropyl)-8-phenyl-4,5-dihydro-2H-pyrazolo[4,3-h]quinazoline-3-carboxylic acid hydrochloride, 2-quinolin-3-yl-5,6,8,9,10,11-hexahydro-7H-[1,4]diazepino[1′,2′:1,5]pyrazolo[4,3-h]quinazolin-7-one, 2-pyridin-3-yl-5,6,8,9,10,11-hexahydro-7H-(1,4]diazepino[1′,2′:1,5]pyrazolo[4,3-h]quinazolin-7-one, 8-quinolin-3-yl-2-[3-(tritylamino)propyl]-4,5-dihydro-2H-pyrazolo[4,3-h]quinazoline-3-carboxylic acid hydrochloride, 2-(1,3-benzodioxol-5-yl)-5,6,8,9,10,11-hexahydro-7H-[1,4]diazepino[1′,2′:1,5]pyrazolo[4,3-h]quinazolin-7-one, 2-(4-methoxyphenyl)-5,6,8,9,10,11-hexahydro-7H-[1,4]diazepino[1′,2′:1,5]pyrazolo[4,3-h]quinazolin-7-one, 2-pyridin-4-yl-5,6,8,9,10,11-hexahydro-7H-[1,4]diazepino[1′,2′:1,5]pyrazolo[4,3-h]quinazolin-7-one hydrochloride, 2-(3-aminopropyl)-8-quinolin-3-yl-4,5-dihydro-2H-pyrazolo[3,4-f]isoquinoline-3-carboxylic acid trifluoroacetate, 2-(3-aminopropyl)-8-(3-nitrophenyl)-4,5-dihydro-2H-pyra-zolo[3,4-f]isoquinoline-3-carboxylic acid trifluoroacetate, 2-(3-aminopropyl)-8-(4-hydroxyphenyl)-4,5-dihydro-2H-pyrazolo[3,4-f]isoquinoline-3-carboxylic acid hydrobromide, 2-(3-aminopropyl)-8-(3-hydroxyphenyl)-4,5-dihydro-2H-pyrazolo[3,4-f]isoquinoline-3-carboxylic acid hydrobromide, 2-(3-aminopropyl)-8-(2-naphthyl)-4,5-dihydro-2H-pyrazolo[3,-4-f]isoquinoline-3-carboxylic acid trifluoroacetate, 2-(3-aminopropyl)-8-(3,5-difluorophenyl)-4,5-dihydro-2H-pyrazolo[3,4-f]isoquinoline-3-carboxylic acid trifluoroacetate, 2-(3-aminopropyl)-8-(1,3-benzodioxol-5-yl)-4,5-dihydro-2H-pyrazolo[3,4-q]isoquinoline-3-carboxylic acid trifluoroacetate, 2-(3-aminopropyl)-8-(3-cyanophenyl)-4,5-dihydro-2H-pyrazolo[3,4-q]isoquinoline-3-carboxylic acid trifluoroacetate, 9-(hydroxymethyl)-2-quinolin-3-yl-5,6,9,10-tetrahydropyrazino[1′,2′:1,5]pyrazolo[3,4-f]isoquinolin-7(8H)-one trifluoroacetate, 2-(3-aminopropyl)-8-(4-methoxyphenyl)-4,5-dihydro-2H-pyrazolo[3,4-q]isoquinoline-3-carboxylic acid trifluoroacetate, 2-(3-aminopropyl)-8-[3-(methyl-sulfonyl)phenyl]-4,5-dihydro-2H-pyrazolo[3,4-q]isoquinoline-3-carboxylic acid trifluoroacetate, 2-(3-aminopropyl)-8-[3-(trifluoromethyl)phenyl]-4,5-dihydro-2H-pyrazolo[3,4-q]isoquinoline-3-carboxylic acid trifluoroacetate, 2-(1H-imidazol-1-yl)-5,6,9,10-tetrahydropyrazino[1′,2′:1,5]pyrazolo[3,4-q]isoquinolin-7(8H)-one trifluoroacetate, 2-(3-aminopropyl)-8-(3-methoxyphenyl)-4,5-dihydro-2′-1-pyrazolo[3,4-f]isoquinoline-3-carboxylic acid trifluoroacetate, 2-(3-aminopropyl)-8-[4-(trifluoromethyl)phenyl]-4,5-dihydro-2H-pyrazolo[3,4-f]isoquinoline-3-carboxylic acid trifluoroacetate, 2-(3-aminopropyl)-8-anilino-4,5-dihydro-2H-pyrazolo[3,4-f]isoquinoline-3-carboxylic acid, 2-(3-aminopropyl)-8-(3,4-difluorop-henyl)-4,5-dihydro-2H-pyrazolo[3,4-f]isoquinoline-3-carboxylic acid trifluoroacetate, 2-(3-{[2-(4′-carboxy-1,1′-biphenyl-4-yl)ethyl]amino}propyl)-4,5-dihydro-2H-pyrazolo[3,4-f]isoquinoline-3-carboxylic acid trifluoroacetate, 2-(4-hydroxyphenyl)-5,6,9,10-tetrahydropyrazino[1′,2′: 1,5]pyrazolo[3,4-f]isoquinolin-7(8H)-one trifluoroacetate, 2-propyl-8-quinolin-3-yl-4,5-dihydro-2H-pyrazolo[3,4-f]isoquinoline-3-carboxylic acid, 2-[3-({2-[3′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate, 2-(3-aminopropyl)-8-(4-tert-butylphenyl)-4,5-dihydro-2H-pyrazolo[3,4-f]isoquinoline-3-carboxylic acid trifluoroacetate, 2-[3-({2-[4-(3-furyl)phenyl]ethyl}amino) propyl]-4,5-dihydro-2H-pyrazolo[3,4-f]isoquinoline-3-carboxylic acid trifluoroacetate, 2-(3-{[2-(3′-chloro-1,1′-biphenyl-4-yl)ethyl]amino}propyl)-4,5-dihydro-2H-pyrazolo[3,4-f]isoquinoline-3-carboxylic acid trifluoroacetate, 2-(4-methoxyphenyl)-5,6,8,9,10,11-hexahydro-7H-[1,4]diazepino[1′,2′:1,5]pyrazolo[3,4-f]isoquinolin-7-one, 2-[3-({2-[4′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate, 2-(3-hydroxyphenyl)-5,6,9,10-tetrahydropyrazino[1′,2′-:1,5]pyrazolo[3,4-f]isoquinolin-7(8H)-one, 2-(3-aminopropyl)-N-hydroxy-8-quinolin-3-yl-4,5-dihydro-2H-pyrazolo[3,4-f-]isoquinoline-3-carboxamide hydrochloride, 2-[(E)-2-(4-hydroxyphenyl)ethenyl]-5,6,9,10-tetrahydropyrazino[1′,2′:1,5]pyrazolo[3,4-f]isoquinolin-7(8H)-one trifluoroacetate, 2-quinolin-3-yl-5,6,8,9,10,11-hexahydro-7H-[1,4]diazepino[1′,2′: 1,5]pyrazolo[3,4-f]isoquinolin-7-one, 2-(4-hydroxyphenyl)-5,6,8,9,10,11-hexahydro-7H-[1,4]diazepino[1′,2′:1,5]pyrazolo[3,4-f]isoquinolin-7-one, 2-(3-{[2-(2′-chloro-1,1′-biphenyl-4-yl)ethyl]amino}propyl)-4,5-dihydro-2H-pyrazolo[3,4-f]isoquinoline-3-carboxylic acid trifluoroacetate, 2-(3-{[2-(4′-tert-butyl-1,1′-biphenyl-4-yl)ethyl]amino}propyl)-4,5-dihydro-2H-pyrazolo[3,4-f]isoquinoline-3-carboxylic acid trifluoroacetate, 2-(3-{[2-(3,4-dichlorophenyl)ethyl]amino}propyl)-4,5-dihydro-2H-pyrazolo[3,4-f]isoquinoline-3-carboxylic acid trifluoroacetate, 2-(3-{[3-(3-chlorophenyl)propyl]amino}propyl)-4,5-dihydro-2H-pyrazolo[3,4-f]isoquinoline-3-carboxylic acid trifluoroacetate, 2-[(E)-2-phenylethenyl]-5,6,9,10-tetrahydropyrazino[1′,2′:1,5]pyrazolo[3,4-f]isoquinolin-7(8H)-one trifluoroacetate, 2-(3-{[2-(4-pyridin-4-ylphenyl)ethyl]amino}propyl)-4,5-dihydro-2H-pyrazolo[3,4-f]isoquinoline-3-carboxylic acid trifluoroacetate, 2-(3-{[3-(4-bromophenyl)propyl]amino}propyl)-4,5-dihydro-2H-pyrazolo[3,4-f]isoquinoline-3-carboxylic acid trifluoroacetate, 2-(3-{[3-(4-tert-butylphenyl)propyl]amino}propyl)-4,5-dihydro-2H-pyrazolo[3,4-f]isoquinoline-3-carboxylic acid trifluoroacetate, 2-(3-{[2-(3′-isopropyl-1,1′-biphenyl-4-yl)ethyl]amino}propyl)-4,5-dihydro-2H-pyrazolo[3,4-f]isoquinoline-3-carboxylic acid trifluoroacetate, 2-{3-[(2-thien-2-ylethyl)amino]propyl}-4,5-dihydro-2H-pyrazolo[3,4-f] isoquinoline-3-carboxylic acid trifluoroacetate, 2-[4-(dimethylamino)phenyl]-5,6,9,10-tetrahydropyrazino[1′,2′:1,5]pyrazolo[3,4-f]isoquinolin-7(8H)-one, 2-(3-{[2-(1,1′-biphenyl-4-yl)ethyl]amino}propyl)-4,5-dihydro-2H-pyrazolo[3,4-f]isoquinoline-3-carboxylic acid trifluoroacetate, 2-(3-methoxyphenyl)-5,6,9,10-tetrahydropyrazino[1′,2′: 1,5]pyrazolo[3,4-f]-isoquinolin-7(8H)-one, 2-(3-{[2-(4-bromophenyl)ethyl]amino}propyl)-4,5-dihydro-2H-pyrazolo[3,4-f]isoquinoline-3-carboxylic acid trifluoroacetate, 2-(3-{[2-(2,4-dichlorophenyl)ethyl]amino}propyl)-4,5-dihydro-2H-pyrazolo[3,4-f]isoquinoline-3-carboxylic acid trifluoroacetate, 2-{3-[(benzylsulfonyl)amino]propyl}-8-quinolin-3-yl-4,5-dihydro-2H-pyrazolo[3,4-f]isoquinoline-3-carboxylic acid, 9-(aminomethyl)-5,6,9,10-tetrahydropyrazino[1′,2′:1,5]pyrazolo[3,4-f]isoquinolin-7(8H)-one tritluoroacetate, 2-(3-nitrophenyl)-5,6,8,9,10,11-hexahydro-7H-[1,4]diazepino[1′,2′:1,5]pyrazolo[3,4-f]isoquinolin-7-one, 2-(3-aminopropyl)-8-quinolin-3-yl-4,5-dihydro-2H-pyrazolo[3,4-f]isoquinoline-3-carboxamide hydrochloride, 2-(3-{[3-(4-chlorophenyl)propyl]amino}propyl)-4,5-dihydro-2H-pyrazolo[3,4-f]isoquinoline-3-carboxylic acid trifluoroacetate, 2-[3-(dimethylamino)phenyl]-5,6,8,9,10,11-hexahydro-7H-[1,4]diazepino[1′,2′:1,5]pyrazolo[3,4-f]isoquinolin-7-one, 2-(3-{[(4-chlorobenzyl)sulfonyl]amino}propyl)-8-quinolin-3-yl-4,5-dihydro-2H-pyrazolo[3,4-f]isoquinoline-3-carboxylic acid, 2-(3-{[2-(4-pyridin-3-ylphenyl)ethyl]amino}propyl)-4,5-dihydro-2H-pyrazolo[3,4-f]isoquinoline-3-carboxylic acid trifluoroacetate, 2-(3-{[2-(4-chlorophenyl)ethyl]amino}propyl)-4,5-dihydro-2H-pyrazolo[3,4-f]isoquinoline-3-carboxylic acid trifluoroacetate, 2-(3-{[2-(5-chlorothien-2-yl)ethyl]amino}propyl)-4,5-dihydro-2H-pyrazolo[3,4-f]isoquinoline-3-carboxylic acid trifluoroacetate, 2-(3-{[3-(4-cyanophenyl)propyl]amino}propyl)-4,5-dihydro-2H-pyrazolo[3,4-f]isoquinoline-3-carboxylic acid trifluoroacetate, 2-(3-{[3-(5-methyl-2-furyl)butyl]amino}propyl)-4,5-dihydro-2H-pyrazolo[3,4-f]isoquinoline-3-carboxylic acid trifluoroacetate, 2-[3-({2-[4-(1-benzothien-3-yl)phenyl]ethyl}amino)propyl]-4,5-dihydro-2H-pyrazolo[3,4-f]isoquinoline-3-carboxylic acid trifluoroacetate, 2-(3-ammoniopropyl)-3-carboxy-4,5-dihydro-2H-pyrazolo[3,4-f]isoquinolin-7-ium dichloride, 2-(4-methoxyphenyl)-5,6,9,10-tetrahydropyrazino[1′,2′:1,5]pyrazolo[3,4-f]isoquinolin-7(8H)-one, 2-(3-{[3-(4-acetylphenyl)propyl]amino}propyl)-4,5-dihydro-2H-pyrazolo[3,4-f]isoquinoline-3-carboxylic acid trifluoroacetate, 2-(3-aminopropyl)-4,5-dihydro-2H-pyrazolo[3,4-f]isoquinoline-3-carboxylic acid trifluoroacetate, 2-[3-({3-[4-(methylsulfonyl)phenyl]propyl}amino)propyl]-4,5-dihydro-2H-pyrazolo[3,4-f]isoquinoline-3-carboxylic acid trifluoroacetate, 2-(3-aminopropyl)-8-(2-methoxyphenyl)-4,5-dihydro-2H-pyrazolo[3,4-f]isoquinoline-3-carboxylic acid trifluoroacetate, 2-[3-({2-[3′-(aminomethyl)-1,1′-biphenyl-4-yl]ethyl}amino)propyl]-4,5-dihydro-2H-pyrazolo[3,4-f]isoquinoline-3-carboxylic acid trifluoroacetate, 2-(2-aminoethyl)-4,5-dihydro-2H-pyrazolo[3,4-f]isoquinoline-3-carboxylic acid dihydrochloride, 2-(3-{[2-(4-nitrophenyl)ethyl]amino}propyl)-4,5-dihydro-2H-pyrazolo[3,4-f]isoquinoline-3-carboxylic acid trifluoroacetate, 2-[3-({2-[2′-(trifluoromethyl)-1,1′-biphenyl-4-yl]ethyl}amino)propyl]-4,5-dihydro-2H-pyrazolo[3,4-f]isoquinoline-3-carboxylic acid trifluoroacetate, 9-(hydroxymethyl)-5,6,9,10-tetrahydropyrazino[1′,2′:1,5]pyrazolo[3,4-f]isoquinolin-7(8H)-one, 243-{[2-(4-methylphenyl)ethyl]amino}propyl)-4,5-dihydro-2H-pyrazolo[3,4-f]isoquinoline-3-carboxylic acid trifluoroacetate, 9-{[(2-thien-2-ylethyl)amino]methyl}-5,6,9,10-tetrahydropyrazino[1′,2′:1,5]pyrazolo[3,4-f]isoquinolin-7(8H)-one, 2-(3-{[2-(4-ethoxyphenyl)ethyl]amino}propyl)-4,5-dihydro-2H-pyrazolo[3,4-f]isoquinoline-3-carboxylic acid trifluoroacetate, 2-(1,3-benzodioxol-5-yl)-5,6,8,9,10,11-hexahydro-7H-[1,4]diazepino[1′,2′: 1,5]pyrazolo[3,4-f]isoquinolin-7-one, 2-(3-{[2-(4-methoxyphenyl) ethyl]amino}propyl)-4,5-dihydro-2H-pyrazolo[3,4-f]isoquinoline-3-carboxylic acid trifluoroacetate, 3-[3-(1H-tetraazol-5-yl)-4,5-dihydro-2H-pyrazolo[3,4-f]isoquinolin-2-yl]propan-1-amine hydrochloride, 2-(3-aminopropyl)-8-chloro-4,5-dihydro-2H-pyrazolo[3,4-q]isoquinoline-3-carboxylic acid trifluoroacetate, 2-(3-{[(1R,2S)-2-phenylcyclopropyl]amino}propyl)-4,5-dihydro-2H-pyrazolo[3,4-f]isoquinoline-3-carboxylic acid trifluoroacetate, 2-{3-[(3,3-diphenylpropyl)amino]propyl}-4,5-dihydro-2H-pyrazolo[3,4-f]isoquinoline-3-carboxylic acid trifluoroacetate, 2-(3-{[2-(3-bromo-4-methoxyphenyl)ethyl]amino}propyl)-4,5-dihydro-2H-pyrazolo[3,4-f]isoquinoline-3-car-boxylic acid trifluoroacetate, 2-{3-[(4-phenylbutyl)amino]propyl}-4,5-dihydro-2H-pyrazolo[3,4-f]isoquinoline-3-carboxylic acid trifluoroacetate, 2-[3-(2,3-dihydro-1H-inden-2-ylamino)propyl]-4,5-dihydro-2H-pyrazolo[3,4-f]isoquinoline-3-carboxylic acid trifluoroacetate, 2-(2-naphthyl)-5,6,8,9,-10,11-hexahydro-7H-[1,4]diazepino[1′,2′:1,5]pyrazolo[3,4-f]isoquinolin-7-one, 2-{3-[(3-phenylpropyl)amino]propyl}-4,5-dihydro-2H-pyrazolo[3,4-f]isoquinoline-3-carboxylic acid trifluoroacetate, 2-(3-{[2-(4-fluorophenyl)ethyl]amino}propyl)-4,5-dihydro-2H-pyrazolo[3,4-f]isoquinoline-3-carboxylic acid trifluoroacetate, 2-{3-[(2-thien-3-ylethyl)amino]propyl}-4,5-dihydro-2H-pyrazolo[3,4-f]isoquinoline-3-carboxylic acid dihydrochloride, 5,6,9,10-tetrahydropyrazino[1′,2′:1,5]pyrazolo[3,4-f]isoquinolin-7(8H)-one, 2-[3-(glycoloylamino) propyl]-8-quinolin-3-yl-4,5-dihydro-2H-pyrazolo[3,4-f]isoquinoline-3-carboxylic acid hydrochloride, 8-quinolin-3-yl-4,5-dihydro-2H-pyrazolo[3,4-f]isoquinoline-3-carboxylic acid, 2-(3-{[2-(4-ethylphenyl)ethyl]amino}propyl)-4,5-dihydro-2H-pyrazolo[3,4-f]isoquinoline-3-carboxylic acid trifluoroacetate, 2-(3-{[2-(2-chlorophenyl)ethyl]amino}propyl)-4,5-dihydro-2H-pyrazolo[3,4-f]isoquinoline-3-carboxylic acid trifluoroacetate, 2-{3-[(2-ethylbutyl)amino]propyl}-4,5-dihydro-2H-pyrazolo[3,4-f]isoquinoline-3-carboxylic acid trifluoroacetate, 8-quinolin-3-yl-4,5-dihydro-2H-pyrazolo[3,4-f]isoquinoline-3-carboxamide, 2-{3-[(2-pyridin-4-ylethyl)amino]propyl}-4,5-dihydro-2H-pyrazolo[3,4-f]isoquinoline-3-carboxylic acid trifluoroacetate, 2-(3-{[2-(4-chlorophenyl)propyl]amino}propyl)-4,5-dihydro-2H-pyrazolo[3,4-f]isoquinoline-3-carboxylic acid trifluoroacetate, 2-(3-{[2-(3-chlorophenyl)ethyl]amino}propyl)-4,5-dihydro-2H-pyrazolo[3,4-f]isoquinoline-3-carboxylic acid trifluoroacetate, 2-[3-(glycoloylamino)propyl]-4,5-dihydro-2H-pyrazolo[3,4-f]isoquinoline-3-carboxylic acid trifluoroacetate, 2-(3-{[2-(3,4-dimethoxyphenyl)ethyl]amino}propyl)-4,5-dihydro-2H-pyrazolo[3,4-f]isoquinoline-3-carboxylic acid trifluoroacetate, 2-(1-benzofuran-2-yl)-5,6,8,9,10,1,1-hexahydro-7H-[1,4]diazepino[1′,2′:1,5]pyrazolo[3,4-f]isoquinolin-7-one, 2-(3-{[4-(2-aminoethyl)phenyl]amino}propyl)-4,5-dihydro-2H-pyrazolo[3,4-f]isoquinoline-3-carboxylic acid trifluoroacetate, 2-(1-naphthyl)-5,6,9,10-tetrahydropyrazino[1′,2′:1,5]pyrazolo[3,4-f]isoquinolin-7(8H)-one, 8-(3-aminopropyl)-5,6,8,9,10,11-hexahydro-7H-[1,4]diazepino[1′,2′:1,5]pyrazolo[3,4-f]isoquinolin-7-one dihydrochloride, 2-anilino-5,6,9,10-tetrahydropyrazino[1′,2′:1,5]pyrazolo[3,4-f]isoquinolin-7(8H)-one, 2-(3-aminopropyl)-8-[2-(trifluoromethyl)phenyl]-4,5-dihydro-2H-pyrazolo[3,4-f]isoquinoline-3-carboxylic acid trifluoroacetate, 9-(azidomethyl)-5,6,9,10-tetrahydropyrazino[1′,2′:1,5]pyrazolo[3,4-f]isoquinolin-7(8H)-one, 9-({[2-(4-chlorophenyl)ethyl]amino}methyl)-5,6,9,10-tetrahydropyrazino[1′,2′:1,5]pyrazolo[3,4-f]isoquinolin-7(8H)-one, 2-phenyl-5,6,8,9,10,11-hexahydro-7H-[1,4]diazepino[1′,2′:1,5]pyrazolo[3,4-f]isoquinolin-7-one, 2-[3-(pentylamino)propyl]-4,5-dihydro-2H-pyrazolo[3,4-f]isoquinoline-3-carboxylic acid trifluoroacetate, 10-(2-aminoethyl)-5,6,9,10-tetrahydropyrazino[1′,2′:1,5]pyrazolo[3,4-f]isoquinolin-7(8H)-one, 2-[3-(allylamino)propyl]-4,5-dihydro-2H-pyrazolo[3,4-f]isoquinoline-3-carboxylic acid trifluoroacetate, 2-(4-aminobutyl)-4,5-dihydro-2H-pyrazolo[3,4-f]isoquinoline-3-carboxylic acid dihydrochloride, 2-(3-{[2-(4-aminophenyl)ethyl]amino)propyl}-4,5-dihydro-2H-pyrazolo[3,4-f]isoquinoline-3-carboxylic acid trifluoroacetate, 2-[(1E)-3,3-dimethylbut-1-enyl]-5,6,9,10-tetrahydropyrazino[1′,2′:1,5]pyrazolo[3,4-f]isoquinolin-7-(8H)-one-trifluoroacetate, 10-(nitromethyl)-5,6,9,10-tetrahydropyrazino[1′,2′:1,5]pyrazolo[3,4-f]isoquinolin-7(8H)-one, 3-carboxy-2-[3-(methylammonio)propyl]-4,5-dihydro-2H-pyrazolo[3,4-f]isoquinolin-7-ium dichloride, 2-[3-({[(4-butoxyphenyl)amino]carbonyl}amino)propyl]-4,5-dihydro-2H-pyrazolo[3,4-f]isoquinoline-3-carboxylic acid, 2-{3-[(2-pyridin-3-ylethyl)amino]propyl}-4,5-dihydro-2H-pyrazolo[3,4-f]isoquinoline-3-carboxylic acid trifluoroacetate, 2-{3-[(2-pyridin-2-ylethyl)amino]propyl}-4,5-dihydro-2H-pyrazolo[3,4-f]isoquinoline-3-carboxylic acid trifluoroacetate, 2-{3-[(cyclopropylmethyl)amino]propyl}-4,5-dihydro-2H-pyrazolo[3,4-f]isoquinoline-3-carboxylic acid trifluoroacetate, 2-{3-[(2-thien-2-ylpropyl)amino]propyl}-4,5-dihydro-2H-pyrazolo[3,4-f]isoquinoline-3-carboxylic acid trifluoroacetate, 2-methoxy-5,6,9,10-tetrahydropyrazino[1′,2′:1,5]pyrazolo[3,4-f]isoquinolin-7(8H)-one, 2-[3-(dimethylamino)phenyl]-5,6,9,10-tetrahydropyrazino[1′,2′:1,5]pyrazolo[3,4-q]isoquinolin-7(8H)-one, 2-(3-{[2-(1H-pyrrol-1-yl)ethyl]amino}propyl)-4,5-dihydro-2H-pyrazolo[3,4-f]isoquinoline-3-carboxylic acid trifluoroacetate, 2-[3-(benzyloxy)propyl]-8-quinolin-3-yl-4,5-dihydro-2H-pyrazolo[3,4-f]isoquinoline-3-carboxylic acid, 2-{3-[(4-butoxybenzyl)amino]propyl}-4,5-dihydro-2H-pyrazolo[3,4-f]isoquinoline-3-carboxylic acid trifluoroacetate, 2-(1,3-benzodioxol-5-yl)-5-,6,9,10-tetrahydropyrazino[1′,2′:1,5]pyrazolo[3,4-f]isoquinolin-7(8H)-one, 2-[(E)-2-(2-fluorophenyl)ethenyl]-5,6,9,10-tetrahydropyrazino[1′,2′:1,5]pyrazolo[3,4-q]isoquinolin-7(8H)-one, 2-[(1E)-hex-1-enyl]-5,6,9,10-tetrahydropyrazino[1′,2′:1,5]pyrazolo[3,4-f]isoquinolin-7(8H)-one trifluoroacetate, 2-anilino-5,6,8,9,10,11-hexahydro-7H-[1,4]diazepino[1′,2′:1,5]pyrazolo[3,4-f]isoquinolin-7-one, 5,6,8,9,10,11-hexahydro-7H-[1,4]diazepino[1′,2′:1,5]pyrazolo[3,4-f]isoquinolin-7-one, 2-chloro-5,6,9,10-tetrahydropyrazino[1′,2′:1,5]pyrazolo[3,4-f]isoquinolin-7(8H)-one, 2-[(E)-2-(4-methoxyphenyl)ethenyl]-5,6,9,10-tetrahydropyrazino[1′,2′:1,5]pyrazolo[3,4-f]isoquinolin-7(8H)-one, 2-(methylthio)-5,6,9,10-tetrahydropyrazino[1′,2′:1,5]pyrazolo[3,4-f]isoquinolin-7(8H)-one, 2-{3-[(2-furylmethyl)amino]propyl}-4,5-dihydro-2H-pyrazolo[3,4-f]isoquinoline-3-carboxylic acid trifluoroacetate, 2-azepan-1-yl-5,6,9,10-tetrahydropyrazino[1′,2′:1,5]pyrazolo[3,4-f]isoquinolin-7(8H)-one trifluoroacetate, 2-(3,6-dihydropyridin-1(2H)-yl)-5,6,9,10-tetrahydropyrazino[1′,2′:1,5]pyrazolo[3,4-f]isoquinolin-7(8H)-one trifluoroacetate, 9-methyl-5,6,9,10-tetrahydropyrazino[1′,2′: 1,5]pyrazolo[3,4-f]isoquinolin-7(8H)-one, 2-(piperidin-3-ylmethyl)-4,5-dihydro-2H-pyrazolo[3,4-f]isoquinoline-3-carboxylic acid hydrochloride, 9-(chloromethyl)-5,6,9,10-tetrahydropyrazino[1′,2′: 1,5]pyrazolo[3,4-f]isoquinolin-7(8H)-one, 2-[(4-methoxybenzyl)amino]-5,6,9,10-tetrahydropyrazino[1′,2′:1,5]pyrazolo[3,4-f]isoquinolin-7(8H)-one trifluoroacetate, 2-(3-([2-(1H-imidazol-4-yl)-ethyl]amino}propyl)-4,5-dihydro-2H-pyrazolo[3,4-f]isoquinoline-3-carboxyli-c acid trifluoroacetate, 2-(benzylamino)-5,6,9,10-tetrahydropyrazino[1′,2′:1,5]pyrazolo[3,4-f]isoquinolin-7(8H)-one trifluoroacetate, 2-(methylthio)-5,6,8,9,10,11-hexahydro-7H-[1,4]diazepino[1′,2′:1,5]pyrazolo[3,4-f]isoquinolin-7-one, 2-{3-[(2-chlorobenzyl)amino]propyl}-4,5-dihydro-2H-pyrazolo[3,4-f]isoquinoline-3-carboxylic acid trifluoroacetate, 2-[3-(benzylamino)propyl]-4,5-dihydro-2H-pyrazolo[3,4-f]isoquinoline-3-carboxylic acid trifluoroacetate; 2-[3-({[(4-methoxyphenyl) amino]carbonyl}amino)propyl]-4,5-dihydro-2H-pyrazolo[3,4-f]isoquinoline-3-carboxylic acid, 2-{3-[(2-phenylethyl)amino]propyl}-4,5-dihydro-2H-pyrazolo[3,4-f]isoquinoline-3-carboxylic acid trifluoroacetate, 2-{3-[(thien-2-ylmethyl)amino]propyl}-4,5-dihydro-2H-pyrazolo[3,4-f]isoquinoline-3-carboxylic acid trifluoroacetate, 2-benzyl-5,6,9,10-tetrahydropyrazino[1′,2′:1,5]pyrazolo[3,4-f]isoquinolin-7(8H)-one, 5,6,8,9,10,11-hexahydro-7H-[1,4]diazepino[1′,2′:1,5]pyrazolo[3,4-f]isoquinolin-7-one, 2-{3-[(4-chlorobenzyl)amino]propyl}-4,5-dihydro-2H-pyrazolo[3,4-(]isoquinoline-3-carboxylic acid trifluoroacetate, 2-{3-[(2-phenylpropyl)amino]propyl}-4,5-dihydro-2H-pyrazolo[3,4-f]isoquinoline-3-carboxylic acid trifluoroacetate, 7-oxo-5,6,7,8,9,10-hexahydropyrazino[1′,2′: 1,5]pyrazolo[3,4-f]isoquinoline-9-carboxamide trifluoroacetate, 2-(3-hydroxypropyl)-4,5-dihydro-2H-pyrazolo[3,4-f]isoquinoline-3-carboxylic acid, 2-(1,3-dihydro-2H-isoindol-2-yl)-5,6,9,10-tetrahydropyrazino[1′,2′:1,5]pyrazolo[3,4-f]isoquinolin-7(8H)-one trifluoroacetate, 2-{3-[(4-aminophenyl)amino]propyl}-4,5-dihydro-2H-pyrazolo[3,4-(]isoquinoline-3-carboxylic acid trifluoroacetate, 2-(4-hydroxypiperidin-1-yl)-5,6,9,10-tetrahydropyrazino[1′,2′: 1,5]pyrazolo[3,4-f]isoquinolin-7(8H)-one trifluoroacetate, 2-(3-aminopropyl)-7-hydroxy-8-(3-nitrophenyl)-4,5-dihydro-2H-benzo[g]indazole-3-carboxylic acid trifluoroacetate, 2-(2-aminoethyl)-7-hydroxy-8-(3-nitrophenyl)-4,5-dihydro-2H-benzo[g]indazole-3-carboxylic acid trifluoroacetate, 3-hydroxy-2-(3-nitrophenyl)-5,6,8,9,10,11-hexahydro-7H-benzo[g][1,4]diazepino[1,2-b]indazol-7-one trifluoroacetate, 2-(3-aminopropyl)-7-hydroxy-4,5-dihydro-2H-benzo[g]indazole-3-carboxylic acid dihydrochloride, 2-(2-aminoethyl)-8-bromo-7-hydroxy-4,5-dihydro-2H-benzo[g]indazole-3-carboxylic acid trifluoroacetate, 2-(3-aminopropyl)-8-[2-(4-chlorophenyl)ethyl]-7-hydroxy-4,5-dihydro-2H-benzo[g]indazole-3-carboxylic acid trifluoroacetate, 3-hydroxy-2-(3-nitrophenyl)-5,6,9,10-tetrahydrobenzo[g]pyrazino[1,2-b]indazol-7(8H)-one hydrobromide, 2-(3-aminopropyl)-8-bromo-7-hydroxy-4,5-dihydro-2H-benzo[g]indazole-3-carboxylic acid hydrobromide, 2-(3-aminopropyl)-7-hydroxy-8-(4-nitrophenyl)-4,5-dihydro-2H-benzo[g]indazole-3-carboxylic acid trifluoroacetate, 2-(3-aminopropyl)-8-(3-cyanophenyl)-7-hydroxy-4,5-dihydro-2H-benzo[g]indazole-3-carboxylic acid trifluoroacetate, 2-(3-aminopropyl)-7-hydroxy-8-[3-(trifluoromethyl)phenyl]-4,5-dihydro-2H-benzo[g]indazole-3-carboxylic acid trifluoroacetate, and 2-(3-aminopropyl)-7-hydroxy-8-(3,3,3-trifluoropropyl)-4,5-dihydro-2H-benzo[g]indazole-3-carboxylic acid trifluoroacetate, 7-hydroxy-3,4,5,10-tetrahydro-1H-2,5-methanoazepino[3,4-b]indole-1-carboxylic acid, 2,3,8,10,11,12-hexahydro-1H,7H-9,12-methanoazepino[3,4-b]pyrano[3,2-e]indole-8-carboxylic acid, 7-methoxy-3,4,5,10-tetrahydro-1H-2,5-methanoazepino[3,4-b]indole-1-carboxylic acid, 7-(methylthio)-3,4,5,10-tetrahydro-1H-2,5-methanoazepino[3,4-b]indole-1-carboxylic acid, 7-(benzyloxy)-3,4,5,10-tetrahydro-1H-2,5-methanoazepino[3,4-b]indole-1-carboxylic acid, 7-(methylthio)-3,4,5,10-tetrahydro-1H-2,5-methanoazepino[3,4-b]indole-1-carboxylic acid, 2,2,2-trifluoroethyl 7-methoxy-3,4,5,10-tetrahydro-1H-2,5-methanoazepino[3,4-b]indole-1-carboxylate, 2,3-dihydroxypropyl 7-methoxy-3,4,5,10-tetrahydro-1H-2,5-methanoazepino[3,4-b]indole-1-carboxylate, pyridin-4-ylmethyl 7-methoxy-3,4,5,10-tetrahydro-1H-2,5-methanoazepino[3,4-b]indole-1-carboxylate, 2-fluoroethyl 7-methoxy-3,4,5,10-tetrahydro-1H-2,5-methanoazepino[3,4-b]indole-1-carboxylate, allyl 7-methoxy-3,4,5,10-tetrahydro-1H-2,5-methanoazepino[3,4-b]indole-1-carboxylate, benzyl 7-methoxy-3,4,5,10-tetrahydro-1H-2,5-methanoazepino[3,4-b]indole-1-carboxylate, 2-(methylthio)ethyl 7-methoxy-3,4,5,10-tetrahydro-1H-2,5-methanoazepino[3,4-b]indole-1-carboxylate, 2-methoxyethyl 7-methoxy-3,4,5,10-tetrahydro-1H-2,5-methanoazepino[3,4-b]indole-1-carboxylatem, 7-methoxy-3,4,5,10-tetrahydro-1H-2,5-methanoazepino[3,4-b]indole-1-carboxylic acid, 7-hydroxy-3,4,5,10-tetrahydro-1H-2,5-methanoazepino[3,4-b]indole-1-carboxylic acid, 2,3,8,10,11,12-hexahydro-1H,7H-9,12-methanoazepino[3,4-b]pyrano[3,2-e]indole-8-carboxylic acid, 7-methoxy-3,4,5,10-tetrahydro-1H-2,5-methanoazepino[3,4-b]indole-1-carboxylic acid, 7-(methylthio)-3,4,5,10-tetrahydro-1H-2,5-methanoazepino[3,4-b]indole-1-carboxylic acid, 7-methoxy-3,4,5,10-tetrahydro-1H-2,5-methanoazepino[3,4-b]indole-1-carboxylic acid, 7-(benzyloxy)-3,4,5,10-tetrahydro-1H-2,5-methanoazepino[3,4-b]indole-1-carboxylic acid, 7-(methylthio)-3,4,5,10-tetrahydro-1H-2,5-methanoazepino[3,4-b]indole-1-carboxylic acid, 6-methoxy-2,3,4,9-tetrahydro-1H-beta-carboline-1-carboxylic acid, 6-(2-oxo-2-phenylethoxy)-2,3,4,9-tetrahydro-1H-beta-carboline-1-carboxylic acid, 6-methoxy-2-methyl-2,3,4,9-tetrahydro-1H-beta-carboline-1-carboxylic acid, 2,2,2-trifluoroethyl 7-methoxy-3,4,5,10-tetrahydro-1H-2,5-methanoazepino[3,4-b]indole-1-carboxylate, 6-methoxy-2,3,4,9-tetrahydro-1H-beta-carboline-1-carboxylic acid, 7-hydroxy-3,4,5,10-tetrahydro-1H-2,5-methanoazepino[3,4-b] indole-1-carboxylic acid, 6-hydroxy-2-methyl-2,3,4,9-tetrahydro-1H-beta-carboline-1-carboxylic acid, 7-methoxy-3,4,5,10-tetrahydro-1H-2,5-methanoazepino[3,4-b]indole-1-carboxylic acid, 6-methoxy-2-methyl-2,3,4,9-tetrahydro-1H-beta-carboline-1-carboxylic acid, 2,3-dihydroxypropyl 7-methoxy-3,4,5,10-tetrahydro-1H-2,5-methanoazepino[3,4-b]indole-1-carboxylate, 4-ethyl-6-methoxy-2,3,4,9-tetrahydro-1H-beta-carboline-1-carboxylic acid, 6-methoxy-4-methyl-2,3,4,9-tetrahydro-1H-beta-carboline-1-carboxylic acid, 8,9,10,11-tetrahydro-7H-pyrido[3′,4′:4,5]pyrrolo[2,3-f]isoquinolin-7-one trifluoroacetate, 3-(aminomethyl)-6-methoxy-2,3,4,9-tetrahydro-1H-beta-carbolin-1-one trifluoroacetate, 3-(aminomethyl)-6-methoxy-2,3,4,9-tetrahydro-1H-beta-carbolin-1-one hydrochloride, 7-methoxy-3,4,5,10-tetrahydroazepino[3,4-b]indol-1 (2H)-one, 6-methoxy-2,3,4,9-tetrahydro-1H-beta-carbolin-1-one, 6-methoxy-2,9-dihydro-1H-beta-carbolin-1-one, 6-hydroxy-2,3,4,9-tetrahydro-1H-beta-carbolin-1-one, 8,9,10,11-tetrahydro-7H-pyrido[3′,4′:4,5]pyrrolo-[2,3-f]isoquinolin-7-one, 3-(aminomethyl)-6-methoxy-2,3,4,9-tetrahydro-1H-beta-carbolin-1-one, 3-(aminomethyl)-6-methoxy-2,3,4,9-tetrahydro-1H-beta-carbolin-1-one, 6-methoxy-3-{3-[(2-phenylethyl)amino]propyl}-2,3,4,9-tetrahydro-1H-beta-carbolin-1-one, (1E)-6-methoxy-2,3,4,9-tetrahydro-1H-carbazol-1-one oxime, (1Z)-6-methoxy-2,3,4,9-tetrahydro-1H-carbazol-1-one oxime, 6-methoxy-3-{3-[(3-phenylpropyl)amino]propyl}-2,3,4,9-tetrahydro-1H-beta-carbolin-1-one, methyl 1-oxo-2,3,4,5-tetrahydro-1H-pyrido[4,3-b]indole-6-carboxylate, 3-(hydroxymethyl)-6-methoxy-2,3,4,9-tetrahydro-1H-beta-carbolin-1-one, 3-(3-aminopropyl)-6-methoxy-2,3,4,9-tetrahydro-1H-beta-carbolin-1-one, 3-(2-aminoethyl)-6-methoxy-2,3,4,9-tetrahydro-1H-beta-carbolin-1-one, ethyl 1-(hydroxyimino)-2,3,4,9-tetrahydro-1H-carbazole-6-carboxylate, 2-methoxy-7,8,9,10-tetrahydrocyclohepta[b]indol-6(5H)-one oxime, 3-(hydroxymethyl)-6-methoxy-2,3,4,9-tetrahydro-1H-beta-carbolin-1-one, 3-(3-aminopropyl)-6-methoxy-2,3,4,9-tetrahydro-1H-beta-carbolin-1-one, 3-(2-aminoethyl)-6-methoxy-2,3,4,9-tetrahydro-1H-beta-carbolin-1-one, ethyl 1-(hydroxyimino)-2,3,4,9-tetrahydro-1H-carbazole-6-carboxylate, 2-methoxy-7,8,9,10-tetrahydrocyclohepta[b]indol-6(5H)-one oxime, 3-[3-(benzylamino)propyl]-6-methoxy-2,3,4,9-tetrahydro-1H-beta-carbolin-1-one, 6-methoxy-2,3,4,9-tetrahydro-1H-carbazol-1-one oxime, 6-iodo-2,3,4,9-tetrahydro-1H-carbazol-1-one oxime, 6-methoxy-2-methyl-2,3,4,9-tetrahydro-1H-carbazol-1-one oxime, 3-(3-hydroxypropyl)-6-methoxy-2,3,4,9-tetrahydro-1H-beta-carbolin-1-one, ethyl 1-oxo-2,3,4,5-tetrahydro-1H-pyrido[4,3-b]indole-6-carboxylate, 6-methoxy-2,3,4,9-tetrahydro-1H-beta-carboline-1-thione, methyl 4-oxo-2,3,4,9-tetrahydro-1H-carbazole-8-carboxylate, and 2,3,4,9-tetrahydro-1H-carbazol-1-one oximc.

In additional examples of the above methods, the MK2 inhibitor may be an siRNA molecule containing the sequence of any one of CGAUGCGUGUUGACUAUGAdTdT (SEQ ID NO: 1), UCAUAGUCAACACGCAUCGdTdT (SEQ ID NO: 2), UGACCAUC ACCGAGUUUAU dTdT (SEQ ID NO: 3), and AUAAACUCGGUGAUGGUCAdTdT (SEQ ID NO: 4). In further examples of the above methods, the MK2 inhibitor may be a nucleobase oligomer containing a sequence complementary to at least 10 consecutive nucleotides of a nucleic acid sequence encoding a MK2 protein. In additional examples of the above methods, the MK2 inhibitor is a peptide containing the amino acid sequence of [L/F/I]XR[Q/S/T]L[S/T][hydrophobic] (SEQ ID NO: 5), where the peptide contains no more than 50 amino acids (e.g., a peptide containing the amino acid sequence of LQRQLSI (SEQ ID NO: 6)). In further examples of the above methods, the MK2 inhibitor is a peptide that contains a covalently-linked moiety capable of tranlocating across a biological membrane (e.g., a moiety that contains a penetratin peptide or a TAT peptide).

The invention further provides methods of reducing the severity of one or more symptoms of cancer in a patient requiring the steps of: (i) measuring one or more feature(s) (e.g., at least two) in a cancer cell(s) from the patient selected from the group of cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phorphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; (ii) determining from the measurements in step (i) whether the cancer cell(s) in the patient has one or more feature(s) of an inactivated MK2 signaling pathway selected from the group of: decreased cytoplasmic MK2 protein localization, increased nuclear MK2 protein localization, decreased phosphorylation of total MK2 protein, decreased levels of phosphorylated MK2 protein in the cytoplasm or nucleus, decreased levels of phosphorylated hsp27, decreased levels of phosphorylated hnRNPA0, decreased levels of phosphorylated PARN, decreased levels of phosphorylated TIAR, decreased levels of phosphorylated cdc25B, decreased levels of phosphorylated cdc25C, and decreased levels of Gadd45a protein or mRNA relative to these features in a control sample; and (iii) administering to a patient determined to have a cancer cell having one or more the feature(s) of an inactivated MK2 signaling pathway one or more chemotherapeutic agent(s) (e.g., a chemotherapeutic agent that induces DNA damage) for a time and in an amount sufficient to reduce the severity of one or more symptom(s) of cancer in the patient.

Additional embodiments of the above methods further require the steps of: (iv) measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; (v) determining from the measurements in step (iv) whether the cancer cell(s) in the patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; and (vi) administering to a patient determined to have a cancer cell having one or more the feature(s) of an inactivated MK2 signaling pathway and one or more the feature(s) of an inactivated p53 pathway one or more chemotherapeutic agent(s) (e.g., a chemotherapeutic agent that induces DNA damage) for a time and in an amount sufficient to reduce the severity of one or more symptom(s) of cancer in the patient. In additional embodiments of the above methods, the control sample in step (ii) is a cancer cell or non-cancerous cell treated with a genotoxic agent and/or the control sample in step (v) is a non-cancerous cell.

The invention also provides methods of identifying a cancer patient that may selectively benefit from the administration of one or more MK2 inhibitor(s) or the administration of the combination of one or more MK2 inhibitor(s) and one or more chemotherapeutic agent(s) requiring the steps of: (i) measuring one or more feature(s) (e.g., at least two) in a cancer cell(s) from the patient selected from the group of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phorphorylated poly(A)-specific ribonuclease (PARN), and levels of growth arrest and DNA-damage-inducible-45A (Gadd45a) protein or mRNA; and (ii) determining from the measurements in step (i) whether the cancer cell(s) in the patient has one or more feature(s) of an activated MK2 signaling pathway selected from the group of: increased cytoplasmic MK2 protein localization, decreased nuclear MK2 protein localization, increased phosphorylation of total MK2 protein, increased levels of phosphorylated MK2 protein in the cytoplasm or nucleus, increased levels of phosphorylated hsp27, increased levels of phosphorylated hnRNPA0, increased levels of phosphorylated PARN, increased levels of phosphorylated TIAR, increased levels of phosphorylated cdc25B, increased levels of phosphorylated cdc25C, and increased levels of Gadd45a protein or mRNA relative to these features in a control sample; where a patient having one or more the feature(s) of an activated MK2 signaling pathway is identified as a cancer patient that may selectively benefit from the administration of one or more MK2 inhibitor(s) or the administration of the combination of one or more MK2 inhibitor(s) and one or more chemotherapeutic agent(s).

Additional embodiments of the above methods further require the steps of: (iii) measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; and (iv) determining from the measurements in step (iii) whether the cancer cell(s) in the patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; where a patient having one or more the feature(s) of an activated MK2 signaling pathway and one or more the feature(s) of an inactivated p53 pathway is identified as a cancer patient that may selectively benefit from the administration of one or more MK2 inhibitor(s) or the administration of the combination of one or more MK2 inhibitor(s) and one or more chemotherapeutic agent(s).

In any of the above methods, the control sample in step (ii) is a non-cancerous cell or a cell untreated with a genotoxic agent and/or the control sample in step (iv) is a non-cancerous cell. In any of the above methods, the cancer patient may have previously received a dosage of a chemotherapeutic agent.

The invention also provides methods of identifying a cancer patient that may selectively benefit from the administration of dosage of one or more chemotherapeutic agent(s) requiring the steps of: (i) measuring one or more (e.g., at least two) the feature(s) in a cancer cell(s) from the patient selected from the group of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and (ii) determining from the measurements in step (i) whether the cancer cell(s) in the patient has one or more feature(s) of an inactivated MK2 signaling pathway selected from the group of: decreased cytoplasmic MK2 protein localization, increased nuclear MK2 protein localization, decreased phosphorylation of total MK2 protein, decreased levels of phosphorylated MK2 protein in the cytoplasm or nucleus, decreased levels of phosphorylated hsp27, decreased levels of phosphorylated hnRNPA0, decreased levels of phosphorylated PARN, decreased levels of phosphorylated TIAR, decreased levels of phosphorylated cdc25B, decreased levels of phosphorylated cdc25C, and decreased levels of Gadd45a protein or mRNA relative to these features in a control sample; where a patient having one or more the feature(s) of an inactivated MK2 signaling pathway is identified as a cancer patient that may selectively benefit from the administration of one or more chemotherapeutic agent(s).

Additional embodiments of the above methods further require the steps of: (iii) measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; and (iv) determining from the measurements in step (iii) whether the cancer cell(s) in the patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; where a patient having one or more the feature(s) of an inactivated MK2 signaling pathway and one or more the feature(s) of an inactivated p53 pathway is identified as a cancer patient that may selectively benefit from the administration of one or more chemotherapeutic agent(s).

In any of the above methods, the patient may have previously received at least one dosage of a chemotherapeutic agent. In additional embodiments of the above methods, the control sample in step (ii) is a cancer cell or non-cancerous cell treated with a genotoxic agent and/or the control sample in step (iv) is a non-cancerous cell.

The invention further provides methods of diagnosing a chemotherapy-resistant cancer in a patient requiring the steps of: (i) measuring one or more (e.g., at least two) feature(s) in a cancer cell(s) from the patient selected from the group of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat-shock protein-27 (hsp27), levels of phosphorylated hnRNPA0, levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and (ii) determining from the measurements in step (i) whether the cancer cell(s) in the patient has one or more feature(s) of an activated MK2 signaling pathway selected from the group of: increased cytoplasmic MK2 protein localization, decreased nuclear MK2 protein localization, increased phosphorylation of total MK2 protein, increased levels of phosphorylated MK2 protein in the cytoplasm or nucleus, increased levels of phosphorylated hsp27, increased levels of phosphorylated hnRNPA0, increased levels of phosphorylated PARN, increased levels of phosphorylated TIAR, increased levels of phosphorylated cdc25B, increased levels of phosphorylated cdc25C, and increased levels of Gadd45a protein or mRNA relative to these features in a control sample; where a cancer cell having one or more the feature(s) of an activated MK2 signaling pathway indicates that the patient has a chemotherapy-resistant cancer.

Additional embodiments of the above methods further require the steps of: (iii) measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; and (iv) determining from the measurements in step (iii) whether the cancer cell(s) in the patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; where a cancer cell having one or more the feature(s) of an activated MK2 signaling pathway and one or more the feature(s) of an inactivated p53 signaling pathway indicates that the patient has a chemotherapy-resistant cancer.

In additional examples of the above methods, the control sample in step (ii) is a non-cancerous cell or a cell untreated with a genotoxic agent and/or the control sample in step (iv) is a non-cancerous cell.

The invention further provides methods of diagnosing a chemotherapy-sensitive cancer in a patient requiring the steps of: (i) measuring one or more (e.g., at least two) feature(s) in a cancer cell(s) from the patient selected from the group of cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated hnRNPA0, levels of phorphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and (ii) determining from the measurements in step (i) whether the cancer cell(s) in the patient has one or more feature(s) of an inactivated MK2 signaling pathway selected from the group of: decreased cytoplasmic MK2 protein localization, increased nuclear MK2 protein localization, decreased phosphorylation of total MK2 protein, decreased levels of phosphorylated MK2 protein in the cytoplasm or nucleus, decreased levels of phosphorylated hsp27, decreased levels of phosphorylated hnRNPA0, decreased levels of phosphorylated PARN, decreased levels of phosphorylated TIAR, decreased levels of phosphorylated cdc25B, decreased levels of phosphorylated cdc25C, and decreased levels of Gadd45a protein or mRNA relative to these features in a control sample; where a cancer cell having one or more the feature(s) of an inactivated MK2 signaling pathway indicates that the patient has a chemotherapy-sensitive cancer.

Additional embodiments of these methods require the steps of: (iii) measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; and (iv) determining from the measurements in step (iii) whether the cancer cell(s) in the patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; where a cancer cell having one or more the feature(s) of an inactivated MK2 signaling pathway and one or more the feature(s) of an inactivated p53 signaling pathway indicates that the patient has a chemotherapy-sensitive cancer.

In any of the above methods, the control sample in step (ii) is a cancer cell or non-cancerous cell treated with a genotoxic agent and/or the control sample in step (iv) is a non-cancerous cell.

The invention further provides methods of treating a cancer patient diagnosed as having a chemotherapy-resistant cancer by any of the above methods, requiring the step of administering to the patient one or more MK2 inhibitor(s). These methods may further include administering one or more chemotherapeutic agent(s) (e.g., a chemotherapeutic agent that induces DNA damage) to the patient. Any of the above described MK2 inhibitors and/or chemotherapeutic agents may be used in these methods.

The invention further provides methods of treating a cancer patient diagnosed as having a chemotherapy-sensitive cancer by any of the above methods, comprising the step of administering to the patient one or more chemotherapeutic agent(s) (e.g., a chemotherapeutic agent that induces DNA damage). Any of the above described chemotherapeutic agents may be used in these methods.

In any of the above methods, the chemotherapeutic agent may be selected from the group of: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine.

In additional embodiments of any of the above methods, the cancer may be selected from the group of acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor. In additional examples of any of the above methods, the cancer cell(s) are from a biopsy sample from the patient.

The invention further provides kits for diagnosing a chemotherapy-resistant or chemotherapy-sensitive cancer in a patient containing: (a) one or more reagent(s) capable of measuring one or more feature(s) in a cancer cell(s) from the patient selected from the group of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated hnRNPA0, levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and (b) instructions for using the reagents of (a) to determine the presence of a chemotherapy-resistant or chemotherapy-sensitive cancer in the patient.

Additional embodiments of the above kits may further include: (c) one or more reagent(s) capable of measuring one or more feature(s) in a cancer cell(s) from the patient selected from the group of: tumor protein-53 (p53) mRNA or protein levels, expression of a mutant or truncated p53 with decreased expression or activity, and cyclin-dependent kinase inhibitor 1 (p21) expression or activity; and (d) instructions for using the reagents of (a) and (b) to determine the presence of a chemotherapy-resistant or chemotherapy-sensitive cancer in the patient.

In additional embodiments of the provided kits, the one or more reagent(s) in (a) are selected from the group of: an antibody that binds phosphorylated, nonphosphorylated, or total MK2 protein; an antibody that binds phosphorylated, nonphosphorylated, or total hsp27; an antibody that binds to phosphorylated, nonphosphorylated, or total hnRNPA0; an antibody that binds to phosphorylated, nonphosphorylated, or total PARN; an antibody that binds to phosphorylated, nonphosphorylated, or total TIAR; an antibody that binds to Gadd45a; an antibody that binds to phosphorylated, nonphosphorylated, or total cdc25B; an antibody that binds to phosphorylated, nonphosphorylated, or total cdc25C; an oligonucleotide containing a sequence complementary to a nucleic acid sequence encoding Gadd45a protein; and one or more nucleic acid primer(s) complementary to a sequence in Gadd45a mRNA. In additional examples of the above kits, the one or more reagent(s) in (b) are selected from the group of: an antibody binding to p53 protein; an oligonucleotide containing a sequence complementary to a nucleic acid sequence encoding a wild type p53 protein; one or more nucleic acid primer(s) complementary to a nucleic acid sequence encoding a wild type p53 protein; an oligonucleotide containing a sequence complementary to a nucleic acid sequence encoding a mutant or truncated p53 protein; one or more nucleic acid primer(s) complementary to a nucleic acid sequence encoding a mutant or truncated p53 protein; and an antibody that binds to p21. In additional embodiments of any of the above kits, the nucleic acid sequence encoding a wild type, mutant, or truncated p53 is an mRNA or a genomic DNA sequence.

As used throughout this specification and the appended claims, the following terms have the meanings specified.

By “antisense,” as used herein in reference to nucleic acids, is meant a nucleic acid sequence, regardless of length, that is complementary to the coding strand of a gene.

By “binding to” a molecule is meant having a physicochemical affinity for that molecule. For example, an antibody molecule may have affinity for an epitope found in a target protein.

By “cancer” is meant a disease characterized by the pathological proliferation of a cell or tissue and its subsequent migration to or invasion of other tissues or organs. Cancer growth is typically uncontrolled and progressive, and occurs under conditions that would not elicit, or would cause cessation of, multiplication of normal cells. Cancers can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Non-limiting examples of cancers include: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

By “cell division cycle 25B” or “cdc25B” is meant is meant a protein substantially identical to NCBI Accession No. AAH09953.1 (SEQ ID NO: 31) or AAR26469.1 (SEQ ID NO: 32), or a nucleic acid encoding a protein substantially identical to NCBI Accession No. AAH09953.1 (SEQ ID NO: 31) or AAR26469.1 (SEQ ID NO: 32).

By “phosphorylated cdc25B” is meant a cdc25B protein that has been phosphorylated. For example, the term phosphorylated cdc25B includes a cdc25B protein that is phosphorylated at serine-323.

By “cell division cycle 25C” or “cdc25C” is meant is meant a protein substantially identical to any one of NCBI Accession Nos. EAW62150.1 (SEQ ID NO: 33), EAW62149.1 (SEQ ID NO: 34), EAW62148.1 (SEQ ID NO: 35), EAW62147.1 (SEQ ID NO: 36), EAW62146.1 (SEQ ID NO: 37), EAW62145.1 (SEQ ID NO: 38), and AAR32098.1 (SEQ ID NO: 39), or a nucleic acid encoding a protein substantially identical to any one of NCBI Accession Nos. EAW62150.1 (SEQ ID NO: 33), EAW62149.1 (SEQ ID NO: 34), EAW62148.1 (SEQ ID NO: 35), EAW62147.1 (SEQ ID NO: 36), EAW62146.1 (SEQ ID NO: 37), EAW62145.1 (SEQ ID NO: 38), and AAR32098.1 (SEQ ID NO: 39).

By “phosphorylated cdc25C” is meant a cdc25C protein that has been phosphorylated. For example, the term phosphorylated cdc25C includes a cdc25C protein that is phosphorylated at serine-216.

By “chemotherapeutic agent” is meant one or more chemical agents used in the treatment or control of proliferative diseases (e.g., cancer). Chemotherapeutic agents include cytotoxic and cytostatic agents. Exemplary chemotherapeutic agents may mediate DNA damage (e.g., alkylating chemotherapeutic agents). Non-limiting examples of chemotherapeutic agents are described herein and are known in the art.

By “control sample” is meant a cell, cell sample, or protein or DNA sample that is used as a reference. For example, in experiments to determine activation of the MK2 signaling pathway, the control sample may be a non-cancer cell (e.g., a non-cancer cell from a patient) or a cell that is not treated a genotoxic agent (e.g., a DNA-damaging chemotherapeutic agent), or a lysate prepared from such a cell. In experiments to determine inactivation of the MK2 signaling pathway, the control sample may be a cell that has been treated with a genotoxic agent (e.g., a DNA-damaging chemotherapeutic agent).

By “detectably-labeled” is meant any means for marking and identifying the presence of a target molecule in a cell or a cell lysate. For example, antibodies or antisense nucleic acid molecules that recognize a target protein (e.g., MK2 or p53 protein), mRNA (e.g., a MK2 or p53 mRNA), or genomic DNA (e.g., gene encoding wild type, mutant, or truncated p53) in a cell or cell lysate may be detectably-labeled. Methods for detectably-labeling a molecule are well known in the art and include, without limitation, radionuclides (e.g., with an isotope such as ³²P, ³³P, ¹²⁵I, or ³⁵S), nonradioactive labeling (e.g., chemiluminescent labeling or fluorescein labeling), and epitope tags.

By “genotoxic agent” is meant any agent that causes, directly or indirectly, DNA damage in a cell. Non-limiting examples of genotoxic agents include DNA-damaging chemotherapeutic agents (e.g., doxorubicin), intercalating agents, UV light, and alkylating agents. Additional examples of genotoxic agents are known in the art.

By “growth arrest and DNA-damage-inducible-45A” or “Gadd45A” is meant a protein substantially identical to any one of NCBI Accession Nos. CAI23495.1 (SEQ ID NO: 40), NP_{—}001915.1 (SEQ ID NO: 41), EAX06488.1 (SEQ ID NO: 42), EAX06487.1 (SEQ ID NO: 43), AAM88884.1 (SEQ ID NO: 44), AAH11757.1 (SEQ ID NO: 45), CAI23494.1 (SEQ ID NO: 46), ABQ52427.1 (SEQ ID NO: 47), AAY25021.1 (SEQ ID NO: 48), P24522.1 (SEQ ID NO: 49), and NP_{—}056490.2 (SEQ ID NO: 50), or a nucleic acid encoding a protein substantially identical to any one of NCBI Accession Nos. CAI23495.1 (SEQ ID NO: 40), NP_{—}001915.1 (SEQ ID NO: 41), EAX06488.1 (SEQ ID NO: 42), EAX06487.1 (SEQ ID NO: 43), AAM88884.1 (SEQ ID NO: 44), AAH11757.1 (SEQ ID NO: 45), CAI23494.1 (SEQ ID NO: 46), ABQ52427.1 (SEQ ID NO: 47), AAY25021.1 (SEQ ID NO: 48), P24522.1 (SEQ ID NO: 49), and NP_{—}056490.2 (SEQ ID NO: 50).

By “heat shock protein-27” or “hsp27” is meant a protein substantially identical to any one of NCBI Accession Nos. BAB17232 (SEQ ID NO: 51), AAH12292.1 (SEQ ID NO: 52), AAH73768.1 (SEQ ID NO: 53), AAH12768.1 (SEQ ID NO: 54), AAH00510.1 (SEQ ID NO: 55), and P04792.2 (SEQ ID NO: 56) or a nucleic acid encoding a protein substantially identical to any one of NCBI Accession Nos. BAB17232 (SEQ ID NO: 51), AAH12292.1 (SEQ ID NO: 52), AAH73768.1 (SEQ ID NO: 53), AAH12768.1 (SEQ ID NO: 54), AAH00510.1 (SEQ ID NO: 55), and P04792.2 (SEQ ID NO: 56).

By “phosphorylated hsp27” is meant a hsp27 protein that has been phosphorylated. For example, the term phosphorylated hsp27 includes an hsp27 protein that is phosphorylated at serine 15, serine 78, and/or serine 82.

By “hnRNPA0” or “heterogeneous nuclear ribonucleoprotein A0” is meant a protein substantially identical to any one of NCBI Accession Nos. NP_{—}006796 (SEQ ID NO: 57), AAH30249.1 (SEQ ID NO: 58), AAH28976.1 (SEQ ID NO: 59), AAH19271.1 (SEQ ID NO: 60), AAH11972.1 (SEQ ID NO: 61), AAH07271.1 (SEQ ID NO: 62), AAH01008.1 (SEQ ID NO: 63), AAH18949.1 (SEQ ID NO: 64), AAH12980.1 (SEQ ID NO: 65), AAH09284.1 (SEQ ID NO: 66), and Q13151 (SEQ ID NO: 67), or a nucleic acid encoding a protein substantially identical to any one of NCBI Accession Nos. NP_{—}006796 (SEQ ID NO: 57), AAH30249.1 (SEQ ID NO: 58), AAH28976.1 (SEQ ID NO: 59), AAH19271.1 (SEQ ID NO: 60), AAH11972.1 (SEQ ID NO: 61), AAH07271.1 (SEQ ID NO: 62), AAH01008.1 (SEQ ID NO: 63), AAH18949.1 (SEQ ID NO: 64), AAH12980.1 (SEQ ID NO: 65), AAH09284.1 (SEQ ID NO: 66), and Q13151 (SEQ ID NO: 67).

By “phosphorylated hnRNPA0” is meant an hnRNPA0 protein that has been phosphorylated. For example, phosphorylated hnRNPA0 includes an hnRNPA0 protein that is phosphorylated at serine-84.

By “hydrophobic” in the context of amino acids is meant any of the following amino acids: alanine, cysteine, isoleucine, leucine, methionine, phenylalanine, proline, tryptophan, tyrosine, or valine.

By “MK2 biological activity” is meant any activity known to be caused in vivo or in vitro by a MK2 polypeptide. For example, such activity could be caused by at least one of the following: function in a DNA damage response pathway, cell cycle control, transcriptional regulation, chromatin remodeling, nuclear export (e.g., translocation from the nucleus to the cytoplasm), or substrate binding. In one assay for MK2 biological activity, the ability of MK2, or a fragment or mutant thereof containing a substrate-binding domain, to bind a substrate is measured. In another assay for MK2 biological activity, the ability of MK2 to phosphorylate a substrate (e.g., hsp27, hnRNPA0, PARN, cdc25B, and cdc25C) is measured.

By “MK2 inhibitor” is meant a compound that is able to reduce (e.g., by at least 5%, 10%, 15%, 20%, 25%, 30%, 35%, 40%, 45%, 50%, 55%, 60%, 65%, 70%, 75%, 80%, 85%, 90%, or 95%) the expression (e.g., protein or mRNA) or reduce (e.g., by at least 5%, 10%, 15%, 20%, 25%, 30%, 35%, 40%, 45%, 50%, 55%, 60%, 65%, 70%, 75%, 80%, 85%, 90%, or 95%) one or more biological activities of MK2 polypeptide. Examples of MK2 inhibitors include small molecules (e.g., UCN-01), peptides, siRNA molecules, antisense nucleic acids, and antibodies. Non-limiting examples of MK2 inhibitors are described herein and in U.S. Patent Application Publication Nos. 2009/0010927, 2006/0115453, and 2009/0181468, and International Patent Application Publication Nos. WO 05/115454 and WO 06/053315, each of which is incorporated herein in their entirety.

By “MK2 nucleic acid” is meant a nucleic acid that encodes all or a portion of a MK2 polypeptide or is substantially identical (e.g., at least 80%, 85%, 90%, 95%, 96%, 97%, 98%, 99%, or 100%) to all or a portion of the nucleic acid sequence of Genbank Accession Nos. NM_{—}004759 (SEQ ID NO: 22) or NM_{—}032960 (SEQ ID NO: 23), or analog thereof.

By “MK2 polypeptide” is meant a polypeptide substantially identical (e.g., at least 80%, 85%, 90%, 95%, 96%, 97%, 98%, 99%, or 100%) to all or a portion of the polypeptide sequence of Genbank Accession Nos. NP_{—}004750 (SEQ ID NO: 24) or P49137 (SEQ ID NO: 25), or analog thereof, and having one or more (e.g., two, three, or four) MK2 biological activity.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred nucleic acids may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines.

By “p53 levels” or “p53 expression” is meant the amount of p53 protein or p53 mRNA present in a cell (e.g., a cancer cell or a control cell).

By “p53 protein” is meant a protein that is substantially identical to all or a part of any one of NCBI Accession Nos. BAC16799.1 (SEQ ID NO: 68), AAC12971.1 (SEQ ID NO: 69), P04637.4 (SEQ ID NO: 70), NP_{—}000537.3 (SEQ ID NO: 71), NP_{—}001119584.1 (SEQ ID NO: 72), AAD28535.1 (SEQ ID NO: 73), and AAD28628.1 (SEQ ID NO: 74).

By “p53 mRNA” is meant an mRNA that encodes a protein that is substantially identical to all or a part of any one of NCBI Accession Nos. BAC16799.1 (SEQ ID NO: 68), AAC12971.1 (SEQ ID NO: 69), P04637.4 (SEQ ID NO: 70), NP_{—}000537.3 (SEQ ID NO: 71), NP_{—}001119584.1 (SEQ ID NO: 72), AAD28535.1 (SEQ ID NO: 73), and AAD28628.1 (SEQ ID NO: 74).

By “p53 gene” or “p53 genomic DNA” is meant a sequence of genomic DNA that encodes a wild type, mutant, or truncated p53 protein that encodes a protein that is substantially identical to all or a part of (e.g., at least 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, or 390 amino acids) any one of NCBI Accession Nos. BAC16799.1 (SEQ ID NO: 68), AAC12971.1 (SEQ ID NO: 69), P04637.4 (SEQ ID NO: 70), NP_{—}000537.3 (SEQ ID NO: 71), NP_{—}001119584.1 (SEQ ID NO: 72), AAD28535.1 (SEQ ID NO: 73), and AAD28628.1 (SEQ ID NO: 74). For example, a mutant p53 gene may encode a p53 protein that contains at one or more (e.g., at least two, three, four, five, six, seven, eight, nine, or ten) amino acid substitutions, deletions, and/or additions. A mutant p53 gene may encode a p53 protein that contains at least a 5 amino acid truncation (e.g., at least a 10, 15, 20, 25, 30, 35, 40, 45, 50, 60, 70, 80, 90, 100, 110, 120, 130, 140, 150, 160, 170, 180, 190, or 200 amino acid truncation) or at least a 5 amino acid addition (e.g., at least a 10, 15, 20, 25, 30, 35, 40, 45, 50, 60, 70, 80, 90, 100, 110, 120, 130, 140, 150, 160, 170, 180, 190, or 200 amino acid addition) (e.g., a fusion protein resulting from a gene translocation).

By “mutant or truncated p53 with reduced expression or activity” is meant a p53 protein that contains at least one amino acid substitution, deletion, and/or addition compared to the wild type sequence of p53 protein (or an mRNA encoding such a p53 protein) that results in a decrease in expression of p53 protein or a decrease in p53 activity in the cell. For example, a mutant p53 protein may contain one or more (e.g., at least two, three, four, five, six, seven, eight, nine, or ten) amino acid substitutions, deletions, and/or additions (e.g., a fusion protein resulting from a gene translocation) that decreases the ability of p53 to bind to DNA, mediate cell cycle arrest in response to genotoxic stress, and/or stimulate p21 gene expression. A mutant p53 protein may contain at least a 5 amino acid truncation (e.g., at least a 10, 15, 20, 25, 30, 35, 40, 45, 50, 60, 70, 80, 90, 100, 110, 120, 130, 140, 150, 160, 170, 180, 190, or 200 amino acid truncation) or at least a 5 amino acid addition (e.g., at least a 10, 15, 20, 25, 30, 35, 40, 45, 50, 60, 70, 80, 90, 100, 110, 120, 130, 140, 150, 160, 170, 180, 190, or 200 amino acid addition) (e.g., a fusion protein resulting from a gene translocation) compared to the wild type p53 protein. Several examples of mutant or truncated p53 are known in the art. In addition, a mutant p53 protein may result from a mutation in one or both alleles of a p53 gene. For example, a mutation in the second allele of a p53 gene may be detected in a cell having a mutation in the first allele of a p53 gene (e.g., a loss of heterozygosity mutation).

By “p53 activity” is meant an activity of wild type p53 protein in a cell. Non-limiting examples of p53 activity include DNA-binding activity, ability to mediate cell cycle arrest, and induction of p21 gene expression. Assays for measuring in vitro and in vivo p53 activity are known in the art.

By “p21 mRNA” is meant an mRNA that encodes a protein that is substantially identical to all or a portion of the polypeptide sequence of Genbank Accession Nos. AAB29246.1 (SEQ ID NO: 75), P38936.3 (SEQ ID NO: 76), AAH01935.1 (SEQ ID NO: 77), AAH00275.1 (SEQ ID NO: 78), AAH13967.1 (SEQ ID NO: 79), AAH00312.1 (SEQ ID NO: 80), NP_{—}510867.1 (SEQ ID NO: 81), and NP_{—}000380.1 (SEQ ID NO: 82).

By “p21 polypeptide” is meant a polypeptide substantially identical to all or a portion of the polypeptide sequence of Genbank Accession Nos. AAB29246.1 (SEQ ID NO: 75), P38936.3 (SEQ ID NO: 76), AAH01935.1 (SEQ ID NO: 77), AAH00275.1 (SEQ ID NO: 78), AAH13967.1 (SEQ ID NO: 79), AAH00312.1 (SEQ ID NO: 80), NP 510867.1 (SEQ ID NO: 81), and NP_{—}000380.1 (SEQ ID NO: 82).

By “p21 expression” is meant the level of p21 protein or p21 mRNA in a cell.

By “p21 activity” is meant at least one activity of wild type p21 protein. Non-limiting examples of p21 activity include cyclin-dependent kinase (CDK) inhibition (e.g., inhibition of the kinase activity of cyclin E/CDK2 and/or cyclin D/CDK4 complexes), the ability to bind CDK proteins, and the ability to mediate cell cycle arrest following genoxotic stress. A decrease in p21 activity is meant at least a 5% decrease (e.g., at least 10%, 15%, 20%, 25%, 30%, 35%, 40%, 45%, 50%, 55%, 60%, 65%, 70%, 75%, 80%, 85%, 90%, or 95% decrease) in one or more of the above p21 activities.

By “pharmaceutically acceptable excipient” is meant a carrier that is physiologically acceptable to the subject to which it is administered and that preserves the therapeutic properties of the compound with which it is administered. One exemplary pharmaceutically acceptable excipient is physiological saline. Other physiologically acceptable excipients and their formulations are known to one skilled in the art and described, for example, in “Remington: The Science and Practice of Pharmacy,” (20th ed., ed. A. R. Gennaro, 2000, Lippincott Williams & Wilkins).

By “prodrug” is meant a compound that is modified in vivo, resulting in formation of a biologically active drug compound, for example by hydrolysis in blood. A thorough discussion of prodrug modifications is provided in T. Higuchi and V. Stella, Pro-drugs as Novel Delivery Systems, Vol. 14 of the A.C.S. Symposium Series, Edward B. Roche, Ed., Bioreversible Carriers in Drug Design, American Pharmaceutical Association and Pergamon Press, 1987, and Judkins et al., Synthetic Communications 26(23):4351-4367, 1996, each of which is incorporated herein by reference.

By “poly(A)-specific ribonuclease” or “PARN” is meant a protein substantially identical to any one of NCBI Accession Nos. NP_{—}002573.1 (SEQ ID NO: 83), AAH50029.1 (SEQ ID NO: 84), 095453.1 (SEQ ID NO: 85), and CAA06683.1 (SEQ ID NO: 86), or a nucleic acid encoding a protein substantially identical to any one of NCBI Accession Nos. NP_{—}002573.1 (SEQ ID NO: 83), AAH50029.1 (SEQ ID NO: 84), O95453.1 (SEQ ID NO: 85), and CAA06683.1 (SEQ ID NO: 86).

By “phosphorylated PARN” is meant a PARN protein that has been phosphorylated. For example, the term phosphorylated PARN includes a PARN protein that is phosphorylated at serine-557.

By “reducing the severity of one or more symptoms” is meant a reduction (e.g., at least 5%, 10%, 15%, 20%, 25%, 30%, 35%, 40%, 45%, 50%, 55%, 60%, 65%, 70%, 75%, 80%, 85%, 90%, 95%, or 100%) in the severity or duration of at least one (e.g., at least two, three, four, five, or six) symptoms of a disease (e.g., a cancer). For example, the methods of the invention may result in at a 10% reduction in at least one (e.g., at least two, three, four, five, or six) symptoms of cancer.

By “RNA interference” (RNAi) is meant a phenomenon where double-stranded RNA homologous to a target mRNA leads to degradation of the targeted mRNA (e.g., a MK2 mRNA). RNAi is more broadly defined as degradation of target mRNAs by homologous siRNAs.

By “siNA” is meant small interfering nucleic acids. One exemplary siNA is composed of ribonucleic acid (siRNA). siRNAs can be 21-25 nt RNAs derived from processing of linear double-stranded RNA. siRNAs assemble in complexes termed RISC (RNA-induced silencing complex) and target homologous RNA sequences for endonucleolytic cleavage. Synthetic siRNAs also recruit RISCs and are capable of cleaving homologous RNA sequences.

By “substantially identical” is meant a polypeptide or nucleic acid exhibiting at least 75%, 80%, 85%, 90%, 91%, 92%, 93%, 94, 95%, 96%, 97%, 98%, 99%, or even 100% identity to a reference amino acid or nucleic acid sequence. For polypeptides, the length of comparison sequences will generally be at least 35 amino acids, 45 amino acids, 55 amino acids, or even 70 amino acids. For nucleic acids, the length of comparison sequences will generally be at least 60 nucleotides, 90 nucleotides, or even 120 nucleotides.

Sequence identity is typically measured using publicly available computer programs. Computer program methods to determine identity between two sequences include, but are not limited to, the GCG program package (Devereux et al., Nucleic Acids Research 12: 387, 1984), BLASTP, BLASTN, and FASTA (Altschul et al., J. Mol. Biol. 215:403, 1990). The well-known Smith Waterman algorithm may also be used to determine identity. The BLAST program is publicly available from NCBI and other sources (e.g., BLAST Manual, Altschul et al., NCBI NLM NIH, Bethesda, Md. 20894). These software programs match similar sequences by assigning degrees of homology to various substitutions, deletions, and other modifications. Conservative substitutions for amino acid comparisons typically include substitutions within the following groups: glycine, alanine, valine, isoleucine, leucine; aspartic acid, glutamic acid, asparagine, glutamine; serine, threonine; lysine, arginine; and phenylalanine, tyrosine.

By the term “symptoms of cancer” is meant one or more (e.g., one, two, three, four, or five) of the physical manifestations of cancer. Non-limiting examples of symptoms of cancer include blood in urine, pain or burning upon urination, cloudy urine, pain in bone, fractures in bones, fatigue, weight loss, repeated infections, nausea, vomiting, constipation, numbness in the legs, bruising, dizziness, drowsiness, abnormal eye movements, changes in vision, changes in speech, headaches, thickening of a tissue, rectal bleeding, abdominal cramps, loss of appetite, fever, enlarged lymphnodes, persistent cough, blood in sputum, lung congestion, itchy skin, lumps in skin, abdominal swelling, vaginal bleeding, jaundice, heartburn, indigestion, cell proliferation, and loss of regulation of controlled cell death.

By the term “TIA-1 related protein” or “TIAR” is a protein substantially identical to any one of NCBI Accession Nos. NP_{—}003243.1 (SEQ ID NO: 87), NP_{—}001029097.1 (SEQ ID NO: 88), and AAA36384.1 (SEQ ID NO: 89), or a nucleic acid encoding a protein substantially identical to any one of NCBI Accession Nos. NP_{—}003243.1 (SEQ ID NO: 87), NP_{—}001029097.1 (SEQ ID NO: 88), and AAA36384.1 (SEQ ID NO: 89).

By the term “phosphorylated TIAR” is meant an hsp27 protein that has been phosphorylated.

By “treating” a disease, disorder, or condition is meant delaying an initial or subsequent occurrence of a disease, disorder, or condition; increasing the disease-free survival time between the disappearance of a condition and its reoccurrence; stabilizing or reducing one or more (e.g., two, three, four, or five) adverse symptom(s) associated with a condition; or inhibiting, slowing, or stabilizing the progression of a condition. The term “treating” also includes reducing (e.g., by at least 5%, 10%, 15%, 20%, 25%, 30%, 35%, 40%, 45%, 50%, 55%, 60%, 65%, 70%, 75%, 80%, 85%, 90%, or 95% the severity or duration of one or more (e.g., one, two, three, four, or five) symptoms of a disease (e.g., cancer) in a patient. Desirably, at least 20%, 40%, 60%, 80%, 90%, or 95% of the treated subjects have a complete remission in which all evidence of the disease disappears. In another desirable embodiment, the length of time a patient survives after being diagnosed with a condition and treated using the methods of the invention is at least 20%, 40%, 60%, 80%, 100%, 200%, or even 500% greater than (i) the average amount of time an untreated patient survives or (ii) the average amount of time a patient treated with another therapy survives.

Other features and advantages of the invention will be apparent from the following description of the desirable embodiments thereof, and from the claims.

### BRIEF DESCRIPTION OF THE DRAWINGS

FIG. 1 shows that MK2 and Chk1 control temporally distinct components of the cell-cycle checkpoint response. U2OS cells were infected with lentiviruses delivering luciferase-, MK2-, or Chk1-specific shRNAs (FIG. 1A). The ability of these cells to engage and maintain a functional cell-cycle checkpoint following genotoxic stress was analyzed using a FACS-based nocodazole trap experiment. Knockdown of MK2 or Chk1 did not grossly affect the cell-cycle distribution of untreated cells (top panel). In response to 1 mM doxorubicin treatment for 1 hour, luciferase control shRNA-expressing cells mounted an intra-S and G2/M checkpoint response that remained stable for the 30-hour course of the experiment, as evidenced by the accumulation of a largely pHH3-negative 4N population (bottom panel, left, and FIG. 1B). MK2 shRNA-expressing cells initiated a functional intra-S, G2/M cell-cycle checkpoint that remained intact for at least 18 hours. At the 24-hour measurement, this checkpoint response started to decline with an increasing number of pHH3-positive cells showing a 4N DNA content, reflecting mitotically trapped cells (bottom panel, middle, and FIG. 1B). In contrast to the MK2 knockdown cells, which retained the ability to initiate a functional checkpoint response, cells that were depleted of Chk1 failed to initiate or maintain a checkpoint response within 12 hours following doxorubicin treatment (bottom panel, right, and FIG. 1B). FIG. 1B is a quantification of pHH3 staining of the samples shown in FIG. 1A (n=7 experiments, mean values are shown with error bars indicating standard deviation).

FIG. 2 shows that MK2 and Chk1 localize to distinct subcellular compartments following DNA damage-mediated activation. In FIG. 2A, GFP, GFP.MK2, and GFP.Chk1 fusion constructs were expressed in U2OS cells. Following treatment with 10 mM doxorubicin, the same set of cells was imaged using real-time imaging. GFP-MK2 relocalized to the cytoplasm within 1 hour following addition of doxorubicin and remained largely cytoplasmic for 24 hours following genotoxic stress (top panel). GFP.Chk1 remained largely nuclear through the 24-hour course of the experiment (middle panel). Unfused GFP localized diffusely throughout the cytoplasm and the nucleus. In FIG. 2B, the GFP.MK2 fusion protein is activated with the same kinetics as endogenous wild-type MK2. Stably transfected U2OS cells were either mock-treated or exposed to 10 mM doxorubicin as indicated. Following treatment, cells were lysed, and proteins were separated on SDS-PAGE and visualized by immunoblot. MK2 activity was monitored with phospho-specific antibodies detecting p38-mediated activation/phosphorylation of Thr-334, located between the kinase domain and the C-terminal regulatory domain. FIG. 2C shows that the GFP.Chk1 fusion protein is activated with the same kinetics as endogenous wild-type Chk1. Cells were treated as in FIG. 2B, and ATR-dependent phosphorylation on Ser-345 in the C-terminal regulatory region was monitored using immunoblotting. In response to doxorubicin, endogenously expressed MK2 and Chk1 show a biochemical relocalization pattern that is identical to their exogenously expressed GFP-fused counterparts, as shown in FIG. 2D. Nuclear and cytoplasmic fractions were isolated using hypotonic lysis, and MK2 and Chk1 protein levels were determined using immunoblotting. Staining for tubulin (a cytoplasmic marker) and histone H1 (a nuclear marker) was performed to assess the purity of the isolated fractions. Indirect immunofluorescence staining was performed to verify the subcellular localization of endogenous MK2 and Chk1 in response to doxorubicin. Hoechst stain was used as a counterstain to provide a nuclear reference point. As shown in FIG. 2E, MK2 was predominantly localized in the cytoplasmic compartment 6 hours following doxorubicin exposure (top panel), while Chk1 remained nuclear (bottom panel). The doxorubicin-induced cytoplasmic relocalization of MK2 depends on a caffeine-sensitive kinase(s) but is independent of Chk1, as shown in FIG. 2F. U2OS cells were either left untreated or incubated with 10 mM doxorubicin for 6 hours, and the subcellular localization of MK2 was assessed by immunofluorescence as in FIG. 2E. Doxorubicin treatment induced a robust translocation from the nucleus to the cytoplasm (upper two panels). This relocalization was completely prevented when cells were pretreated with 10 mM caffeine, 30 minutes prior to doxorubicin application (middle panel). Inhibition of Chk1 with AZD-7762 (200 nM) or PF-477736 (5 mM) 30 minutes prior to doxorubicin failed to prevent cytoplasmic accumulation of MK2 (lower two panels). FIG. 2G shows that MK2 is activated in human tumor samples. Sections from human squamous cell head and neck cancer (T) and the surrounding stroma (S) were stained with antibodies against total MK2 (left panel), the activated/phosphorylated form of MK2 (middle panel), and its downstream substrate phospho-hsp27 (right panel). These tumors show spontaneous DNA damage as indicated by positive gH2AX staining (right panel inset), which correlates with MK2 activation and cytoplasmic accumulation of MK2 (left panel inset, arrowheads). In contrast, the stroma shows predominantly nuclear staining of MK2 (left panel inset, arrows) and minimal phospho-MK2 and phospho-hsp27 staining.

FIG. 3 shows that the checkpoint response following doxorubicin requires early nuclear and late cytoplasmic basophilic protein kinase activity. MK2 and Chk1 localization mutants were generated as indicated. Live-cell images obtained before and 24 hr following treatment with 10 mM doxorubicin are shown below each construct in FIG. 3A. Of note, inactivation of the NES in MK2 results in a mutant with abolished ability to localize to the cytoplasm following genotoxic stress (FIG. 3A iii). Fusion of the MK2 NES between GFP and Chk1 produces a Chk1 mutant that is localized primarily to the cytoplasm (FIG. 3A iv). FIGS. 3B and 3C show a functional assessment of the ability of the localization mutants to establish and maintain cell-cycle checkpoints. U2OS cells were infected with lentiviruses expressing luciferase control shRNA, MK2-specific shRNA, or shRNA targeting Chk1. Knockdown cells were complemented with the localization mutants as indicated. Cells were treated with doxorubicin in a 30-hour nocodazole trap experiment, and cell-cycle profiles were assessed by FACS using DNA content profiles (FIG. 3B) and phosphohistone H3 staining (FIG. 3C). Loss of nuclear Chk1 could be functionally compensated by expression of the GFP.MK2.DNES mutant that was relocalized to the nucleus, while loss of cytoplasmic MK2 could be rescued by expression of the GFP.NES.Chk1 mutant that was relocalized to the cytoplasm. Mean values are shown with error bars indicating standard deviation.

FIG. 4 shows that MK2 is essential to stabilize Gadd45α mRNA and protein levels following genotoxic stress. FIG. 4A shows that the loss of MK2 precludes doxorubicin-induced Gadd45α mRNA and protein upregulation. HeLa cells were infected with lentiviruses expressing luciferase control or MK2-specific shRNA. Cells were treated with 10 mM doxorubicin, and Gadd45α mRNA levels were examined by RT-PCR 18 hours later. Control cells robustly induced Gadd45α after doxorubicin exposure, while MK2-depleted cells failed to upregulate Gadd45α mRNA and protein in response to doxorubicin (left and middle panel). Of note, coexpression of the GFP.NES.Chk1 mutant that relocalized to the cytoplasm rescued the MK2 RNAi phenotype (right panel). FIG. 4B shows that Gadd45α depletion in functionally p53-deficient HeLa cells prevents the engagement of a functional intra-S, G2/M checkpoint following doxorubicin treatment. HeLa cells expressing luciferase control shRNA or Gadd45α-specific hairpins were treated with doxorubicin (10 mM) in a 30-hour nocodazole trap experiment, and cell-cycle profiles were assessed by FACS. Control cells mounted a robust intra-S, G2/M arrest in response to doxorubicin, as evidenced by an accumulation of 4N cells (monitored by PI staining) and a lack of pHH3 staining. In contrast, ˜23% of Gadd45α-depleted cells entered mitosis throughout the 30-hour course of the experiment, indicating a bypass of the doxorubicin-induced cell-cycle arrest in these cells. Mean values are shown with error bars indicating standard deviation. FIG. 4C shows that fusion of the Gadd45α mRNA 3′ UTR to GFP confers MK2-dependent sensitivity to genotoxic stress to GFP protein expression. HeLa cells expressing luciferase control shRNA or MK2-specific hairpins were cotransfected with vectors encoding unfused eGFP or eGFP fused to the Gadd45α mRNA3′UTR. In these experiments, the GFP-3′ UTR protein is ˜3 kD smaller than eGFP due to a deletion of 23 amino acids at the C-terminus. Cells were mock treated or exposed to cisplatin (10 mM), doxorubicin (1 mM), or UV (20 J/m²), harvested 36 hours later, and GFP expression levels monitored by immunoblot. Expression levels of unfused GFP did not change following genotoxic stress; however, fusion of the Gadd45a 3′ UTR to GFP resulted in repression of GFP expression that could be relieved upon genotoxic stress in a MK2-dependent manner. The right panel schematically depicts the endogenous Gadd45a transcript (top), the GFP-3′ UTR fusion, and unfused GFP constructs. FIG. 4D shows that the relative GFP expression levels as shown in FIG. 4C were quantified from three independent experiments using ImageQuant software. Mean values are shown with error bars indicating standard deviation. Note the expanded y-axis scale in panels 2-4.

FIG. 5 shows that doxorubicin triggers MK2-dependent complex formation between hnRNPA0 and the GADD45a mRNA 3′ UTR, resulting in GADD45a mRNA stabilization and increased GADD45a protein levels. FIG. 5A shows immunoprecipitation followed by Western blotting for the RBPs that were investigated. HeLa cells were either treated with doxorubicin (1 mM) for 12 hours or left untreated, lysed, and the binding of endogenous ARE-binding RBPs (HuR, TIAR, TTP, and hnRNPA0) to Gadd45a mRNA assessed using RNA-IP and shown in FIG. 5B. FIG. 5C shows that hnRNPA0 interacts with the Gadd45a 3′ UTR following genotoxic stress. HeLa cells were cotransfected with HA-tagged hnRNPA0 and either GFP fused to the Gadd45α 3′ UTR or unfused GFP. Cells were treated with doxorubicin (10 mM) or vehicle for 12 hours, lysed, and HA.hnRNPA0 was immunoprecipitated followed by GFP RT-PCR. hnRNPA0 strongly bound to Gadd45a 3′ UTR-fused GFP mRNA following doxorubicin. However, no interaction between hnRNPA0 and unfused GFP mRNA was detected, indicating that hnRNPA0 directly binds to the 3′ UTR of Gadd45a mRNA. FIG. 5D shows that hnRNPA0 depletion in functionally p53-deficient HeLa cells prevents the engagement of a functional intra-S, G2/M checkpoint following doxorubicin. HeLa cells expressing luciferase control shRNA or hnRNPA0-specific hairpins were treated with 10 mM doxorubicin in a 30-hour nocodazole trap experiment, and cell-cycle profiles were assessed by FACS. Control cells mounted a robust intra-S, G2/M arrest in response to doxorubicin, as evidenced by an accumulation of 4N cells (monitored by PI staining), which were largely staining negative for pHH3. In contrast, ˜15% of hnRNPA0-depleted cells entered mitosis throughout the 30-hour course of the experiment, indicating a bypass of the doxorubicin-induced cell-cycle arrest in these cells. Mean values are shown with error bars indicating standard deviation. In FIG. 5E, shown are in vitro kinase assays with bacterially purified recombinant MK2 and GST.hnRNPA0 wild-type or hnRNPA0 in which Ser-84 was mutated to alanine. GST served as a control. Following completion of the kinase assay, reaction mixtures were separated on SDS-PAGE and ³²P incorporation was visualized by autoradiography. GST.hnRNPA0 wild-type was readily phosphorylated by MK2 in vitro, while mutation of Ser-84 to alanine completely abolished hnRNPA0 phosphorylation. FIG. 5F shows that MK2-mediated hnRNPA0 phosphorylation on Ser-84 is essential for hnRNPA0 binding to Gadd45a mRNA. HeLa cells expressing luciferase control shRNA or MK2-specific shRNA were transfected with HA-tagged hnRNPA0 wild-type or the Ser-84 to alanine mutant. Cells were treated with doxorubicin (10 mM) or vehicle, lysed 12 hours later, and hnRNPA0 immunoprecipitated with anti-HA-antibodies. While HA.hnRNPA0 readily coprecipitaed with Gadd45a mRNA following genotoxic stress in control cells, the Ser-84 to alanine mutant failed to interact with Gadd45a mRNA (left panel). This interaction was MK2-dependent, since hnRNPA0:Gadd45a mRNA complex formation was abolished in MK2-depleted cells (middle panel). Loss of MK2 could be rescued by expression of the activatable, cytoplasmic Chk1 mutant. FIG. 5G shows that reduced binding of Gadd45a mRNA by TIAR following genotoxic stress depends on p38 activity. HeLa cells were treated with the p38 inhibitor SB203580 (10 mM) or vehicle 1-hour before treatment with doxorubicin as described in FIG. 5F. TIAR was immunoprecipitated followed by Gadd45a RT-PCR. TIAR binding to the Gadd45a mRNA that was abolished following genotoxic stress could be restored by inhibition of p38. FIG. 5H shows in vitro kinase assays with bacterially purified recombinant His.MK2 or His.p38 and GST.TIAR. GST served as a control and GST.Hsp25-peptide (AS 71-100) served as a positive control for MK2. Following completion of the kinase assay, reaction mixtures were separated on SDS-PAGE, and ³²P incorporation was visualized by autoradiography. GST.TIAR was readily phosphorylated by p38 in vitro after 20 minutes, but it was not phosphorylated by MK2.

FIG. 6 shows that MK2 directly phosphorylates PARN on Ser-557 following genotoxic stress. FIG. 6A shows data from in vitro kinase assays using bacterially purified recombinant MK2 and 6×His-tagged PARN wild-type or a PARN mutant in which Ser-557 was mutated to alanine. Following completion of the kinase assay, reaction mixtures were separated on SDS-PAGE and ³²P incorporation was visualized by autoradiography. Equal loading was confirmed by Coomassie staining. The top panel shows a schematic representation of the modular domain structure of PARN. Ser-557 lies within an optimal MK2 consensus phosphorylation motif located C-terminal to the RNA recognition motif (RRM). FIG. 6b shows that MK2 mediates doxorubicin-induced phosphorylation of PARN on Ser-557 within cells. U2OS cells were infected with lentiviruses expressing luciferase or an MK2-specific shRNA. Following selection, cells were treated with 10 mM doxorubicin for 4 hours and endogenous PARN was affinity-purified from cell lysates. The immunoprecipitated material was analyzed by mass spectrometry. Only nonphosphorylated Ser-557 PARN peptides could be detected in untreated U2OS cells expressing the luciferase control shRNA (shLuci, co). In contrast, Ser-557-phosphorylated peptides were readily detected when luciferase control cells were exposed to doxorubicin (shLuci, dox). DNA damage-induced phosphorylation of PARN on Ser-557 was completely abolished in MK2-depleted cells (shMK2 co and dox panels). FIGS. 6C and 6D show that PARN Ser-557 phosphorylation is critical for maintenance of a doxorubicin-induced cell-cycle arrest. HeLa cells were infected with lentiviruses expressing packaged from empty transfer vector or PARN shRNA-expressing vectors. PARN shRNA-expressing cells were also cotransfected with shRNA-resistant PARN wild-type or a Ser-557 to alanine mutant. Cells were treated with 0.1 mM doxorubicin for 1 hour, and cell-cycle profiles (phosphohistone H3 and DNA content) were assessed in a nocodazole trap experiment using FACS to monitor mitotic entry and cell-cycle progression. After 24 hours, 5 mM caffeine was added to abrogate checkpoint signaling and analyze the ability of damaged cells to exit the checkpoint. Empty vector, PARN shRNA, and PARN shRNA-expressing cells that were complemented with shRNA-resistant wild-type PARN showed the induction of a stable cell-cycle arrest, as evidenced by an accumulation of S and G2/pHH3-negative cells. PARN shRNA-expressing cells that were coexpressing the shRNA-resistant, nonphosphorylatable PARN S557A mutant failed to maintain a functional cell-cycle arrest, indicated by the accumulation of ˜12% of pHH3-positive cells at 24 hours following addition of low-dose doxorubicin. Mean values are shown with error bars indicating standard deviation. FIG. 6E shows that PARN Ser-557 is critical for long-lasting expression of Gadd45a mRNA and protein following doxorubicin-induced genotoxic stress. Cells were transfected and treated with doxorubicin as in FIG. 6C. Gadd45a mRNA levels were monitored by RT-PCR and protein levels were assessed by immunoblotting. Of note, cells expressing the nonphosphorylatable PARN Ser-557A mutant showed upregulation of Gadd45a mRNA and protein levels at 12 hours, but could not sustain the stabilization of this inherently unstable mRNA for longer times. This loss of Gadd45a expression at 24 hours coincided with the premature cell-cycle checkpoint collapse shown in FIG. 6C.

FIG. 7 shows that Gadd45α is required to maintain long-term MK2 activity and prevent premature checkpoint collapse following genotoxic stress. FIG. 7A shows that Gadd45a interacts with p38. Cells were transfected with HA-tagged Gadd45a or HA-tagged 14-3-3z as a negative control. Lysates and anti-HA IPs were analyzed by SDS-PAGE and Western blotting using anti-p38 and anti-HA-antibodies. FIG. 7B shows that Gadd45a is required to maintain long-term MK2 activity following doxorubicin-induced DNA damage. HeLa cells were infected with lentiviruses expressing either luciferase control shRNA or MK2-specific hairpins, treated with 1 mM doxorubicin for the indicated times, lysed, and proteins separated on SDS-PAGE. MK2 and Gadd45a levels were monitored by immunoblot; β-actin staining served as a loading control. Both Gadd45a and control shRNA-expressing cells showed a robust induction of MK2 activity 18 hours following doxorubicin treatment (monitored by phosphorylation-induced gel shift to a slower migrating isoform on SDS-PAGE). However, control cells maintained MK2 activity for at least 30 hours, while Gadd45a-depleted cells were unable to maintain MK2 activity for more than 18 hours. FIG. 7C shows that the activation-induced MK2 gel shift is blocked by the p38 inhibitor SB203580. Doxorubicin treatment (10 mM) of HeLa cells induces a phosphorylation-dependent gel shift of MK2 (middle lane). Pretreatment of HeLa cells with 10 mM of the p38 inhibitor SB203580 abolished the activation-induced MK2 gel shift (right lane). FIG. 7D shows a simplified model depicting the early, Chk1-dependent nuclear checkpoint (left box) and the late MK2-dependent cytoplasmic checkpoint (right box). Dashed arrows between ATM/ATR and p38/MK2 indicate intermediate steps that are not shown. The MK2-mediated cytoplasmic checkpoint is sustained through a positive feedback loop. Following nuclear activation, the p38/MK2 signaling complex relocalizes to the cytoplasm through a Crm1-dependent transport mechanism. MK2-mediated hnRNPA0 and PARN phosphorylation, as well as p38-dependent TIAR phosphorylation, are required to stabilize Gadd45α mRNA, resulting in increased Gadd45a protein levels. Gadd45a itself is then required to maintain MK2 activity in the cytoplasm.

FIG. 8 shows that MK2 is required for the retention of cdc25B and cdc25C in the cytoplasm at late stages of the cell-cycle checkpoint response to prevent inappropriate mitotic re-entry. FIG. 8A shows that HeLa cells were infected with retroviruses encoding GFP-fused cdc25B (upper panels) or cdc25C (lower panels). GFP.cdc25B/C-expressing cells were subsequently infected with lentiviruses expressing luciferase-, MK2-, or Chk1-specific shRNA; treated with 0.1 mM doxorubicin for 1 hour; and GFP localization monitored by live-cell imaging. Control cells mounted a cell-cycle checkpoint response and resumed mitotic cell division after ˜30 hours with the production of two intact daughter cells (top panels and top panel in FIG. 8B). MK2-depleted cells initiated a checkpoint response, which collapsed after ˜24 hours, invariably followed by a catastrophic mitotic event (middle panels and FIG. 8B, middle panel). Chk1-depleted cells entered a premature, catastrophic mitotic event at ˜16 hours following doxorubicin (middle panels and FIG. 8B, middle panel). FIG. 8B shows a detailed view of the mitotic events in the three different GFP.cdc25B-labeled cell lines. FIG. 8C shows quantitative analysis of the data shown in FIGS. 8A and 8B. Data are shown as box and whisker plots and represent 18 independent experiments for each cell line. The time of the first appearance of a mitotic figure was recorded. Asterisk indicates statistical significance. Mean values are shown with error bars indicating standard deviation.

### DETAILED DESCRIPTION

In view of the characterization of the MK2 signaling pathway and its importance for maintenance of the G₂/M checking point, the invention provides methods of reducing the severity of one or more symptoms of cancer in a patient, methods of identifying a cancer patient that may selectively benefit from the administration of one or more MK2 inhibitor(s) or the combination of one or more MK2 inhibitor(s) and one or more chemotherapeutic agent(s), methods of identifying a cancer patient that may selectively benefit from the administration of one or more chemotherapeutic agent(s), methods of diagnosing a chemotherapy-resistant cancer or a chemotherapy-sensitive cancer cell in a patient, and kits for diagnosing a chemotherapy-resistant or chemotherapy-sensitive cancer in a patient.

### Methods of Treating Cancer

The invention provides methods for treating cancer that include a step for determining the activation or inactivation of the MK2 signaling pathway in cancer cell(s) from the patient and, optionally, determining the inactivation of the p53 pathway in cancer cell(s) from the patient. In view of this determination(s), the patient is differentially administered one or more MK2 inhibitor(s) or the combination of one or more MK2 inhibitor(s) and one or more chemotherapeutic agent(s) (patients having cancer cells with activated MK2 signaling pathway, such a patient having cancer cells with an activated MK2 signaling pathway and an inactivated p53 pathway), or administered one or more chemotherapeutic agent(s) (patients having cancer cells with inactivated MK2 signaling pathway, such as, patients having an inactivated MK2 signaling pathway and an inactivated p53 pathway). The invention further provides method of treating a cancer patient diagnosed as having a chemotherapy-resistant or a chemotherapy-sensitive cancer using the diagnostic methods provided herein (e.g., by a diagnostic or clinical laboratory), where a patient diagnosed as having a chemotherapy-resistant cancer is administered one or more MK2 inhibitor(s) and a patient diagnosed as having a chemotherapy-sensitive cancer is administered one or more chemotherapeutic agent(s).

Measurement of a MK2 Pathway Activation or Inactivation

Applicants have characterized the MK2 signaling pathway that is required for the maintenance of the G₂/M checkpoint following genotoxic stress. Following phosphorylation by p38 in the nucleus, MK2 translocates to the cytoplasm. Thus, one indication of an activated MK2 pathway is an increase of MK2 protein in the cytoplasm and/or a decrease of MK2 protein in the nucleus. A further indication of activated MK2 signaling pathway is an increase in total MK2 protein phosphorylation (e.g., an increase in the levels of phosphorylated MK2 protein in the cytoplasm or nucleus).

In the cytoplasm, phosphorylated MK2 protein is herein implicated as having a role in the phosphorylation of a number of regulatory proteins: cdc25B (e.g., phosphorylation at serine 323), cdc25C (e.g., phosphorylation at serine 216), TIAR, hnRNPA0 (e.g., phosphorylation at serine 84), PARN (e.g., phosphorylation at serine 557), and hsp-27 (e.g., phosphorylation at serine 15, serine 78, and/or serine 82). Thus, an increase in the phosphorylation of one or more of these substrate proteins may further indicate an activated MK2 signaling pathway.

Finally, the phosphorylation of several of the substrate proteins TIAR, hnRNPA0, and PARN has been indicated to result in the stabilization of the Gadd45a mRNA and thus, results in an increase in Gadd45a mRNA and protein levels in the cell. Thus, increased levels of Gadd45a mRNA and/or protein also indicate an activated MK2 pathway.

In sum, an activated MK2 signaling pathway may be indicated by one or more (e.g., two, three, four, five, or six) of the following features: increased (e.g., by at least 10%, 20%, 30%, 40%, 50%, 60%, 70%, 80%, or 90%) MK2 protein in the cytoplasm, decreased (e.g., by at least 10%, 20%, 30%, 40%, 50%, 60%, 70%, 80%, or 90%) MK2 protein in the nucleus, increased (e.g., by at least 10%, 20%, 30%, 40%, 50%, 60%, 70%, 80%, or 90%) levels of total MK2 protein phosphorylation, increased (e.g., by at least 10%, 20%, 30%, 40%, 50%, 60%, 70%, 80%, or 90%) levels of phosphorylated MK2 protein in the cytoplasm or nucleus, increased (by at least 10%, 20%, 30%, 40%, 50%, 60%, 70%, 80%, or 90%) levels of phosphorylated cdc25B (e.g., phosphorylation at serine 323), increased (by at least 10%, 20%, 30%, 40%, 50%. 60%, 70%, 80%, or 90%) levels of phosphorylated cdc25C (e.g., phosphorylation at serine 216), increased (by at least 10%, 20%, 30%, 40%, 50%, 60%, 70%, 80%, or 90%) levels of phosphorylated TIAR, increased (by at least 10%, 20%, 30%, 40%, 50%, 60%, 70%, 80%, or 90%) levels of phosphorylated hnRNPA0 (e.g., phosphorylation at serine 84), increased (by at least 10%, 20%, 30%, 40%, 50%, 60%, 70%, 80%, or 90%) levels of phosphorylated PARN (e.g., phosphorylation at serine 557), and increased (by at least 10%, 20%, 30%, 40%, 50%, 60%, 70%, 80%, or 90%) levels of phosphorylated hsp-27 (e.g., phosphorylation at serine 15, serine 78, and/or serine 82).

Conversely, an inactivated MK2 signaling pathway may be indicated by one or more (e.g., two, three, four, five, or six) of the following features: decreased (by at least 10%, 20%, 30%, 40%, 50%, 60%, 70%, 80%, or 90%) levels of MK2 protein in the cytoplasm, increased (by at least 10%, 20%, 30%, 40%, 50%, 60%, 70%, 80%, or 90%) levels of MK2 protein in the nucleus, decreased (by at least 10%, 20%, 30%, 40%, 50%, 60%, 70%, 80%, or 90%) levels of total MK2 protein phosphorylation, decreased (by at least 10%, 20%, 30%, 40%, 50%, 60%, 70%, 80%, or 90%) levels of phosphorylated MK2 in the cytoplasm or nucleus, decreased (by at least 10%, 20%, 30%, 40%, 50%, 60%, 70%, 80%, or 90%) levels of phosphorylated cdc25B (e.g., phosphorylation at serine 323), decreased (by at least 10%, 20%, 30%, 40%, 50%, 60%, 70%, 80%, or 90%) levels of phosphorylated cdc25C (e.g., phosphorylation at serine 216), decreased (by at least 10%, 20%, 30%, 40%, 50%, 60%, 70%, 80%, or 90%) levels of phosphorylated TIAR, decreased (by at least 10%, 20%, 30%, 40%, 50%, 60%, 70%, 80%, or 90%) levels of phosphorylated hnRNPA0 (e.g., phosphorylation at serine 84), decreased (by at least 10%, 20%, 30%, 40%, 50%, 60%, 70%, 80%, or 90%) levels of phosphorylated PARN (e.g., phosphorylation at serine 557), and decreased (by at least 10%, 20%, 30%, 40%, 50%, 60%, 70%, 80%, or 90%) levels of phosphorylated hsp-27 (e.g., phosphorylation at serine 15, serine 78, and/or serine 82). Various methods for the determining the activation or inactivation of an MK2 signaling pathway are known in the art and non-limiting examples are described below.

The amount of MK2 protein in the cytoplasm or the nucleus of a cell may be measured using an antibody that is specific for MK2. In one example, a cell may be differentially lysed to prepare a separate nuclear extract and/or cytosolic lysates. Immunoblotting may be performed using an MK2 antibody to determine the levels of MK2 protein found in the cytoplasm and/or the nucleus. Alternatively, the relative amount of MK2 in the nucleus and cytoplasm may be measured by immunofluorescence microscopy using labeled MK2 antibodies (e.g., fluorescently-labeled antibodies). The relative increase in MK2 protein levels in the cytoplasm or the relative decrease in MK2 protein levels in the nucleus of a cancer cell may be compared to a non-cancerous cell (e.g., a non-cancerous cell from the patient) or a cell that has not been exposed to a genotoxic agent (e.g., a DNA-damaging or chemotherapeutic agent). The relative decrease in MK2 protein levels in the cytoplasm or the relative increase in MK2 protein levels in the nucleus of a cancer cell may be compared to a cancer cell or a cell that has been exposed to a genotoxic agent (e.g., a DNA-damaging or chemotherapeutic agent).

The total amount of phosphorylated MK2 protein may be measured using methods known in the art. For example, such techniques often utilize an antibody that specifically recognizes the phosphorylated form of MK2 protein. In one example, a cellular lysate from cancer cells may be prepared and immunoblotted using an antibody that specifically binds phosphorylated MK2. Alternatively, the total amount of phosphorylated MK2 present in a cell may be measured using immunofluorescent microscopy or fluorescence-assisted cell sorting (FACS) that utilizes a fluorescently-labeled antibody that specifically binds to phosphorylated MK2. Similarly, the amount of phosphorylated MK2 in the cytoplasm or nucleus may be measured using antibodies that specifically bind to the phosphorylated form of MK2. For example, a cytosolic extract or nuclear extract may be prepared from cancer cells using differential lysis and the prepared extract immunoblotted using an antibody that specifically binds to phosphorylated MK2. Similarly, immunofluorescence microscopy may be performed using a fluorescently-labeled antibody that specifically binds to phosphorylated MK2 to measure the amount of phosphorylated MK2 that is present in a cancer cell (e.g., the amount of phosphorylated MK2 protein that is present in the cytosol or nucleus).

The relative increase in total phosphorylated MK2 protein or the relative increase in phosphorylated MK2 protein in the cytoplasm or nucleus of a cancer cell(s) may be compared to a non-cancerous cell (e.g., a non-cancerous cell from the patient) or a cell that has not been exposed to a genotoxic agent (e.g., a DNA-damaging or chemotherapeutic agent). The relative decrease in total phosphorylated MK2 protein or the relative decrease in phosphorylated MK2 protein in the cytoplasm or nucleus of a cancer cell(s) may be compared to a cancer cell or a cell that has been exposed to a genotoxic agent (e.g., a DNA-damaging or chemotherapeutic agent).

The phosphorylation of cdc25B (e.g., phosphorylation at serine 323), cdc25C (e.g., phosphorylation at serine 216), TIAR, hnRNPA0 (e.g., phosphorylation at serine 84), PARN (e.g., phosphorylation at serine 557), and hsp-27 (e.g., phosphorylation at serine 15, serine 78, and/or serine 82) may also be measured using antibodies that specifically recognize the phosphorylated forms of these target proteins. As described above, antibodies that specifically bind to the phosphorylated form of each target protein may be used to measure the total amount of the phosphorylated target protein present in a cell or a cell extract. For example, these phosphorylation-specific antibodies may be used to perform immunoblotting on extracts prepared from cancer cell(s). Such methods may be automated or performed using protein chip assays. Alternatively, the phosphosphorylation-specific antibodies may be fluorescently-labeled and used in FACS analysis or immunofluorescent microscopy to measure the total amount of the target phosphorylated protein present in a cancer cell. The relative increase in phosphorylated cdc25B, cdc25C, TIAR, hnRNPA0, PARN, and/or hsp-27 may be compared to a non-cancerous cell (e.g., a non-cancerous cell from the patient) or a cell that has not been exposed to a genotoxic agent (e.g., a DNA-damaging or chemotherapeutic agent). The relative decrease in phosphorylated cdc25B, cdc25C, TIAR, hnRNPA0, PARN, and/or hsp-27 may be compared to a cancer cell or a cell that has been exposed to a genotoxic agent (e.g., a DNA-damaging or chemotherapeutic agent).

Finally, the total amount of Gadd45a mRNA or protein may be measured in the cell using molecular biology techniques known in the art. For example, the levels of Gadd45a mRNA may be measured using any nucleic acid that is complementary to a contiguous sequence present in Gadd45a mRNA. For example, the amount of Gadd45a mRNA may be detected using fluorescent in situ hybridization (FISH) using such an antisense nucleic acid. Gadd45a mRNA levels may also be measured using techniques based on polymerase chain reaction (PCR) using primers specifically designed to amplify an mRNA encoding Gadd45a protein (e.g., reverse-transcriptase PCR, real-time qPCR, or gene array technology). Gadd45a protein levels may be measured using an antibody that specific binds to Gadd45 protein. For example, immunoblotting may be performed on whole cell extract using a Gadd45 antibody. Similarly, a fluorescently-labeled Gadd45 antibody may be used to perform immunofluorescent microscopy or FACS analysis on cancer cells. The relative increase in Gadd45a protein or mRNA levels may be compared to a non-cancerous cell (e.g., a non-cancerous cell from the patient) or a cell that has not been exposed to a genotoxic agent (e.g., a DNA-damaging or chemotherapeutic agent). The relative decrease in Gadd45a protein or mRNA levels may be compared to a cancer cell or a cell that has been exposed to a genotoxic agent (e.g., a DNA-damaging or chemotherapeutic agent).

Measurement of p53 Pathway Inactivation

The p53 pathway has been shown to mediate cell cycle arrest following genotoxic stress. Specifically, phosphorylated p53 has DNA-binding activity and mediates the induction of p21 gene expression in the cell. In several cancer cells, a mutation or truncation (e.g., one or more amino acid substitutions, deletions, and/or additions) of the p53 protein is observed that results in decreased activity or expression. For example, a mutation or truncation of the p53 protein may result in a decrease (e.g., at least a 10%, 20%, 30%, 40%, 50%, 60%, 70%, 80%, 90%, or 95% decrease) in DNA-binding activity, a decrease (e.g., at least a 10%, 20%, 30%, 40%, 50%, 60%, 70%, 80%, 90%, or 95% decrease) in the ability to induce p21 induction, or a decrease (e.g., at least a 10%, 20%, 30%, 40%, 50%, 60%, 70%, 80%, 90%, or 95% decrease) in the ability to mediate cell cycle arrest in response to genotoxic stress (e.g., cell cycle arrest in response to a DNA-damaging chemotherapeutic agent). Similarly, inactivation of p53 may occur in the cell by way of a gene translocation event which results in the formation of a p53 fusion protein that has a decrease (e.g., at least 10%, 20%, 30%, 40%, 50%, 60%, 70%, 80%, 90%, or 95% decrease) in the ability to bind DNA, a decrease (e.g., at least a 10%, 20%, 30%, 40%, 50%, 60%, 70%, 80%, 90%, or 95% decrease) in the ability to induce p21 induction, or a decrease (e.g., at least a10%, 20%, 30%, 40%, 50%, 60%, 70%, 80%, 90%, or 95% decrease) in the ability to mediate cell cycle arrest in response to genotoxic stress. Further, inactivation of p53 may occur by a loss of heterozygosity mutation, where the mutation in a second allele of the p53 gene occurs following a mutation in the first allele of the p53 gene. Thus, loss of p53 signaling may be indicated by one or more (e.g., two, three, or four) of the following features: decreased (e.g., by at least 10%, 20%, 30%, 40%, 50%, 60%, 70%, 80%, or 90%) p53 mRNA or protein levels, expression of a mutant or truncated p53 with decreased (e.g., by at least 10%, 20%, 30%, 40%, 50%, 60%, 70%, 80%, or 90%) expression or activity, and decreased (by at least 10%, 20%, 30%, 40%, 50%, 60%, 70%, 80%, or 90%) p21 expression or activity. Various methods for measuring p53 pathway inactivation are known in the art and non-limiting examples are provided below.

The levels of p53 mRNA or protein may be measured using a number of molecular biology techniques known in the art. p53 mRNA may be measured using any nucleic acid that is complementary to a contiguous sequence present in p53 mRNA. For example, the amount of p53 mRNA may be detected using FISH using such an antisense nucleic acid. p53 mRNA levels may also be measured using techniques based on PCR using primers specifically designed to amplify an mRNA encoding p53 protein (e.g., reverse-transcriptase PCR, real-time qPCR, or gene array technology). p53 protein levels may be measured using an antibody that specific binds to p53 protein. For example, immunoblotting may be performed on whole cell extract using a p53 antibody. Similarly, a fluorescently-labeled p53 antibody may be used to perform immunofluorescence microscopy or FACS analysis on cancer cells. The relative decrease in p53 protein or mRNA levels may be compared to a non-cancerous cell (e.g., a non-cancerous cell from the patient). Importantly, measurements of p21 protein expression by immunoblotting, immunohistochemistry, or immunofluorescence microscopy, for example, may be used as highly sensitive assays of p53 function.

The expression of a mutant or truncated p53 protein with decreased expression or activity (e.g., decreased DNA-binding activity, ability to induce cell cycle arrest following genotoxic stress, and/or the ability to induce p21 gene expression) may be measured using molecular biology techniques known in the art. For example, mutations or truncations in p53 protein may be detected using PCR-based techniques using primers that specifically amplify the region of the p53 mRNA or gene (e.g., reverse-transcriptase PCR, real-time qPCR, or gene array technology). In addition, methods to analyze or determine the presence of a mutation in a second allele of the p53 locus may be identified using single-nucleotide polymormorphism microarray analysis.

Inactivated p53 signaling may also be observed by a decrease in p21 mRNA or protein expression in a cell (e.g., reduced induction of p21 expression following genotoxic stress). p53 mRNA may be may be measured using any nucleic acid that is complementary to a contiguous sequence present in p53 mRNA. For example, the amount of p21 mRNA may be detected using FISH using such an antisense nucleic acid. p21 mRNA levels may also be measured using techniques based on PCR using primers specifically designed to amplify an mRNA encoding p21 protein (e.g., reverse-transcriptase PCR, real-time qPCR, or gene array technology). p21 protein levels may be measured using an antibody that specific binds to p21 protein. For example, immunoblotting may be performed on whole cell extract using a p21 antibody. Similarly, a fluorescently-labeled p21 antibody may be used to perform immunofluorescence microscopy or FACS analysis on cancer cells. The relative decrease in p21 protein or mRNA levels may be compared to a non-cancerous cell (e.g., a non-cancerous cell from the patient or a non-cancerous cell exposed to a genotoxic agent, such as a DNA-damaging chemotherapeutic agent).

### MK2 Inhibitors

Any compound or pharmaceutical composition that inhibits an activity of MK2 may be useful in the methods of treatment provided by the invention. Non-limiting examples of MK2 inhibitors are described below.

Peptides

Peptides that mimic a natural peptide substrate of MK2 may decrease the extent or rate with which MK2 is able to bind to its natural substrates in vivo. Accordingly, such peptides may be used as MK2 inhibitors in the treatment methods provided by the invention. For example, a peptide (e.g., less than 50 total amino acids) containing the amino acid sequence [L/F/I]XR[Q/S/T]L[S/T][hydrophobic] (SEQ ID NO: 5) may be used as a MK2 inhibitor (e.g., a peptide containing the amino acid sequence LQRQLSI (SEQ ID NO: 6)). Additional examples of peptides that may function as MK2 inhibitors are described in U.S. Patent Application No. 2009/0010927, herein incorporated by reference.

Small Molecules

Any small molecule that inhibits MK2 (e.g., MK2 kinase activity), whether specifically or nonspecifically, may be of utility in the methods provided by the invention. Additional non-limiting examples of small molecule MK2 inhibitors are described above in the section titled “Summary of the Invention.” Others small molecule inhibitors of MK2 are described in U.S. Patent Application Publication Nos. 2004/0127492, 2005/0101623, 2005/0137220, and 2005/0143371.

Antisense Nucleic Acids

MK2 antisense nucleic acids may be also be used as MK2 inhibitors in the methods of the invention. Sequence-specific suppression of gene expression can be achieved by intracellular hybridization between mRNA and a complementary antisense species. The formation of a hybrid RNA duplex may then interfere with the processing/transport/translation and/or stability of the target MK2 mRNA. Antisense strategies may use a variety of approaches, including the use of antisense oligonucleotides and injection of antisense RNA. An exemplary approach features transfection of antisense RNA expression vectors into targeted cells. Antisense effects can be induced by control (sense) sequences; however, the extent of phenotypic changes are highly variable. Phenotypic effects induced by antisense effects are based on changes in criteria such as protein levels, protein activity measurement, and target mRNA levels.

Computer programs such as OLIGO (previously distributed by National Biosciences Inc.) may be used to select candidate nucleobase oligomers for antisense therapy based on the following criteria:
1) No more than 75% GC content, and no more than 75% AT content;
2) Preferably no nucleobase oligomers with four or more consecutive G residues (due to reported toxic effects, although one was chosen as a toxicity control);
3) No nucleobase oligomers with the ability to form stable dimers or hairpin structures; and
4) Sequences around the translation start site are a preferred region.
   In addition, accessible regions of the target mRNA may be predicted with the help of the RNA secondary structure folding program MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, 1999). Sub-optimal folds with a free energy value within 5% of the predicted most stable fold of the mRNA may be predicted using a window of 200 bases within which a residue can find a complimentary base to form a base pair bond. Open regions that do not form a base pair may be summed together with each suboptimal fold, and areas that consistently are predicted as open may be considered more accessible to the binding to nucleobase oligomers. Additional nucleobase oligomer that only partially fulfill some of the above selection criteria may also be chosen as possible candidates if they recognize a predicted open region of the target mRNA.

RNAi-Based Inhibitors

Nucleobase oligomers may be used as MK2 inhibitors in the methods of the invention. For example, double-stranded RNAs may be used to elicit RNAi-mediated knockdown of MK2 expression. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of siRNAs using a plasmid-based expression system is increasingly being used to create loss-of-function phenotypes in mammalian cells.

In one embodiment of the invention, a double-stranded RNA (dsRNA) molecule is made. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505 2002, each of which is hereby incorporated by reference.

Small hairpin RNAs consist of a stem-loop structure with optional 3′ UU-overhangs. While there may be variation, stems can range from twenty-one to thirty-one base pairs (desirably twenty-five to twenty-nine base pairs), and the loops can range from four to thirty base pairs (desirably four to twenty-three base pairs). For expression of shRNAs within cells, plasmid vectors containing, e.g., the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

Computer programs that employ rational design of oligos are useful in predicting regions of the MK2 sequence that may be targeted by RNAi. For example, see Reynolds et al., Nat. Biotechnol., 22:326-330, 2004, for a description of the Dharmacon siDESIGN tool. Table 1 lists several exemplary nucleotide sequences within MAPKAP kinase-2 that may be targeted for purposes of RNA interference. siRNA or shRNA oligos may be made corresponding to the sequences shown and including an overhang, e.g., a 3′ dTdT overhang and/or a loop.

TABLE 1
MK2 RNAi target sequences

| Sequence (5′ to 3′) | SEQ ID NO: |
|---|---|
| GACCAGGCATTCACAGAAA | 7 |
| TTGACCACTCCTTGTTATA | 8 |
| GACCACTCCTTGTTATACA | 9 |
| TGACCATCACCGAGTTTAT | 10 |
| TCACCGAGTTTATGAACCA | 11 |
| TCAAGAAGAACGCCATCAT | 12 |
| AAGCATCCGAAATCATGAA | 13 |
| AGTATCTGCATTCAATCAA | 14 |
| CTTTGACCACTCCTTGTTA | 15 |
| TTTGACCACTCCTTGTTAT | 16 |
| TACGGATCGTGGATGTGTA | 17 |
| GGACGGTGGAGAACTCTTT | 18 |
| CTTGTTATACACCGTACTA | 19 |
| GACGGTGGAGAACTCTTTA | 20 |
| GGAGAACTCTTTAGCCGAA | 21 |

Non-limiting examples of siRNA molecules that may be used as MK2 inhibitors in the methods of the invention include a nucleic acid containing the sequence of any one of CGAUGCGUGUUGACUAUGAdTdT (SEQ ID NO: 1), UCAUAGUCAACACGCA UCGdTdT (SEQ ID NO: 2), UGACCAUCACCGAGUUUAUdTdT (SEQ ID NO: 3), or AUAAACUCGGUGAUGGUCAdTdT (SEQ ID NO: 4).

Antibodies

Additional MK2 inhibitors include antibodies (e.g., human monoclonal antibodies) that specifically bind to total MK2 or phosphorylated MK2. Methods for the generation of monoclonal antibodies using hybridoma technology are known in the art. MK2-specific antibodies are desirably produced using MK2 protein sequences that do not reside within highly conserved regions, and that appear likely to be antigenic, as evaluated by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into any appropriate expression vector. For example, GST fusion proteins can be expressed in E. coli and purified using a glutathione-agarose affinity matrix. To minimize the potential for obtaining antisera that is non-specific or exhibits low-affinity binding to MK2, two or three MK2 fusion proteins may be generated for each fragment injected into a separate animal. Antisera are raised by injections in series, preferably including at least three booster injections.

In addition to intact monoclonal and polyclonal anti-MK2 protein, various genetically engineered antibodies and antibody fragments (e.g., F(ab′)2, Fab′, Fab, Fv, and sFv fragments) can be produced using standard methods. Truncated versions of monoclonal antibodies, for example, can be produced by recombinant methods in which plasmids are generated that express the desired monoclonal antibody fragment(s) in a suitable host. Ladner (U.S. Pat. Nos. 4,946,778 and 4,704,692) describes methods for preparing single polypeptide chain antibodies. Ward et al., Nature 341:544-546, 1989, describes the preparation of heavy chain variable domain which have high antigen-binding affinities. McCafferty et al. (Nature 348:552-554, 1990) show that complete antibody V domains can be displayed on the surface of fd bacteriophage, that the phage bind specifically to antigen, and that rare phage (one in a million) can be isolated after affinity chromatography. Boss et al. (U.S. Pat. No. 4,816,397) describes various methods for producing immunoglobulins, and immunologically functional fragments thereof, that include at least the variable domains of the heavy and light chains in a single host cell. Cabilly et al. (U.S. Pat. No. 4,816,567) describes methods for preparing chimeric antibodies. In addition, the antibodies can be coupled to compounds, such as toxins or radiolabels.

Prodrugs and Other Modified Compounds

As described above, the MK2 inhibitor may be a small molecule, a peptide, or a nucleic acid molecule. In some instances, a compound that is effective in vitro in inhibiting MK2 polypeptide is not an effective therapeutic agent in vivo. For example, this could be due to low bioavailability of the MK2 inhibitor. One way to circumvent this difficulty is to administer a modified drug, or prodrug, with improved bioavailability that converts naturally to the original compound following administration. Such prodrugs may undergo transformation before exhibiting their full pharmacological effects. Prodrugs contain one or more specialized protective groups that are specifically designed to alter or to eliminate undesirable properties in the parent molecule. In one embodiment, a prodrug masks one or more charged or hydrophobic groups of a parent molecule. Once administered, a prodrug is metabolized in vivo into an active compound.

Prodrugs may be useful for improving one or more of the following characteristics of a drug: solubility, absorption, distribution, metabolization, excretion, site specificity, stability, patient acceptability, reduced toxicity, or problems of formulation. For example, an active compound may have poor oral bioavailability, but by attaching an appropriately-chosen covalent linkage that may be metabolized in the body, oral bioavailability may improve sufficiently to enable the prodrug to be administered orally without adversely affecting the parent compound's activity within the body.

A prodrug may be carrier-linked, meaning that it contains a group such as an ester that can be removed enzymatically. Optimally, the additional chemical group has little or no pharmacologic activity, and the bond connecting this group to the parent compound is labile to allow for efficient in vivo activation. Such a carrier group may be linked directly to the parent compound (bipartate), or it may be bonded via a linker region (tripartate). Common examples of chemical groups attached to parent compounds to form prodrugs include esters, methyl esters, sulfates, sulfonates, phosphates, alcohols, amides, imines, phenyl carbamates, and carbonyls.

As one example, methylprednisolone is a poorly water-soluble corticosteroid drug. In order to be useful for aqueous injection or ophthalmic administration, this drug must be converted into a prodrug of enhanced solubility. Methylprednisolone sodium succinate ester is much more soluble than the parent compound, and it is rapidly and extensively hydrolysed in vivo by cholinesterases to free methylprednisolone.

Caged compounds may also be used as prodrugs. A caged compound may have, e.g., one or more photolyzable chemical groups attached that renders the compound biologically inactive. In this example, flash photolysis releases the caging group (and activates the compound) in a spatially or temporally controlled manner. Caged compounds may be made or designed by any method known to those of skill in the art.

For further description of the design and use of prodrugs, see Testa and Mayer, Hydrolysis in Drug and Prodrug Metabolism: Chemistry, Biochemistry and Enzymology, published by Vch. Verlagsgesellschaft Mbh. (2003).

Other modified compounds are also possible in the methods of the invention. For example, a modified compound need not be metabolized to form a parent molecule. Rather, in some embodiments, a compound may contain a non-removable moiety that, e.g., increases bioavailability without substantially diminishing the activity of the parent molecule. Such a moiety could, for example, be covalently-linked to the parent molecule and could be capable of translocating across a biological membrane such as a cell membrane, in order to enhance cellular uptake. Exemplary moieties include peptides, e.g., penetratin or TAT. An exemplary penetratin-containing compound according to the invention is, e.g., a peptide comprising the sixteen amino acid sequence from the homeodomain of the Antennapedia protein (Derossi et al., J. Biol. Chem. 269:10444-10450, 1994), particularly a peptide having the amino acid sequence RQIKIWFQNRRMKWKK (SEQ ID NO: 26), or including a peptide sequence disclosed by Lin et al. (J. Biol. Chem. 270:14255-14258, 1995). Others are described in U.S. Patent Application Publication No. 2004-0209797 and U.S. Pat. Nos. 5,804,604, 5,747,641, 5,674,980, 5,670,617, and 5,652,122. In addition, a compound of the invention could be attached, for example, to a solid support.

### Chemotherapeutic Agents

A cancer patient identified as having cancer cell(s) with an inactivated MK2 pathway (e.g., cells with an inactivated MK2 pathway and an inactivated p53 pathway) may selectively benefit from the administration of one or more (e.g., two, three, four, or five) chemotherapeutic agent(s) relative to a patient having a cancer cell(s) with an activated MK2 pathway and/or p53 pathway. For example, cancer patients that are implicated as having an inactivated MK2 pathway (e.g., patients with an inactivated MK2 pathway and an inactivated p53 pathway) may experience at least a 10% (e.g., at least 20%, 30%, 40%, 50%, 60%, 70%, 80%, or 90%) in one or more symptoms of cancer following treatment with one or more chemotherapeutic agents compared to a cancer subject having cancer cells with an activated MK2 pathway and an inactivated p53 pathway following treatment with the same chemotherapeutic agents. In view of Applicant's discovery, a skilled physician may recommend to a patient having cancer cells with an inactivated MK2 pathway (e.g., cancer cells with an inactivated MK2 pathway and an inactivated p53 pathway), a therapeutic regime that includes the administration of one or more chemotherapeutic agents (e.g. the administration of an additional dosage of a chemotherapeutic agent to a patient that has previously received a dosage of a chemotherapeutic agent). In addition, a cancer patient diagnosed as having a chemotherapy-sensitive cancer (e.g., by a diagnostic or clinical laboratory) using the diagnostic methods described herein, may be administered one or more chemotherapeutic agent(s).

A variety of chemotherapeutic agents are known in the art. Desirably, the chemotherapeutic agent administered is able to induce genotoxic stress (e.g., DNA-damage). Non-limiting examples of chemotherapeutic agents include: alemtuzumab, altretamine, aminoglutethimide, amsacrine, anastrozole, azacitidine, bleomycin, bicalutamide, busulfan, capecitabine, carboplatin, carmustine, celecoxib, chlorambucil, 2-chlorodeoxyadenosine, cisplatin, colchicine, cyclophosphamide, cytarabine, cytoxan, dacarbazine, dactinomycin, daunorubicin, docetaxel, doxorubicin, epirubicin, estramustine phosphate, etodolac, etoposide, exemestane, floxuridine, fludarabine, 5-fluorouracil, flutamide, formestane, gemcitabine, gentuzumab, goserelin, hexamethylmelamine, hydroxyurea, hypericin, ifosfamide, imatinib, interferon, irinotecan, letrozole, leuporelin, lomustine, mechlorethamine, melphalen, mercaptopurine, 6-mercaptopurine, methotrexate, mitomycin, mitotane, mitoxantrone, nilutamide, nocodazole, paclitaxel, pentostatin, procarbazine, raltitrexed, rituximab, rofecoxib, streptozocin, tamoxifen, temozolomide, teniposide, 6-thioguanine, topotecan, toremofine, trastuzumab, vinblastine, vincristine, vindesine, and vinorelbine.

### Administration Schedules and Formulations

The methods of treatment provided by the invention may require the steps of determining the activation or inactivation of the MK2 signaling pathway and, optionally, steps of determining the inactivation of the p53 signaling pathway. Upon a determination that a cancer patient has an inactivated MK2 pathway (e.g., a cancer patient having an inactivated p53 pathway and an inactivated p53 pathway), the patient is administered one or more dosages (e.g., at least two, three, four, five, six, seven, eight, nine, or ten dosages) of a one or more (e.g., two, three, four, or five) chemotherapeutic agents. Upon a determination that a cancer patient has an activated MK2 pathway (e.g., a cancer patient having an activated MK2 pathway and an inactivated p53 pathway), the patient is administered one or more dosages (e.g., at least two, three, four, five, six, seven, eight, nine, or ten dosages) or one or more (e.g., two, three, four, or five) MK2 inhibitor(s). In certain embodiments of these methods, the determination of inactivation or activation of the MK2 pathway and, optionally the determination of inactivation of the p53 pathway, is performed by a diagnostic or clinical laboratory. Following a determination that a cancer patient has an activated MK2 pathway (e.g., a cancer patient having an activated MK2 pathway and an inactivated p53 pathway), the patient is administered one or more dosages (e.g., at least two, three, four, five, six, seven, eight, nine, or ten dosages) of one or more (e.g., two, three, four, or five) MK2 inhibitors or one or more dosages (e.g., at least two, three, four, five, six, seven, eight, nine, or ten dosages) of one or more (e.g., two, three, four, or five) MK2 inhibitors and one or more (e.g., two, three, four, or five) chemotherapeutic agents.

In any of the methods described herein, each of the one or more MK2 inhibitors may be administered in dosage of 0.1 mg and 1 g, 0.1 mg and 750 mg, 0.1 mg and 600 mg, 0.1 mg and 500 mg, 10 mg and 450 mg, 10 mg and 400 mg, 10 mg and 350 mg, 10 mg and 350 mg, and 10 mg and 250 mg. The specific dosage of each MK2 inhibitor to be administered to the subject may vary depending upon the chemical nature of the MK2 inhibitor. The MK2 inhibitor(s) may be formulated for any known route of administration, including oral, intravenous, intraarterial, intraocular, intranasal, intramuscular, and subcutaneous administration. The MK2 inhibitor may be administered to cancer patients once a day, twice a day, three times a day, once a week, twice a week, three times a week, four times a week, five times a week, six times a week, seven times a week, bi-weekly, tri-weekly, monthly, every two months, every three months, every four months, every five months, twice a year, three times a year, four times a year, five times a year, or six times a year. The specific dosage and administration schedule for a MK2 inhibitor may be determined by a skilled physician based on a number of factors including the age, weight, and sex of the patient, the type of cancer, and the severity of one or more symptoms of cancer.

In any of the methods described herein, each of the one or more chemotherapeutic agents may be administered in a dosage of 0.1 mg and 1 g, 0.1 mg and 750 mg, 0.1 mg and 600 mg, 0.1 mg and 500 mg, 10 mg and 450 mg, 10 mg and 400 mg, 10 mg and 350 mg, 10 mg and 350 mg, and 10 mg and 250 mg. The specific dosage of each chemotherapeutic agent to be administered to the subject may vary depending upon the chemical nature of the chemotherapeutic agent. The chemotherapeutic agent(s) may be formulated for any known route of administration, including oral, intravenous, intraarterial, intraocular, intranasal, intramuscular, and subcutaneous administration. The chemotherapeutic agent may be administered to cancer patients once a day, twice a day, three times a day, once a week, twice a week, three times a week, four times a week, five times a week, six times a week, seven times a week, bi-weekly, tri-weekly, monthly, every two months, every three months, every four months, every five months, twice a year, three times a year, four times a year, five times a year, or six times a year. The specific dosage and administration schedule for a chemotherapeutic agent may be determined by a skilled physician based on a number of factors including the age, weight, and sex of the patient, the type of cancer, and the severity of one or more symptoms of cancer.

In instances where a cancer patient is administered the combination of one or more (e.g., two, three, four, five, or six) MK2 inhibitors and one or more (e.g., two, three, four, five, or six) chemotherapeutic agents, the one or more MK2 inhibitors and the one or more chemotherapeutic agents may be administered at the same time (e.g., administered in the same formulated dose). In another example, the one or more MK2 inhibitors may be administered to the cancer patient prior to the administration of the one or more chemotherapeutic agents (e.g., wherein the bioactive period of the one or more MK2 inhibitors overlaps with the bioactive period of the one or more chemotherapeutic agents).

In a further example, the one or more chemotherapeutic agents may be administered to the cancer patient prior to the administration of the one or more MK2 inhibitors (e.g., wherein the bioactive period of the one or more MK2 inhibitors overlaps with the bioactive period of the one or more chemotherapeutic agents). The therapeutic methods provided by the invention may be performed alone or in conjunction with another cancer therapy and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the patient's cancer, and how the patient responds to the treatment. Additionally, a person having a greater risk of developing cancer may be treated by the methods of the invention (e.g., a person who is genetically predisposed). Therapy, as provided by the invention, may be given in on-and-off cycles that include rest periods so that the patient's body has a chance to build healthy new cells and regain its strength. Therapy may be used to extend the patient's lifespan.

For cancer treatment, depending on the type of cancer and its stage of development, the therapy can be used to slow the spreading of the cancer, to slow the cancer's growth, to kill or arrest cancer cells that may have spread to other parts of the body from the original tumor, or to relieve symptoms caused by the cancer.

### Combination Therapies

In addition to the MK2 inhibitors, chemotherapeutic agents, or the combination of MK2 inhibitors and chemotherapeutic agents described above, the cancer patient may also be treated with one or more (e.g., two, three, four, or five) additional agents including one or more (e.g., one, two, three, four, or five) non-steroidal anti-inflammatory drug(s) (NSAID(s)), one or more (e.g., two, three, four, or five) immunosuppressive agent(s), one or more (e.g., two, three, four, or five) calcineurin inhibitor(s), or one or more (e.g., two, three, four, or five) analgesic(s). Examples of NSAIDs, immunosuppressive agents, and analgesics are known in the art.

Depending on the type of cancer and its stage of development, the combination therapy can be used to treat cancer, to slow the spreading of the cancer, to slow the cancer's growth, to kill or arrest cancer cells that may have spread to other parts of the body from the original tumor, to relieve symptoms caused by the cancer, or to prevent cancer in the first place. Combination therapy can also help people live more comfortably by eliminating cancer cells that cause pain or discomfort.

The administration of any of the above combinations of agents (e.g., combination of MK2 inhibitors and chemotherapeutic agents) of the present invention allows for the administration of lower doses of each compound, providing similar efficacy and lower toxicity compared to administration of either compound alone. Alternatively, such combinations result in improved efficacy in treating cancer with similar or reduced toxicity.

### Treatment Evaluation

The methods provided by the invention may be used to treat an individual having any type of cancer (e.g., an individual diagnosed as having a cancer). Non-limiting examples of cancer that may be treated by the provided methods include: acoustic neuroma, acute leukemia, acute lymphocytic leukemia, acute monocytic leukemia, acute myeloblastic leukemia, acute myelocytic leukemia, acute myelomonocytic leukemia, acute promyelocytic leukemia, acute erythroleukemia, adenocarcinoma, angiosarcoma, astrocytoma, basal cell carcinoma, bile duct carcinoma, bladder carcinoma, brain cancer, breast cancer, bronchogenic carcinoma, cervical cancer, chondrosarcoma, chordoma, choriocarcinoma, chronic leukemia, chronic lymphocytic leukemia, chronic myelocytic leukemia, colon cancer, colon carcinoma, craniopharyngioma, cystadenocarcinoma, embryonal carcinoma, endotheliosarcoma, ependymoma, epithelial carcinoma, Ewing's tumor, glioma, heavy chain disease, hemangioblastoma, hepatoma, Hodgkin's disease, large cell carcinoma, leiomyosarcoma, liposarcoma, lung cancer, lung carcinoma, lymphangioendotheliosarcoma, lymphangiosarcoma, macroglobulinemia, medullary carcinoma, medulloblastoma, melanoma, meningioma, mesothelioma, myxosarcoma, neuroblastoma, non-Hodgkin's disease, oligodendroglioma, osteogenic sarcoma, ovarian cancer, pancreatic cancer, papillary adenocarcinomas, papillary carcinoma, pinealoma, polycythemia vera, prostate cancer, rhabdomyosarcoma, renal cell carcinoma, retinoblastoma, schwannoma, sebaceous gland carcinoma, seminoma, small cell lung carcinoma, squamous cell carcinoma, sweat gland carcinoma, synovioma, testicular cancer, uterine cancer, Waldenstrom's fibrosarcoma, and Wilm's tumor.

A skilled physician may monitor the effectiveness of treatment of a cancer by monitoring the severity or duration of one or more symptoms of cancer. Non-limiting examples of symptoms of cancer include: blood in urine, pain or burning upon urination, cloudy urine, pain in bone, fractures in bones, fatigue, weight loss, repeated infections, nausea, vomiting, constipation, numbness in the legs, bruising, dizziness, drowsiness, abnormal eye movements, changes in vision, changes in speech, headaches, thickening of a tissue, rectal bleeding, abdominal cramps, loss of appetite, fever, enlarged lymphnodes, persistent cough, blood in sputum, lung congestion, itchy skin, lumps in skin, abdominal swelling, vaginal bleeding, jaundice, heartburn, indigestion, cell proliferation, and loss of regulation of controlled cell death.

The methods of treatment provided by the invention may result in at least a 5% (e.g., at least 10%, 15%, 20%, 25%, 30%, 35%, 40%, 45%, 50%, 55%, 60%, 65%, 70%, 75%, 80%, 85%, 90%, or 95% decrease) in one or more symptoms (e.g., two, three, four, or five symptoms) of cancer (e.g., those symptoms listed above). The methods of treatment may also provide a decrease in the toxicity normally observed for a MK2 inhibitor and/or a chemotherapeutic agent. The methods of treatment may also provide for a reduction in the dosage of a MK2 inhibitor or a chemotherapeutic agent necessary to achieve a therapeutic effect (e.g., a reduction in one or more symptoms of cancer). Desirably, the provided methods may result in a decrease in the metastasis or recurrence of cancer in a patient or may provide for an increase in the duration of remission in a patient.

### Methods for Identifying Patient Populations for Selective Therapeutic Treatment

As indicated herein, patients with cancer cell(s) that have an inactivated MK2 signaling pathway (e.g., patients with an inactivated MK2 signaling pathway and an inactive p53 signaling pathway) have a decreased G₂/S checkpoint function, and therefore, are more sensitive to chemotherapeutic agents (e.g., DNA damaging agents). For example, patients having an inactivated MK2 signaling pathway and an inactivated p53 signaling pathway have decreased G₁ and G₂/S checkpoint function are more sensitive to chemotherapeutic agents. Thus, a physician, in determining the treatment regime for a cancer patient, may suggest the administration of one or more chemotherapeutic agent(s) (e.g., an additional dosage of a chemotherapeutic agent) to a patient having cancer cell(s) with an inactivated MK2 signaling pathway (e.g., a patient having cancer cell(s) with an inactivated MK2 signaling pathway and an inactivated p53 signaling pathway). Similarly, a patient having cancer cells(s) that have an activated MK2 pathway (e.g., a patient having cancer cells with an activated MK2 pathway and an inactivated p53 pathway) may selectively benefit from the administration of one or more MK2 inhibitor(s) or a combination of one or more MK2 inhibitor(s) and one or more chemotherapeutic agent(s) to the G₂/S checkpoint function in the cancer cells (or decrease both G₁ and G₂/S checkpoint function in cancer cells having an inactivated MK2 signaling pathway and an inactivated p53 pathway). Thus, the invention provides methods that allow a physician to identify a specific subset of patients that may selectively benefit from the administration of one or more chemotherapeutic agent(s) (e.g., cancer patients having cancer cell(s) with inactivated MK2 pathway and, optionally an inactivated p53 pathway) or administration of one or more MK2 inhibitor(s) or the combination of one or more MK2 inhibitor(s) and one or more chemotherapeutic agent(s) (e.g., cancer patients having cancer cell(s) with activated MK2 pathway and, optionally, an inactivated p53 pathway). These methods require steps for the determination of the activation or inactivation of the MK2 pathway and, optionally, steps for the determination of the inactivation of the p53 pathway (as described above). As noted above, these methods allow a physician to identify a patient that may selectively benefit from the administration of a MK2 inhibitor, a chemotherapeutic agent, or a combination of a MK2 inhibitor and a chemotherapeutic agent (e.g., the identified patient would experience at least a 10% decrease in one or more symptoms of cancer relative to another cancer patient receiving the same treatment (e.g., a cancer patient having cells with activated MK2 pathway and inactivated p53 pathway, a cancer patient having cells with activated MK2 pathway and activated p53 pathway)).

### Methods for Diagnosing a Chemotherapy-Sensitive or a Chemotherapy-Resistant Cancer

The invention further provides method for diagnosing a chemotherapy-sensitive or a chemotherapy-resistant cancer in a patient. As described above, cancer cells having an inactivated MK2 pathway (e.g., cancer cells having an inactivated MK2 pathway and an inactivated p53 pathway) are more sensitive to treatment with one or more chemotherapeutic agent(s) (e.g., an agent that induces genotoxic stress, such as an agent that induces DNA damage) compared to non-cancer cells or other cancer cells (e.g., cells having an activated MK2 pathway and an inactive p53 pathway, an activated MK2 pathway and an activated p53 pathway, or an inactivated MK2 pathway and an active p53 pathway). In addition, cancer cells having an activated MK2 pathway (e.g., cancer cells having an activated MK2 signaling pathway and an inactivated p53 pathway) are more sensitive to treatment with one or more MK2 inhibitor(s) or a combination of one or more MK2 inhibitor(s) and one or more chemotherapeutic agent(s) (e.g., an agent that induces genotoxic stress, e.g., an agent that induces DNA damage) compared to non-cancer cells or other cancer cells having an active MK2 pathway (e.g., cancer cells having an active MK2 pathway and an inactive p53 pathway or cancer cells having an active MK2 pathway and an active p53 pathway). Thus these methods allow for the diagnosis of a chemotherapy-sensitive cancer in patient by measuring the inactivation of the MK2 pathway and, optionally, measuring the inactivation of the p53 pathway in a cancer cell from the patient, wherein a patient having cancer cell(s) with inactivated MK2 pathway (e.g., cancer cells with inactivated MK2 pathway and inactivated p53 pathway) are diagnosed as having a chemotherapy-sensitive cancer (e.g., indicating that these patients have cancer that may be sensitive to treatment that includes the administration of one or more chemotherapeutic agents). The invention also provides methods for diagnosing of a chemotherapy-resistant cancer in a patient by measuring the activation of the MK2 pathway and, optionally, measuring the inactivation of the p53 pathway in a cancer cell from the patient, wherein a patient having cancer cell(s) with activated MK2 pathway (e.g., cancer cells with an activated MK2 pathway and an inactivated p53 pathway) are diagnosed as having a chemotherapy-resistant cancer (e.g., indicating that these patients have cancer that may be sensitive to treatment that includes the administration of one or more MK2 inhibitors).

These methods require steps for the determination of the activation or inactivation of the MK2 pathway and, optionally, the inactivation of the p53 pathway (as described above). As noted above, these methods allow a physician to diagnose a patient that has a chemotherapy-resistant cancer (e.g., a cancer that may be sensitive to treatment with one or more MK2 inhibitors) or a chemotherapy-sensitive cancer (e.g., a cancer that may be sensitive to treatment with one or more chemotherapeutic agents). Such diagnosis of cancer in a patient may allow the physician to select a specific therapeutic regime for the cancer patient.

### Kits

The invention further provides kits that provide reagents for diagnosing a chemotherapy-resistant cancer or a chemotherapy-sensitive cancer in a subject. Such kits may contain for example one or more reagent(s) (e.g., two, three, four, five, or six reagents) capable of measuring one or more feature(s) (e.g., two, three, four, five, or six features) in a cancer cell(s) from a patient selected from the group of: cytoplasmic or nuclear MK2 protein localization, phosphorylation of total MK2 protein, levels of phosphorylated MK2 protein in the cytoplasm or nucleus, levels of phosphorylated hsp27, levels of phosphorylated hnRNPA0, and levels of phosphorylated PARN, levels of phosphorylated TIAR, levels of phosphorylated cdc25B, levels of cdc25C, and levels of Gadd45a protein or mRNA; and one or more reagents (e.g., two, three, four, five, or six reagents) capable of capable of measuring one or more feature(s) (e.g., two, three, four, five, or six features) in a cancer cell(s) from said patient selected from the group consisting of: tumor protein-53 (p53) mRNA or protein levels, expression of a mutant or truncated p53 with decreased expression or activity, and p21 expression or activity. The kits may further include instructions for using the above reagents to determine the presence of a chemotherapy-resistant or chemotherapy-sensitive cancer in the patient.

Non-limiting examples of reagents that may be provided in the kits include: antibodies that bind to phosphorylated, nonphosphorylated, or total MK2 protein; antibodies that bind to phosphorylated, nonphosphorylated, or total hsp27; antibodies that bind to phosphorylated, nonphosphorylated, or total hnRNPA0; antibodies that bind to phosphorylated, nonphosphorylated, or total PARN; antibodies that bind to Gadd45a protein; an oligonucleotide containing a sequence complementary to a nucleic acid sequence encoding Gadd45a protein; antibodies that bind to phosphorylated, nonphosphorylated, or total TIAR; antibodies that bind to phosphorylated, nonphosphorylated, or total cdc25B; antibodies that hind to phosphorylated, nonphosphorylated, or total cdc25C; nucleic acid primers that may be used to amplify a Gadd45a mRNA; antibodies that bind to p53; an oligonucleotide containing a sequence complementary to a nucleic acid sequence encoding p53 (e.g., encoding wild type p53 protein or a mutant or truncated p53 protein); nucleic acid primers that may be used to amplify a p53 mRNA or gene (e.g., a mRNA or gene encoding wild type p53 protein or a mRNA or gene encoding mutant or truncated p53 protein); antibodies that bind to p21; an oligonucleotide containing a sequence complementary to a nucleic acid sequence encoding p21; nucleic acid primers that may be used to amplify a p21 mRNA; and labeled peptide substrates for activated MK2 protein (e.g., peptide substrates having the consensus sequence of [L/F/I]XR[Q/S/T]L[S/T][hydrophobic] (SEQ ID NO: 5) and having a total of less than 50 amino acids). Examples of the above-referenced antibodies are commercially available or may be purified using techniques known in the art (described above for MK2 antibodies). For example, primers and antisense oligonucleotides for measuring Gadd45a, p53, and p21 expression (mRNA or gene expression) may be designed a skilled artisan based on the sequences described herein and those sequences known in art.

The instructions provided with the kit may describe that the use of one or more of the above reagents to measure one or more (e.g., two, three, four, or five) features of MK2 pathway activation or one or more (e.g., two, three, four, or five) features of MK2 pathway inactivation, and, optionally, the use of one of more of the above reagents to measure one or more (e.g., two, three, four, or five) features of p53 pathway inactivation. For example, non-limiting molecular biology protocols to measure MK2 pathway inactivation, MK2 pathway activation, and p53 pathway inactivation are described above.

Using the reagents provided in the kits, MK2 pathway activation may be indicated by the observance of one or more (e.g., two, three, four, five, or six) of following features: increased (e.g., by at least 10%, 20%, 30%, 40%, 50%, 60%, 70%, 80%, or 90%) MK2 protein in the cytoplasm, decreased (e.g., by at least 10%, 20%, 30%, 40%, 50%, 60%, 70%, 80%, or 90%) MK2 in the nucleus, increased (e.g., by at least 10%, 20%, 30%, 40%, 50%, 60%, 70%, 80%, or 90%) total MK2 protein phosphorylation, increased (e.g., by at least 10%, 20%, 30%, 40%, 50%, 60%, 70%, 80%, or 90%) levels of phosphorylated MK2 in the cytoplasm or nucleus, increased (by at least 10%, 20%, 30%, 40%, 50%, 60%, 70%, 80%, or 90%) levels of phosphorylated cdc25B (e.g., phosphorylation at serine 323), increased (by at least 10%, 20%, 30%, 40%, 50%, 60%, 70%, 80%, or 90%) levels of phosphorylated cdc25C (e.g., phosphorylation at serine 216), increased (by at least 10%, 20%, 30%, 40%, 50%, 60%, 70%, 80%, or 90%) levels of phosphorylated TIAR, increased (by at least 10%, 20%, 30%, 40%, 50%, 60%, 70%, 80%, or 90%) levels of phosphorylated hnRNPA0 (e.g., phosphorylation at serine 84), increased (by at least 10%, 20%, 30%, 40%, 50%, 60%, 70%, 80%, or 90%) PARN phosphorylation (e.g., phosphorylation at serine 557), and increased (by at least 10%, 20%, 30%, 40%, 50%, 60%, 70%, 80%, or 90%) levels of phosphorylated hsp-27 (e.g., phosphorylation at serine 15, serine 78, and/or serine 82).

Conversely, inactivated MK2 signaling pathway may be indicated by the observance of one or more (e.g., two, three, four, five, or six) of the following features: decreased (by at least 10%, 20%, 30%, 40%, 50%, 60%, 70%, 80%, or 90%) MK2 protein in the cytoplasm, increased (by at least 10%, 20%, 30%, 40%, 50%, 60%, 70%, 80%, or 90%) MK2 in the nucleus, decreased (by at least 10%, 20%, 30%, 40%, 50%, 60%, 70%, 80%, or 90%) total MK2 protein phosphorylation, decreased (by at least 10%, 20%, 30%, 40%, 50%, 60%, 70%, 80%, or 90%) levels of phosphorylated MK2 in the cytoplasm or nucleus, decreased (by at least 10%, 20%, 30%, 40%, 50%, 60%, 70%, 80%, or 90%) levels of phosphorylated cdc25B (e.g., phosphorylation at serine 323), decreased (by at least 10%, 20%, 30%, 40%, 50%, 60%, 70%, 80%, or 90%) levels of phosphorylated cdc25C (e.g., phosphorylation at serine 216), decreased (by at least 10%, 20%, 30%, 40%, 50%, 60%, 70%, 80%, or 90%) levels of phosphorylated TIAR, decreased (by at least 10%, 20%, 30%, 40%, 50%, 60%, 70%, 80%, or 90%) levels of phosphorylated hnRNPA0 (e.g., phosphorylation at serine 84), decreased (by at least 10%, 20%, 30%, 40%, 50%, 60%, 70%, 80%, or 90%) levels of phosphorylated PARN (e.g., phosphorylation at serine 557), and decreased (by at least 10%, 20%, 30%, 40%, 50%, 60%, 70%, 80%, or 90%) levels of phosphorylated hsp-27 (e.g., phosphorylation at serine 15, serine 78, and/or serine 82).

p53 pathway inactivation is indicated by the observance of one or more (e.g., two, three, four, five, or six) of the following features: decreased (e.g., by at least 10%, 20%, 30%, 40%, 50%, 60%, 70%, 80%, or 90%) p53 mRNA or protein levels, expression of a mutant or truncated p53 with decreased (e.g., by at least 10%, 20%, 30%, 40%, 50%, 60%, 70%, 80%, or 90%) expression or activity, and decreased (by at least 10%, 20%, 30%, 40%, 50%, 60%, 70%, 80%, or 90%) p21 expression or activity.

The above features of MK2 pathway inactivation or MK2 pathway activation and p53 pathway inactivation may be performed using a sample of cells from a patient (e.g., a biopsy sample or blood sample) or a cellular lysate prepared from cells from a patient. A patient that is measured as having cells with one or more features of MK2 pathway activation and, optionally, one or more features of p53 pathway inactivation, is diagnosed as having a chemotherapy-resistant cancer (e.g., a patient that may benefit from the administration of one or more MK2 inhibitor(s) or the combination of one or more MK2 inhibitor(s) and one or more chemotherapeutic agent(s)). A patient that is measured as having cells with one or more features of MK2 pathway inactivation and, optionally, one or more features of p53 pathway inactivation, is diagnosed as having a chemotherapy-sensitive cancer (e.g., a patient that may benefit from administration of one or more chemotherapeutic agent(s)).

The features and other details of the invention will now be more particularly described and pointed out in the following examples describing preferred techniques and experimental results. These examples are provided for the purpose of illustrating the invention and should not be construed as limiting.

### EXAMPLES

### Example 1

### Chk1 and MK2 Control Early and Late G₂/M Checkpoints, Respectively, after DNA Damage

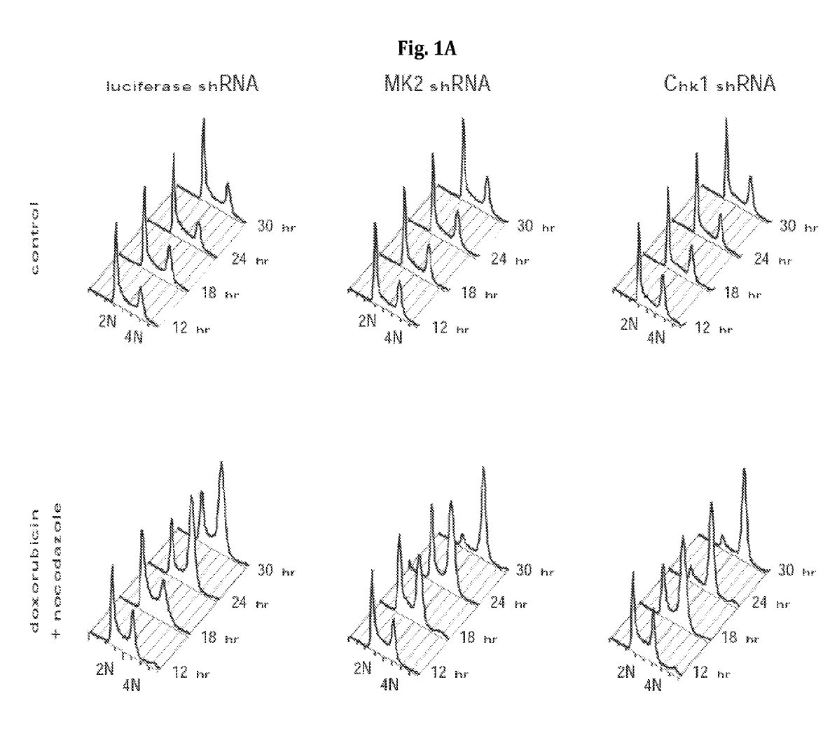

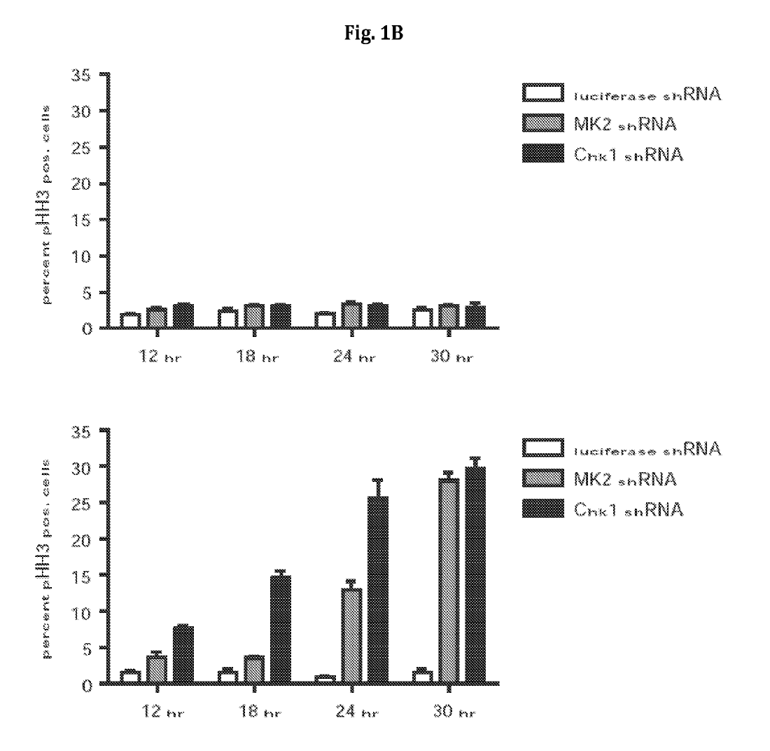

Experiments were performed to determine the role of Chk1 and MK2 in the G₂/M checkpoint in p53-deficient cells following DNA damage. In a first set of experiments, U2OS cells were infected with lentiviruses delivering luciferase-, MK2-, or Chk1-specific shRNAs. The ability of ability of these cells to engage and maintain functional cell-cycle checkpoint following genotoxic stress (1 hour of 1 mM doxorubicin) was analyzed using a FACS-based nocodazole trap experiment. The data from these experiments are shown in FIG. 1A and are described below.

As shown in the upper panels of FIG. 1A, knockdown of MK2 or Chk1 did not result in gross cell-cycle changes in the absence of DNA damage. Treatment of control cells with doxorubicin resulted in a gradual build-up of G₂-arrested cells over 24 hours, as evidenced by the accumulation of 4N cells staining negatively for pHH3 (FIG. 1A, lower panels, and FIG. 1B). Chk1-depleted cells, like wild-type cells, displayed a prominent 4N peak after DNA damage; however, by as early as 12 hours after doxorubin, 7.7% of the cells already stained positively for pHH3. This percentage of pHH3-positive cells progressively rose to 15% by 18 hours and reached 25.6% and 29.5% by 24 and 30 hours, respectively, compared to 0.9% and 1.5% of the shRNA controls at these times. These latter pHH3 values for the Chk1 knockdown cells are similar to those seen in undamaged wild type cells arrested in mitosis with nocodazole, and indicate an early failure of the G2 checkpoint in Chk-1 depleted cells.

In contrast to Chk1 depletion, examination of the MK2 knockdown cells showed an accumulation of 4N DNA-containing cells that were largely negative for pHH3 staining at 12 and 18 hours after doxorubin (3.7%±1.2% and 3.6±0.5%, respectively), indicating a functional early G₂ arrest. However, by 24 and 30 hours following doxorubicin, a gradual collapse of the checkpoint was evident with 13.2% and 28.5% of MK2-depleted cells now staining positively for pHH3 (FIG. 1B). Thus, MK2 depletion appears to disrupt maintenance of the G₂ checkpoint at late times, whereas Chk1 depletion resulted in impaired checkpoint initiation and/or maintenance at earlier times. Expression of shRNA-resistant wild-type MK2 completely rescued the effect of MK2 depletion on doxorubicin-induced G₂/M arrest, whereas expression of a kinase-dead MK2 mutant failed to restore these checkpoints, although this mutant bound to p38 (data not shown), confirming that MK2 activity itself was required for the late cell-specific checkpoint arrest.

### Example 2

### Distinct Nuclear and Cytoplasmic Locations of Active Chk1 and MK2 Following DNA Damage Mediate Early and Late Checkpoint Functions

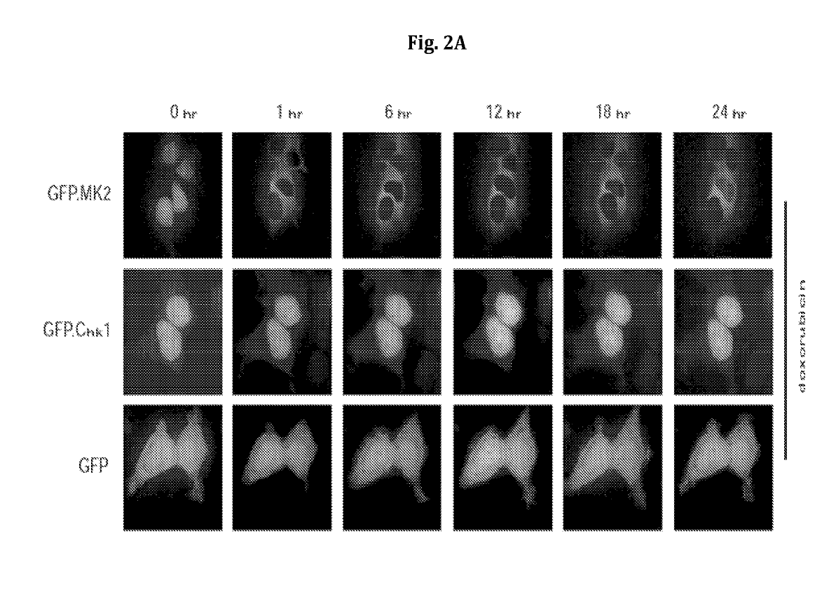

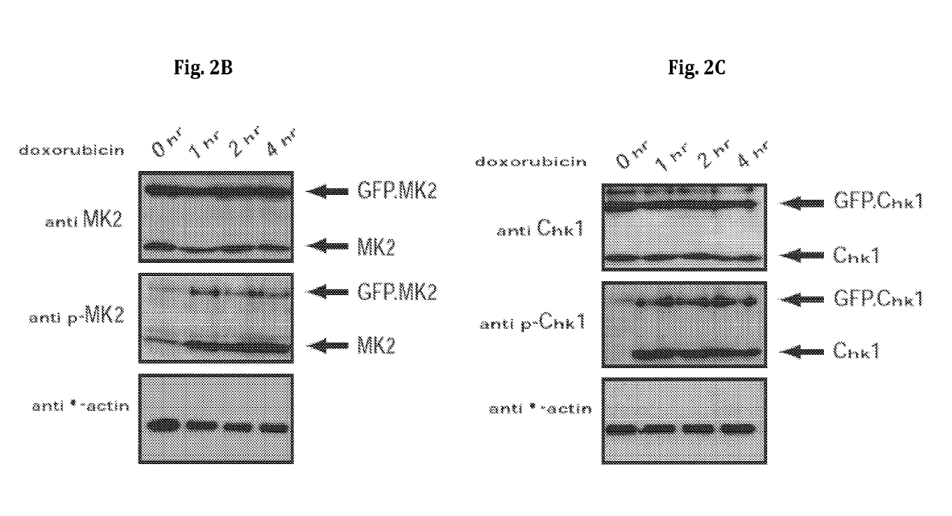

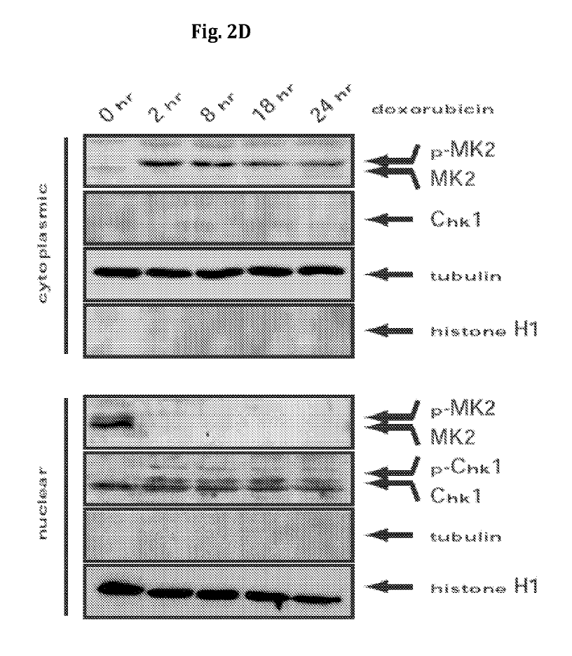

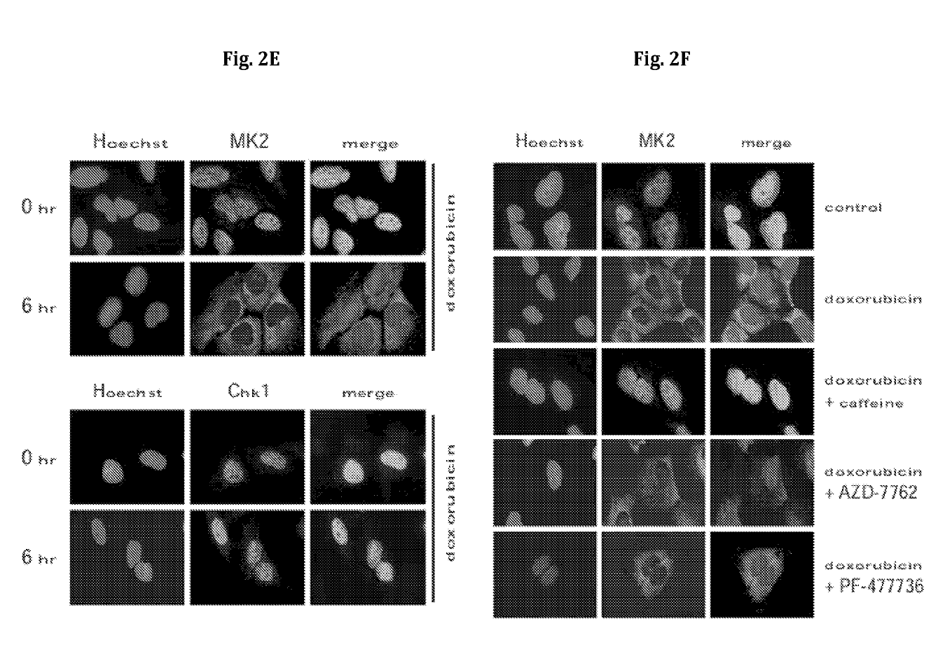

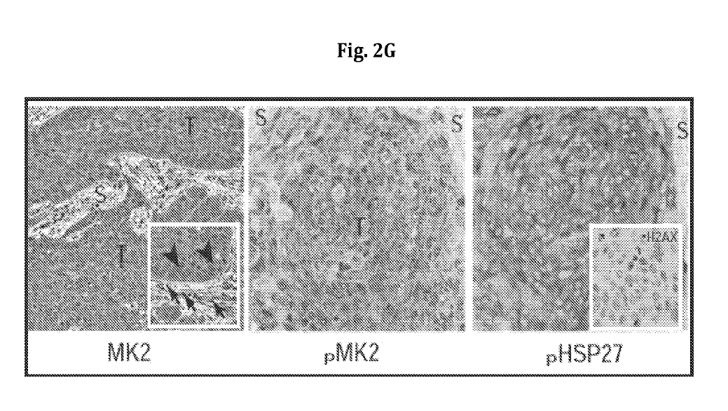

To investigate whether the different temporal kinetics of check point escape seen in the Chk1- and MK2-deficient cells resulted from targeting spatially distinct substrate pools, the subcellular localization of these two checkpoint kinases after genotoxic stress was examined. In these experiments, retroviral gene delivery was used to obtain stable low-level expression of GFP chimeras of Chk1 and MK2 in U2OS cells, and localization was monitored in live cells before and after DNA damage over time. Both GFP-Chk1 and GFP-MK2 localized exclusively in the nucleus of resting (untreated) cells, while GFP alone was diffusely distributed throughout both the cytoplasm and the nucleus (FIG. 2A). Following doxorubicin treatment, GFP-MK2 rapidly translocated from the nucleus to the cytoplasm, where it remained for at least 24 hours, whereas GFP-Chk1 remained nuclear. Phosphorylation/activation of the GFP fusion proteins following DNA damage occurred with identical kinetics as those seen for the endogenous Chk1 and MK2 (FIGS. 2B and 2C), with the damage-induced relocalization of MK2 completely dependent upon its activation by p38, since cytoplasmic translocation after doxorubicin was completely abolished by the addition of the p38 inhibitor SB203580 (data not shown). Similar results were also shown following cisplatin treatment (data now shown).

To ensure that the visual behavior of GFP fusion proteins assayed in vivo reflected the subcellular localizations of endogenous Chk1 and MK2 kinases following DNA damage, a similar series of biochemical experiments were performed where the localization of endogenous activated Chk1 and MK2 was examined in cell lysates by nuclear and cytoplasmic fractionation. The resulting data show that endogenous phospho-MK2 became detectable in the cytoplasmic fraction shortly after DNA damage, while endogenous phospho-Chk1 remained in the nuclear fraction (FIG. 2D). In addition, indirect immunofluoresence was used to directly monitor the subcellular localization of endogenous Chk1 and MK2 in situ (FIG. 2E). These data confirm that doxorubin induces robust cytoplasmic accumulation of MK2, while Chk1 remains exclusively nuclear. The DNA damage-induced cytoplasmic relocalization of MK2 could be completely inhibited by caffeine (FIG. 2F), indicating that the upstream kinases ATM and ATR mediate activation upon genotoxic stress. Pharmacological inhibition of Chk1 using two different inhibitors, however, failed to prevent the doxorubicin-induced cytoplasmic localization of MK2, indicating that Chk1 and MK2 operate in separate parallel pathways (FIG. 2F). The activation and translocation of MK2, as well as phosphorylation of its cytoplasmic substrate, hsp27, was also observed in some human tumor samples which also displayed hallmarks of ongoing DNA damage (positive nuclear gH2AX staining), likely as a consequence of oncogenic stress (FIG. 2G).

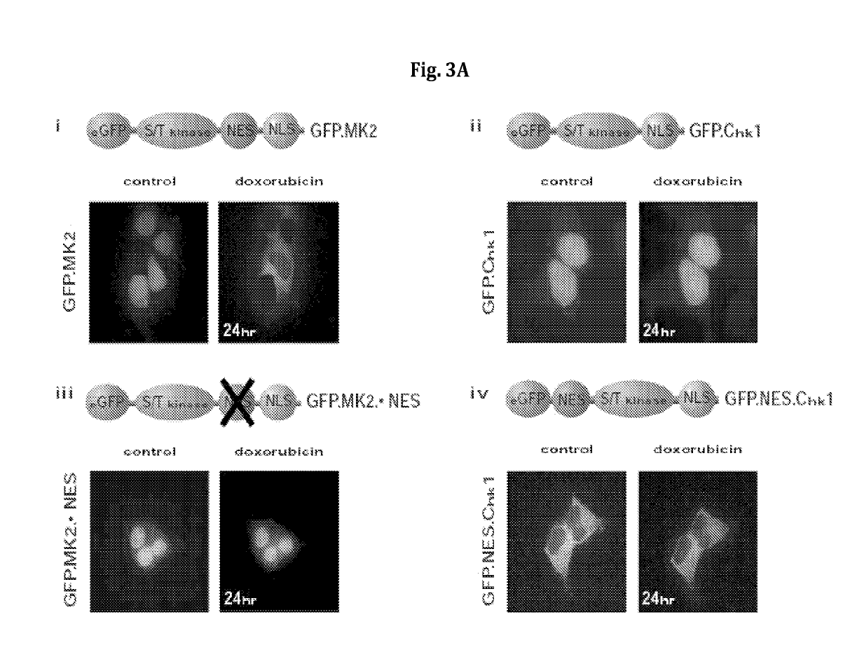

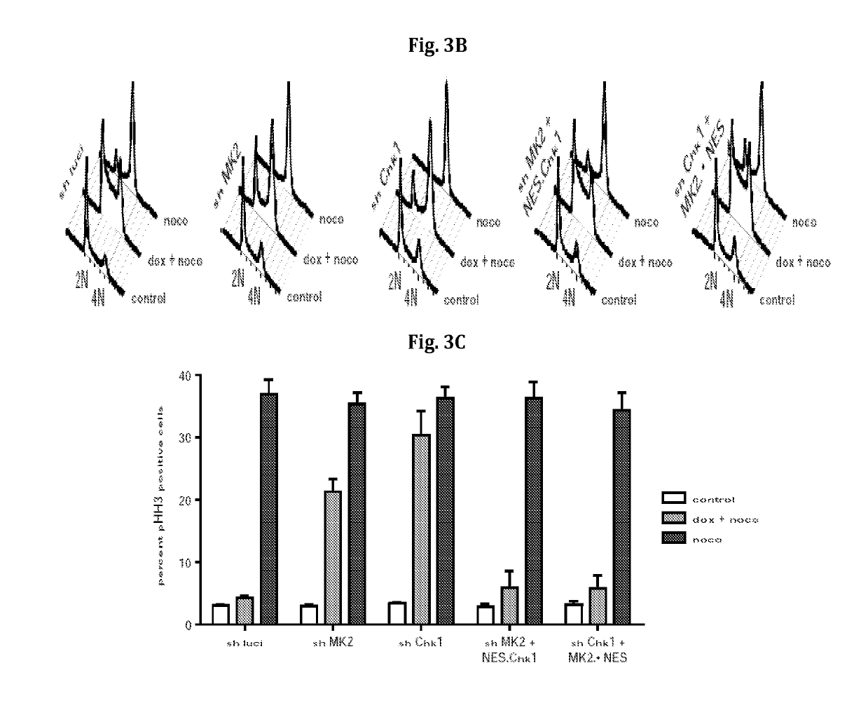

To determine whether the distinct subcellular localization of Chk1 and MK2 after DNA damage were directly responsible for early and late checkpoint arrest, chimeric molecules in which each kinase was spatially substituted for the other within cells were generated. MK2 contains both a bipartite nuclear localization signal (NLS; amino acids 373-389) and a nuclear export signal (NES; amino acids 356-365) located near the C terminus (FIG. 3Ai). In the kinase resting state, the NES is masked by a direct interaction with a hydrophobic patch in the kinase domain. Upon activation and MK2 phosphorylation on Thr-344 by p38, this interaction between the NES and the catalytic core is weakened and the NES becomes exposed, leading to cytoplasmic translocation. In contrast, Chk1 contains a NLS, but lacks a discernable NES (FIG. 3A ii). In order to produce an activatable, but nuclear-restricted form of MK2, we expressed a construct in which the NES was functionally inactivated by insertion of point mutations (FIG. 3A iii). Similarly, to produce cytoplasmic forms of Chk1, constructs in which either the NLS was inactivated or the NES motif from MK2 was inserted at the Chk1 N terminus were generated (FIG. 3A iv). All constructs were fused to GFP to allow visualization of subcellular localization.

The above constructs were expressed in asynchronously growing Chk1- or MK2-knockdown cells, which were left untreated or exposed to doxorubicin for 1 hour, followed by addition of nocodazole for 2 hours after removal of doxorubicin, to capture cells escaping from DNA damage checkpoints in mitosis (FIG. 3B). As an additional control, cells were treated with nocodazole alone. The cells were harvested 30 hours after doxorubicin and cell-cycle disruption was assessed using FACS. Treatment of cells expressing a control shRNA resulted in a robust S and G₂/M checkpoints 30 hours after addition of doxorubicin, with G₂-arrested cells indicated by an accumulation of cells with 4N DNA content that were largely negative for pHH3 staining (FIG. 3C). This arrest was completely abrogated in cells expressing a Chk1 shRNA (FIGS. 3B and 3C). However, MK2.DNES complementation of Chk1-depleted cells resulted in full restoration of functional S and G₂/M checkpoints, indicating that nuclear-targeted MK2 can functionally compensate for the loss of Chk1.

A further set of experiments were performed in order to determine whether forced expression of Chk1 in the cytoplasm could rescue checkpoint defects following loss of MK2. These data again show that knockdown of MK2 in U2OS cells abolished the doxorubicin-induced S and G₂/M cell cycle checkpoints, as evidenced by an accumulation of 21.3% mitotic cells with 4N DNA content staining positive for pHH3 that had escaped cell-cycle checkpoints during the 30-hour course of the experiment (FIGS. 3B and 3C). This value is similar to that of U2OS cells expressing a control shRNA that were blocked in mitosis with nocodazole, in the absence of DNA damage, indicating a complete loss of checkpoint function in MK2-depleted UNOS cells upon doxorubicin treatment. A cytoplasmic Chk1 construct was initially generated by inactivating the Chk1 NLS through mutation of Arg-260/261/270/271 to alanine, resulting in a predominantly cytoplasmic accumulation of Chk1 (Chk1.DNLS) (data not shown). The expression of this construct failed to rescue the checkpoint defects seen in MK2 knockdown cells. Addition of leptomycin B, an inhibitor of Crm-1 dependent nuclear export 12 hours prior to doxorubicin, however, did not result in nuclear entrapment of the Chk1.DNLS protein, indicating that this mutant could not shuttle between the cytoplasm and the nucleus, and was therefore unlikely to be activated by ATR following DNA damage (data not shown). In contrast, leptomycin B treatment resulted in complete entrapment of MK2 within the nucleus despite doxorubicin-induced DNA damage (data not shown). These data demonstrate that nuclear export of MK2 in response to DNA damage is Crm1-dependent.

A second construct was created in which the MK2 NES was placed between GFP and the Chk1 cDNA. This NES.Chk1 construct, like Chk1.DLNS, also showed a predominantly cytoplasmic accumulation of Chk1 (FIG. 3A). Importantly, however, this fusion protein was retained in the nucleus upon leptomycin B treatment, indicating that the NES.Chk1 fusion protein shuttles between cytoplasm and nucleus, where it can be directly activated upon DNA damage (data not shown). Expression of this ATR-activatable, nucleocytoplasmic shuffling form of Chk1 completely rescued the checkpoint defects seen in U2OS cells lacking MK2 (FIGS. 3B and 3C). Notably, when this same Chk1.NES construct was mutated at Ser-317 and Ser-345 to prevent DNA damage-induced phosphorylation, it was unable to reverse the MK2 depletion phenotype, despite its cytoplasmic localization and nuclear-cytoplasmic shuttling. These data indicate that an activated cytoplasmic form of Chk1 can functionally compensate for loss of MK2 activity.

The above data indicate that Chk1 and MK2 control early and late DNA damage checkpoints, respectively, likely through phosphorylating distinct, spatially separated substrate pools following their activation by genotoxic stress.

### Example 3

### MK2 and p38MAPK Activity Results in Long Term Stabilization of Gadd45a Through Phosphorylation of Proteins Involved in RNA Binding and Degradation

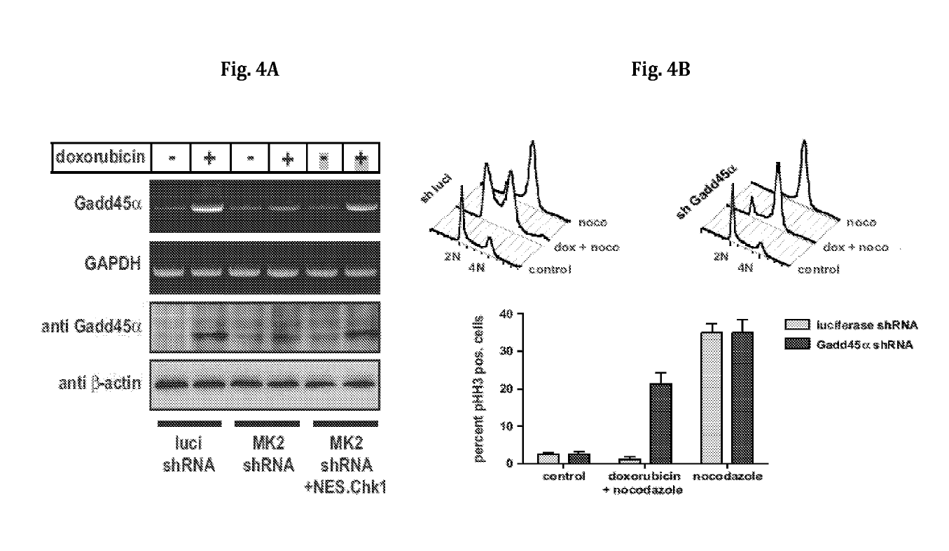

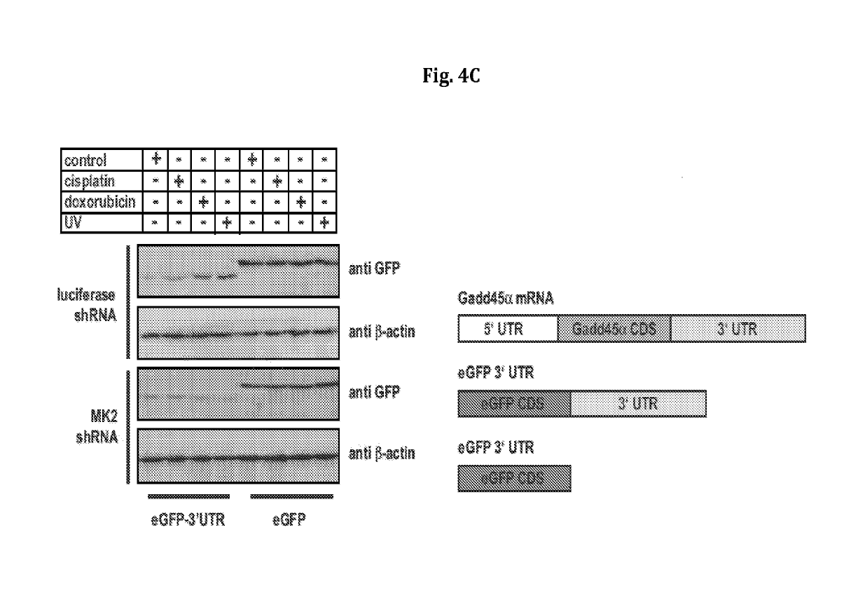

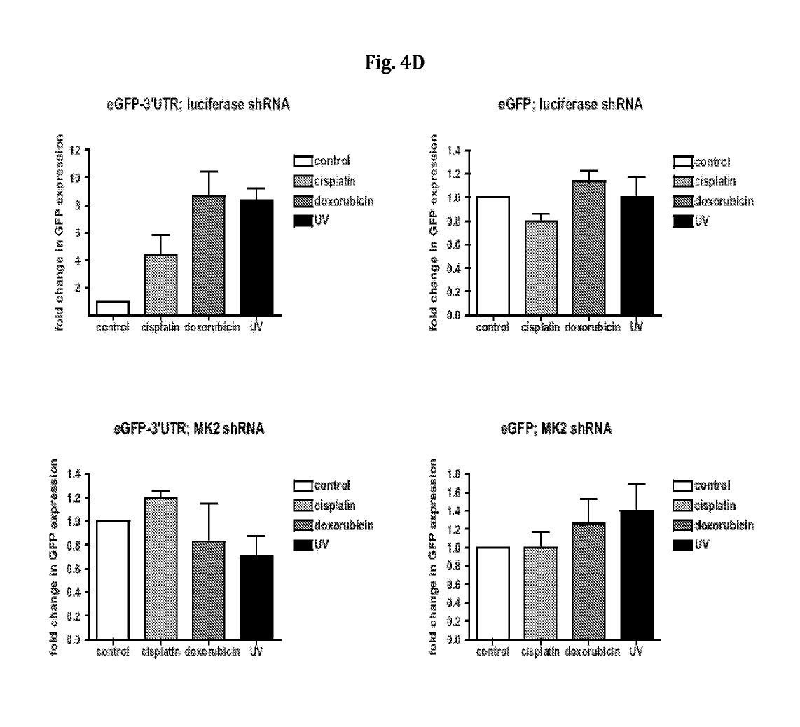

MK2 has previously been implicated for a role in the stabilization of mRNAs containing AU-rich elements (AREs) in the 3′ UTR (Gaestel et al., Nat. Rev. Mol. Cell Biol. 7: 120-130, 2006). In order to identify likely substrates of MK2 that are critical for its late cytoplasmic checkpoint-maintaining function, a number of molecules potentially involved in cell-cycle control were surveyed for the presence of 3′ AREs. Gadd45a, a cell-cycle regulator known to be induced after DNA damage in both a p53-dependent and -independent manner, emerged as a likely candidate among the molecules initially identified. Gadd45a mRNA is rapidly upregulated following doxorubin-induced DNA damage, and accumulation of this mRNA was almost completely abolished when cells were depleted of MK2 (FIG. 4A). However, upregulation of Gadd45a mRNA following genotoxic stress could be restored in MK2 knockdown cells if they were complemented with a cytoplasmic-localized form of Chk1

An RNAi approach was used to directly investigate the functional importance of DNA damage-induced Gadd45a induction (FIG. 4B). These data show that knockdown of Gadd45a in MK2-containing cells was found to result in premature collapse of both the doxorubicin-induced intra-S and G₂/M checkpoints by 30 hours after treatment (FIG. 4B), phenocopying the loss of checkpoint function in MK2 knockdown cells. These data indicate that Gadd45a is a critical MK2 target for checkpoint function.

The 3′UTR of Gadd45a is heavily AU rich and contains numerous AREs, making posttranscriptional regulation through this part of the mRNA likely. Under resting conditions, Gadd45a was shown to be actively degraded via a mechanism involving the 3′UTR (Lal et al., Mol. Cell. 22:117-128, 2006). To investigate whether p38/MK2-dependent pathway(s) actively stabilize Gadd45a mRNA levels through its 3′UTR, reporter constructs in which the GFP coding sequence was fused to the Gadd45a 3′UTR were generated (FIGS. 4C and 4D). The GFP-3′UTR fusion construct, or GFP alone, was expressed in HeLa cells expressing either control or MK2-specific shRNA hairpins. The basal levels of GFP protein were markedly lower in cells expressing the Gadd45a 3′UTR-chimeric mRNA than in cells expressing the unfused GFP mRNA (FIGS. 4C and 4D). Cells expressing the 3′UTR chimeric GFP showed substantial induction of GFP following doxorubicin and UV treatment (˜9-fold) and milder upregulation after cisplatin exposure (˜4-fold). In marked contrast, the expression levels of the unfused GFP control protein remained unchanged. These results are consistent with regulation of Gadd45a mRNA levels through a posttranscriptional mechanism involving the 3′ UTR.

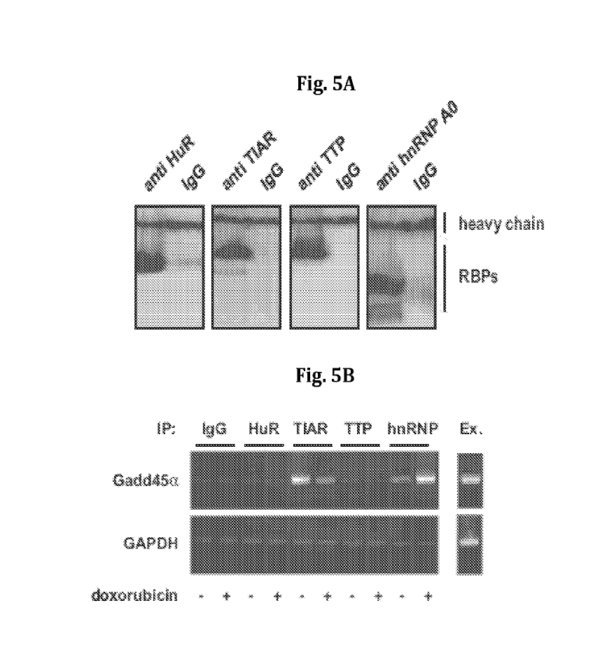

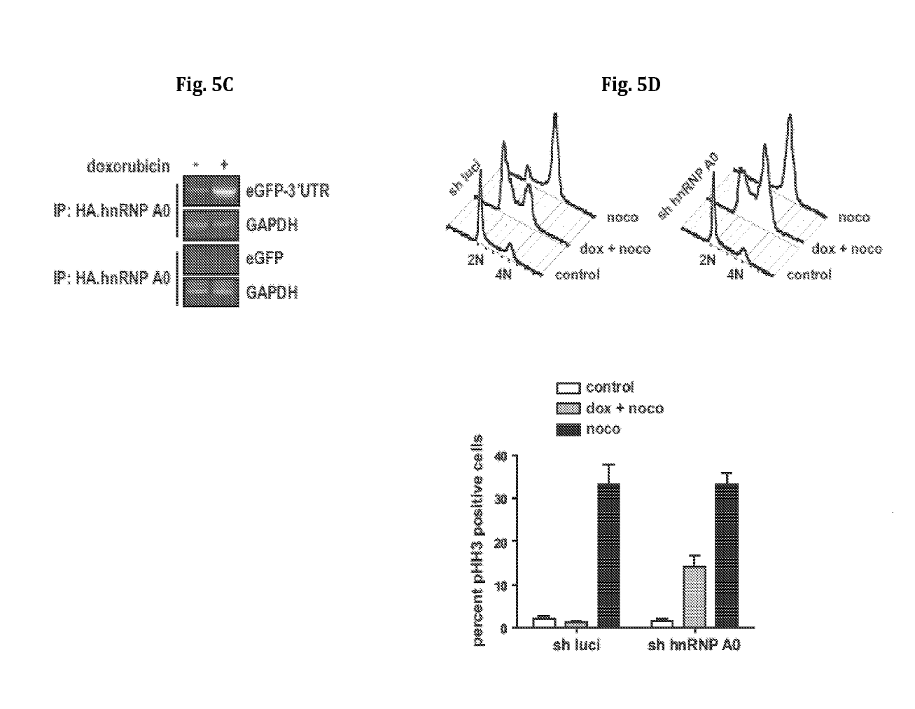

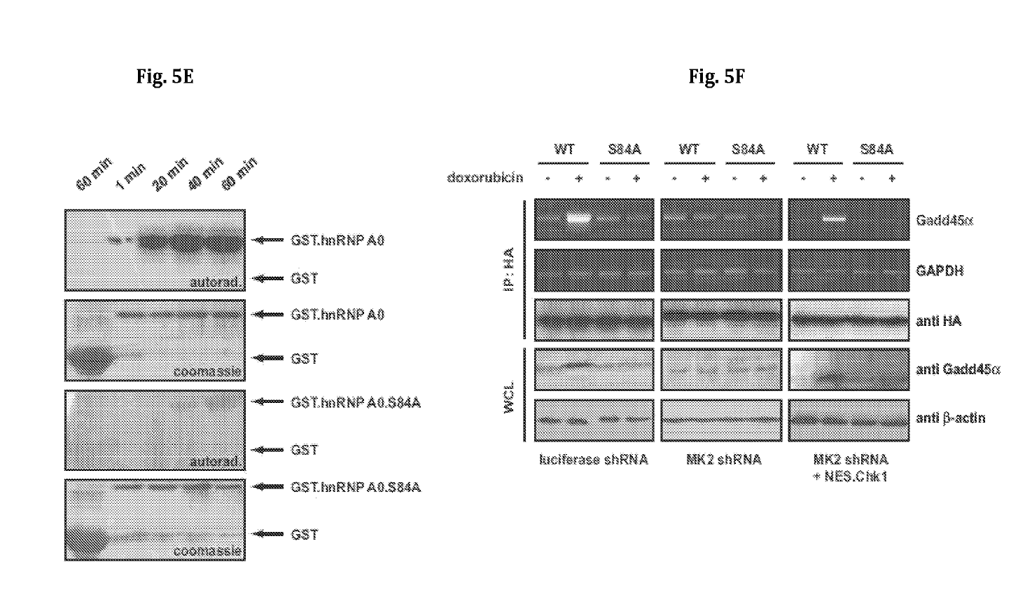

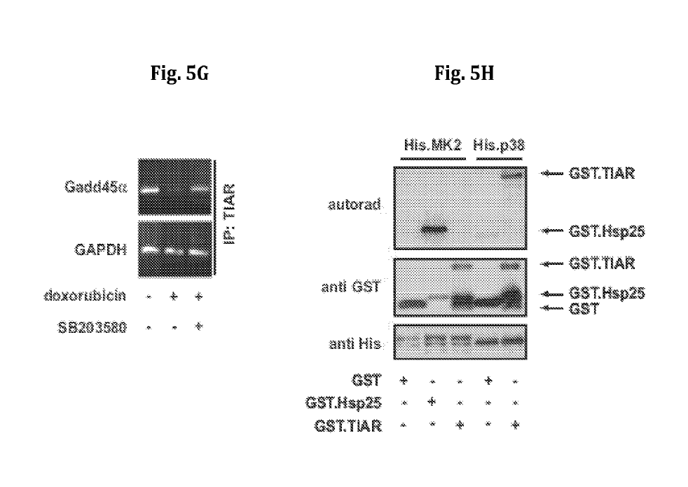

To determine whether MK2 might impose control over the Gadd45a mRNA through phosphorylation of RNA-binding proteins (RBPs), Scansite (Obenauer et al., Nucleic Acids Res. 31:3635-3641, 2003) was used to identify ARE-binding RBPs containing the MK2 consensus phosphorylation motif (Manke et al., Mol. Cell 17:37-48, 2005). This analysis identified HuR, TTP, TIAR, and hnRNPA0 as candidate MK2 substrates. RNA-IP followed by RT-PCR was used to investigate which of these proteins were bound to Gadd45a mRNA (FIGS. 5A an 5B).

RNA-IP was performed by lysing the cells in 0.5 mL of ice-cold RNA lysis buffer (110 mM CH₃COOK, 2 mM Mg[CH₃COO]₂, 10 mM HEPES [pH 7.4], 200 mM KCl, 0.5% NP-40, 40 μL/mL complete protease inhibitor [Roche], and 50 units/mL RNAsin) per 10-cm dish on ice. The resulting extracts were homogenized using a 26.5 gauge needle, cleared by centrifugation at 4200 rpm for 10 minutes and incubated with antibody-coated beads for 2 hours. After extensive washing in TBS, the beads were eluted with 0.5 mL elution buffer (10 mM Tris-HCl [pH 7.5], 1 mM EDTA, 1% SDS, Proteinase K) for 1 hour at 37° C. with rocking. The eluted material was phenol/chloroform extracted followed by CH₃COONH₄/isopropanol precipitation. The pellets were washed in 70% ethanol, resuspended in H₂O, and DNase treated, and reverse-transcribed using MMLV RT (Ambion) with random hexamer primers. Primers for the subsequent PCR were as follows: 5′-GATGCCCTGGAGGAAGTGCT-3′ (forward; SEQ ID NO: 27) and 5′-AGCAGGCACAA CACCACGTT-3′ (reverse; SEQ ID NO: 28) for Gadd45a and 5′-TGCACCACCAA CTGCTTAGC-3′ (forward; SEQ ID NO: 29) and 5′-GGCATGGACTGTGGTCATGAG-3′ (reverse; SEQ ID NO: 30) for GAPDH amplification. The above IP conditions used were optimized to maintain the integrity of endogenous ribonucleoprotein (RNP) complexes.

Following performance of RNA-IP, the subsequent RT-PCR analysis using Gadd45a-specific primers revealed prominent bands only from the TIAR and hnRNPA0 immunoprecipitate. No detectable amplification was seen in the control IgG IP or after IP with antibodies recognizing HuR or TTP. Importantly, a reduction of Gadd45a mRNA binding to TIAR was observed following doxorubicin exposure (FIG. 5B). This observation is consistent with a known role of TIAR in translational inhibition. On the other hand, Gadd45a mRNA levels in complex with hnRNPA0 appeared to be substantially increased after doxorubicin treatment (FIG. 5B). Low-level but equal PCR products for GAPDH mRNA were seen in all of the IP samples tested; these signals likely represent the background binding of cellular mRNA to the IP reagents and hence served as loading control.

Cells were transfected with the GFP-3′UTR hybrid construct and hnRNPA0 IP was performed in order to directly explore whether hnRNPA0 binds specifically to the 3′UTR of Gadd45a mRNA. As observed for endogenous Gadd45a mRNA, treatment with doxorubicin induced a robust binding of eGFP mRNA fused to the Gadd45a-3′UTR (FIG. 5C). These data strongly indicate that hnRNPA0 binds to endogenous Gadd45a mRNA via the 3′UTR in a DNA damage-inducible manner.

In additional set of experiments, an RNAi approach was used to determine if hnRNPA0 was involved in the MK2-dependent checkpoint. The knockdown of hnRNPA0 resulted in a substantial impairment of the intra-S and G₂ checkpoint arrest following doxorubicin treatment (FIG. 5D), recapitulating, in part, the effect observed in cells lacking MK2 or Gadd45a. hnRNPA0 has a single optimal phosphorylation site for MK2 at serine-84 (Rousseau et al., EMBO J. 21:6505-6514, 2002) (FIG. 5E). In order to test whether hnRNPA0 phosphorylation on serine-84 is required for mRNA binding, HeLa cells were transfected with HA-tagged hnRNPA0 or with a mutant form of hnRNPA0 in which serine-84 was replaced with Ala (FIG. 5F). The resulting transfected cells were either treated with doxorubicin or left untreated and lysed 12 hours later. hnRNPA0 was recovered by immunoprecipitation with an anti-HA-antibody, followed by RT-PCR analysis for bound Gadd45a mRNA using specific primers. The results show a prominent Gadd45a mRNA band from hnRNPA0 wild-type transfected MK2-proficient cells exposed to doxorubicin. In contrast, the interaction between hnRNPA0 and Gadd45a mRNA was almost entirely lost in cells transfected with the Ser-84 alanine mutant (FIG. 5F, left panels). Similarly, no binding between hnRNPA0 and Gadd45a mRNA was seen in MK2-depleted cells (FIG. 5F, middle panels). Importantly, expression of a cytoplasmic-targeted form of Chk1 restored DNA damage-stimulated hnRNPA0-Gadd45a mRNA binding and Gadd45a protein expression in MK2-depleted cells (FIG. 5F, right panels), further supporting the notion that cytoplasmic checkpoint kinase activity is required for functional cell-cycle checkpoint control. These data strongly suggest a model in which long-term maintenance of DNA damage checkpoints involves MK2-dependent phosphorylation of hnRNPA0, stimulating its binding to the Gadd45a 3′UTR, with subsequent Gadd45a mRNA stabilization.

In contrast to hnRNPA0, MK2-dependent phosphorylation of TIAR was not observed. However, a strong direct phosphorylation of TIAR by p38 was observed in vitro (FIG. 5G), together with a marked decreased in release of Gadd45a mRNA from TIAR following DNA damage in vivo if the cells were treated with the p38 inhibitor SB203580 (FIG. 5H). These data indicate that the combined actions of p38 and MK2 are responsible for the release of Gadd45a mRNA from TIAR and its accumulation and stabilization on hnRNPA0.

### Example 4

### MK2-Mediated Phosphorylation of PARN is Required to Prevent Gadd45a mRNA Degradation after Genotoxic Stress

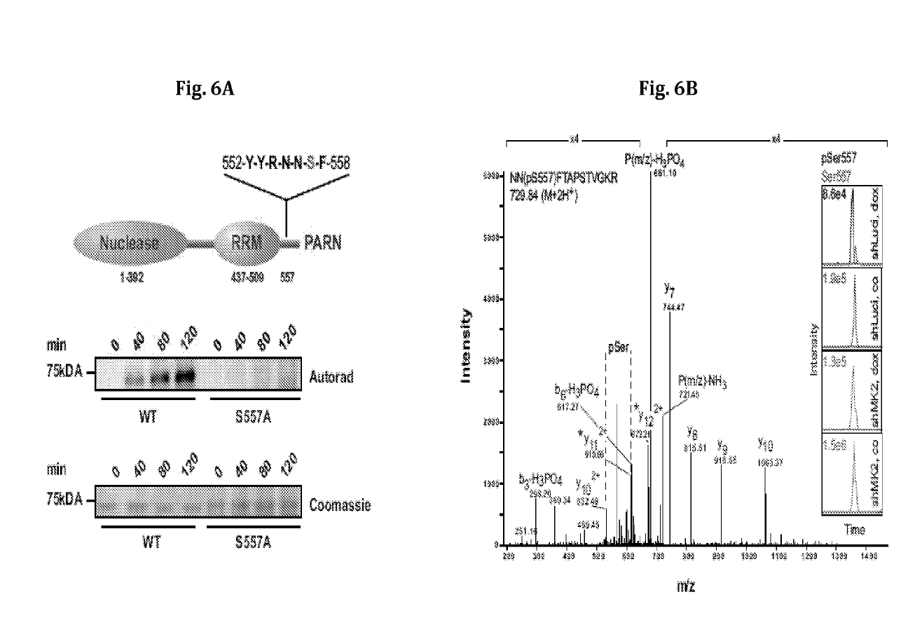

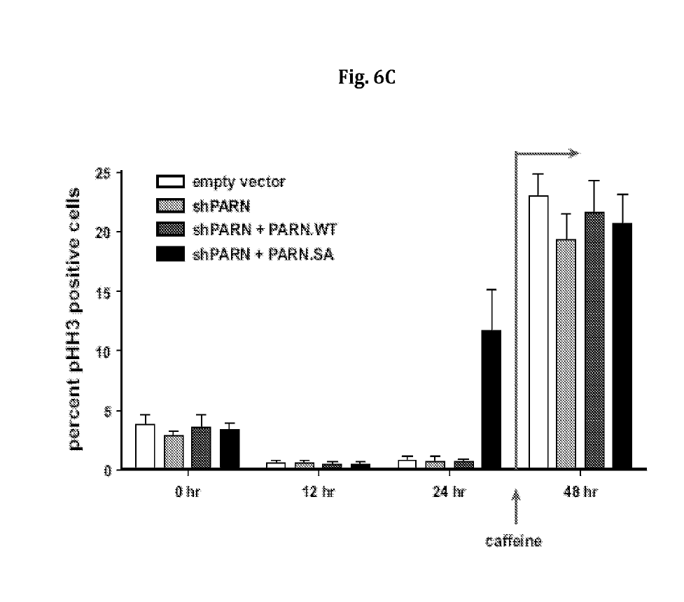

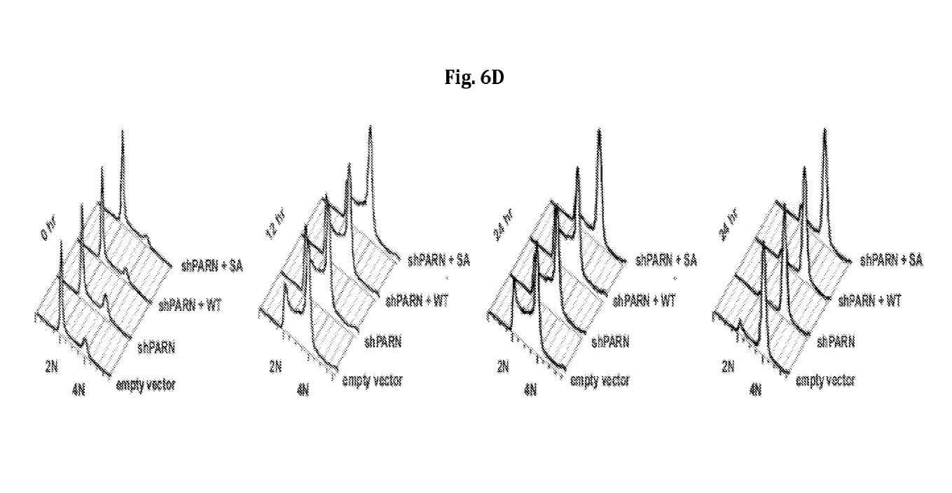

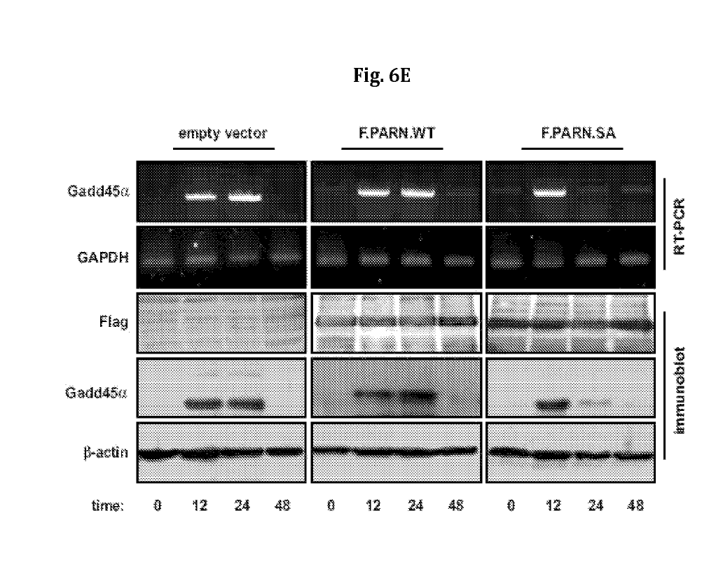

In order to identify further substrates of MK2 that might be involved in checkpoint signaling through posttranscriptional control of gene expression, previously published computer algorithms Scansite and NetworkKIN (Linding et al., Cell 129:1415-1426, 2007; Obenauer et al., Nucleic Acids Res. 31:3635-3641, 2003) were used to search mass spectrometry databases for phosphorylated proteins likely to be substrates for the p38/MK2 pathway. This analysis revealed poly-A ribonuclease (PARN) as a potential MK2 target. Recombinant wild-type PARN was strongly phosphorylated by MK2 in vitro (FIG. 6A); however, PARN in which serine-557 was mutated to alanine showed dramatically reduced levels of phosphorylation, confirming that PARN can serve as a direct substrate for MK2, and demonstrating that serine-557 is the dominant MK2 phosphorylation site.

To investigate whether MK2 phosphorylates PARN in vivo in response to genotoxic stress, USO2 cells expressing either a luciferase or an MK2-specific shRNA were treated with 10 mM doxorubicin. Four hours following doxorubicin, endogenous PARN was affinity purified from cell lysates and analyzed by mass spectrometry. As shown in FIG. 6B, serine-557 phosphorylated PARN peptides were not detected in untreated USO2 cells expressing the luciferase control shRNA. In marked contrast, serine-557 phosphorylated peptides were readily detected when these cells were treated with doxorubicin. This DNA-damage induced phosphorylation event was completely abolished in MK2-depleted cells, indicating that MK2 is the in vivo kinase directly responsible for genotoxic stress-induced PARN serine-557 phosphorylation.

In order to determine whether MK2-dependent phosphorylation of PARN at serine-557 plays a role in checkpoint control, RNAi was used to deplete endogenous PARN from HeLa cells, and these cells were completed with RNAi-resistant FLAG-tagged wild-type PARN or the serine-557 to alanine PARN mutant. The cells were treated with low dose (0.1 mM) doxorubicin for 1 hour, the drug washed out, and the spontaneous escape of cells from the doxorubicin-induced cell cycle checkpoints monitored 12 and 24 hours later using the nocodazole mitotic-trap assay as used in FIG. 1. Control cells expressing either an empty vector or PARN shRNA mounted and maintained a robust doxorubicin-induced cell cycle arrest 12 and 24 hours later, indicated by an accumulation of cells with a 4N DNA content that stained largely negative for the mitotic marker pHH3 (FIGS. 6C and 6D). A similar pattern was observed in PARN-depleted cells that were complemented with exogenous wild-type PARN. In contrast, cells depleted of endogenous PARN and complemented with the serine-557 to alanine mutant could initiate, but were unable to maintain a prolonged doxorubicin-induced cell cycle arrest, indicated by the accumulation of 11.7% pHH3-positive cells 24 hours after the addition of doxorubicin. As a control, 5 mM caffeine was then added to each of the plates following the 24-hour measurement to inhibit ATM/ATR/DNA-PK and chemically inactivate the DNA damage checkpoint. This resulted in similar checkpoint release from all the PARN-manipulated cells, verifying that the cells after each treatment are equally viable and competent to enter mitosis. These observations demonstrate that phosphorylation of PARN on serine-557 by MK2 is required for proper cell-cycle checkpoint maintenance, and suggest that phosphorylation of PARN may alter the degradation of specific RNAs involved in checkpoint control.

To further investigate whether the role of MK2-mediated PARN phosphorylation in cell-cycle control was mediated through posttranscriptional control of Gadd45a mRNA, lysates from the PARN-depletion/complementation experiments above were assayed for Gadd45a mRNA levels. In response to doxorubicin, all cells showed robust upregulation of Gadd45a mRNA at 12 hours after treatment (FIG. 6E). In PARN-depleted cells complemented with the nonphosphorylatable PARN mutant showed a precipitous decline in Gadd45a mRNA levels back to baseline values between 12 and 24 hours, and only a miniscule amount of protein at 24 hours, consistent with the premature checkpoint collapse previously observed. Upon forced cell cycle re-entry by caffeine addition, Gadd45a mRNA and protein levels dropped below the limits of detection following all of the cell treatments (FIG. 6E, 48 hour lanes). Together, these data show that MK2 phosphorylation of PARN on serine-557 in response to genotoxic stress is critical for maintenance of both Gadd45a mRNA and protein expression in response to DNA damage.

### Example 5

### A Gadd45a-Mediated Positive Feedback Loop is Required for Sustained Long-Term MK2 Activity to Suppress Cdc25B and C-Driven Mitotic Re-Entry after Genotoxic Stress

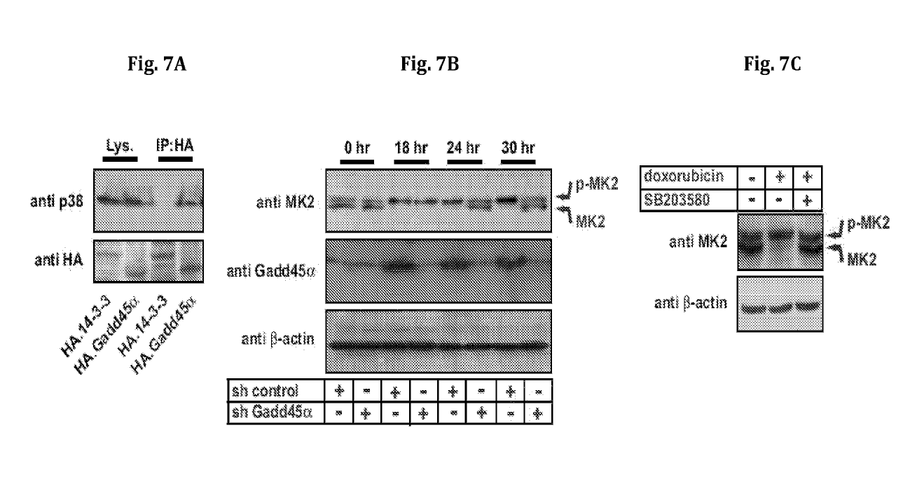

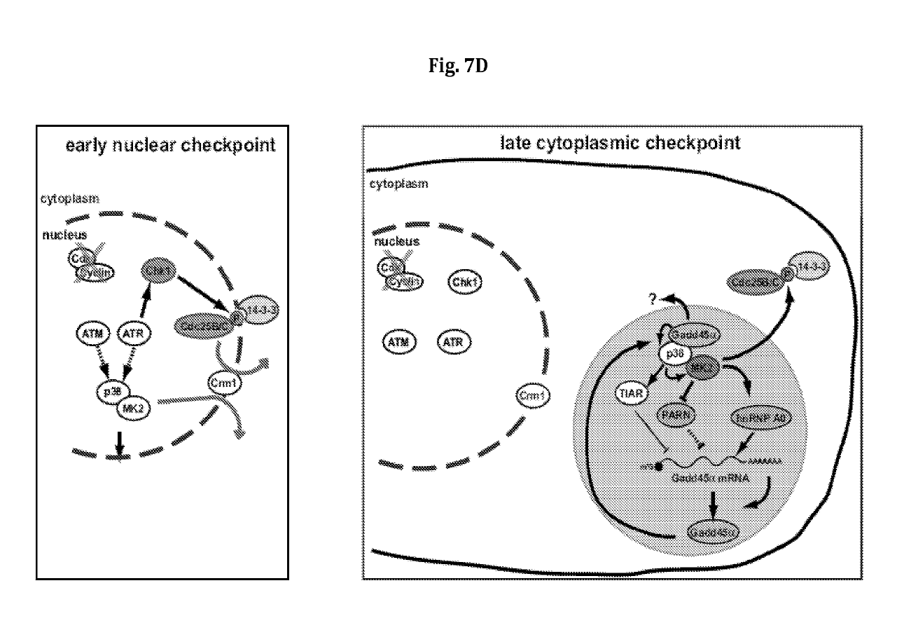

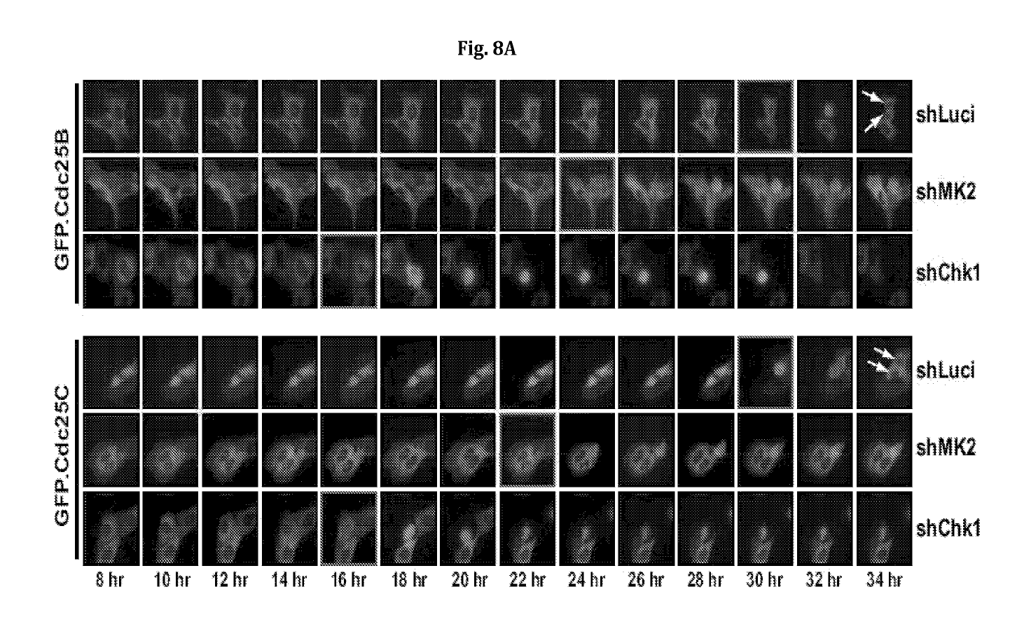

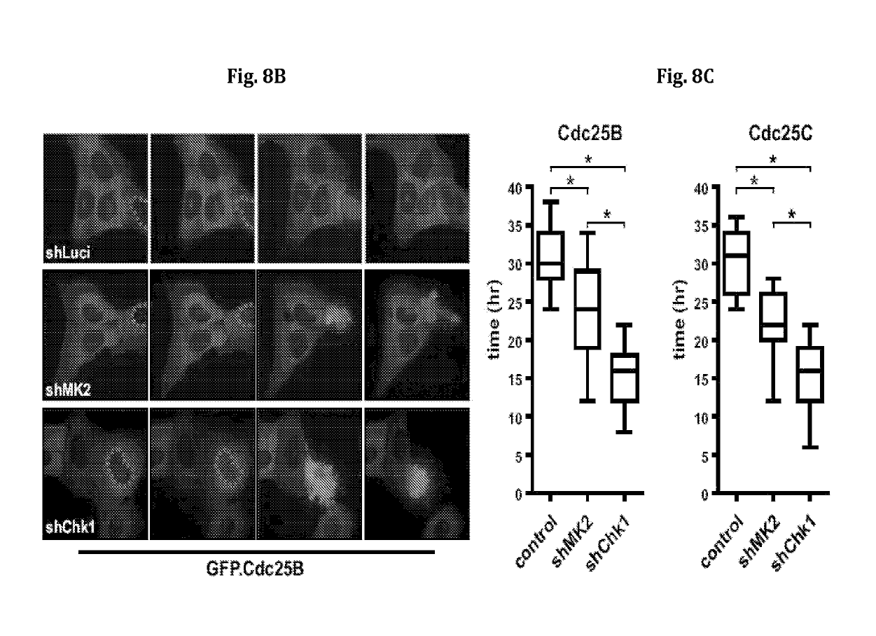

Additional experiments were performed to determine whether the MK2-dependent regulation of Gadd45a that was required for maintenance of late cell-cycle arrest after DNA damage was related to the MK2-dependent phosphorylation and inactivation of Cdc25B and C. An initial set of experiments demonstrated that detectable direct interactions between HA-tagged Gadd45a and the endogenous kinases, MKK3 or MKK6, two known upstream regulators of p38 that response to inflammatory stimuli or UV irradiation (data not shown). A strong interaction between Gadd45a and p38 was observed (FIG. 7A). In an additional set of experiments, Gadd45a was depleted using RNAi. The data from these experiments show a loss of p38-dependent MK2 phosphorylation specifically at late times after DNA damage (FIGS. 7B and 7C). Importantly, the data indicate that loss of Gadd45a has little if any effect on MK2 activation at early times. These observations suggest a model in which the initial activation of MK2 after genotoxic stress does not depend on Gadd45a, but subsequent p38/MK2-dependent stabilization of Gadd45a, through phosphorylation of TIAR, PA RN, and hnRNPA0, becomes required for maintaining the phosphorylated and active form of MK2 at late times (FIG. 7D). To further investigate this model, the subcellular localization of cdc25B/C along with phenotypic responses was examined in cells in which this feedback loop was disrupted (FIG. 8) In these experiments, stable cell lines expressing GFP-tagged versions of cdc25B/C were generated and subsequently infected with lentiviral shRNAs targeting MK2, Chk1, or luciferase (control). The cells were treated with low dose (0.1 mM) doxorubicin for 30 minutes and the subcellular localization of cdc25B/C monitored in live cells by time-lapse fluorescence microscopy.

For live-cell imaging, the cells were grown in four chambered glass-bottom slides from Nunc. Images were obtained using a DeltaVision Core live-cell microscopy imaging system maintained at 37° C. and 5% CO₂ (Applied Precision) and equipped with a Coolsnap CCD camera. Improvision deconvolution and softWoRx software packages were used for image analysis.

In control cells, cdc25B/C lose their cytoplasmic sequestration and first appear in the nucleus at 30.3±3.9 hours and 30.3±3.7 hours, respectively, after this low-level DNA-damaging treatment (FIGS. 8A and 8B). This nuclear entry was followed by a cytologically normal mitotic cell division that occurred ˜2 hours later, producing two intact daughter cells (FIG. 8A, top row of upper and lower panels, arrows indicate the two daughter cells). In Chk1-depleted cells, nuclear entry of Cdc25B/C after this treatment occurred much more rapidly, with a mean onset of 15.4±3.9 hours and 15.3±4.5 hours, respectively (FIGS. 8A and 8C). This premature nuclear entry was invariably followed by catastrophic mitosis resulting in apoptosis, indicated by prominent membrane blebbing and nuclear pyknosis and disintegration (FIG. 8B and data not shown). In contrast, in MK2-depleted cells, nuclear entry of Cdc25B/C was delayed relative to Chk1-depleted cells, but occurred significantly earlier than in control cells, with a mean appearance time of 23.9±6.0 hours and 22.4±14.1 hours, respectively (FIGS. 8A and 8C). This time corresponds exactly to the time when MK2 activity declines in the absence of Gadd45a (FIG. 7B), implying that the positive feedback loop involving MK2-dependent stabilization of Gadd45a, followed by Gadd45a-dependent sustainment of MK2 activity, is critical for prolonged Cdc25 inhibition and maintenance of a G₂ arrest. Together, these data suggest that the p38/MK2/Gadd45a/p38 positive feedback loop is essential to allow cells to recover from the doxorubicin-induced DNA damage before committing to the next mitotic cell division.

All publications, patents, and patent applications mentioned in the above specification are hereby incorporated by reference. In addition, U.S. Patent Application Publication Nos. 2009/0010927, 2005/0196808, 2010/0030543, 2006/0115453, 2009/0181468, 2006/0052951, and 2009/0143997, are each hereby incorporated by reference in their entirety. Further, International Patent Application Publication Nos. WO 05/115454, WO 04/046317, WO 06/053315, and WO 05/115454, are each hereby incorporated by reference in their entirety. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of diagnosing a chemotherapy-resistant cancer in a patient comprising the steps of:
(i) measuring one or more feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat-shock protein-27 (hsp27), levels of phosphorylated hnRNPA0, levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and
(ii) determining from the measurements in step (i) whether said cancer cell(s) in said patient has one or more feature(s) of an activated MK2 signaling pathway selected from the group of: increased cytoplasmic MK2 protein localization, decreased nuclear MK2 protein localization, increased phosphorylation of total MK2 protein, increased levels of phosphorylated MK2 protein in the cytoplasm or nucleus, increased levels of phosphorylated hsp27, increased levels of phosphorylated hnRNPA0, increased levels of phosphorylated PARN, increased levels of phosphorylated TIAR, increased levels of phosphorylated cdc25B, increased levels of phosphorylated cdc25C, and increased levels of Gadd45a protein or mRNA relative to these features in a control sample;
wherein a cancer cell having one or more said feature(s) of an activated MK2 signaling pathway indicates that said patient has a chemotherapy-resistant cancer.

2. The method of claim 1, further comprising the steps of:
(iii) measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity; and
(iv) determining from the measurements in step (ii) whether said cancer cell(s) in said patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample;
wherein a cancer cell having one or more said feature(s) of an activated MK2 signaling pathway and one or more said feature(s) of an inactivated p53 signaling pathway indicates that said patient has a chemotherapy-resistant cancer.

3-6. (canceled)

7. A method of diagnosing a chemotherapy-sensitive cancer in a patient comprising the steps of:
(i) measuring one or more feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated hnRNPA0, levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and
(ii) determining from the measurements in step (i) whether said cancer cell(s) in said patient has one or more feature(s) of an inactivated MK2 signaling pathway selected from the group of: decreased cytoplasmic MK2 protein localization, increased nuclear MK2 protein localization, decreased phosphorylation of total MK2 protein, decreased levels of phosphorylated MK2 protein in the cytoplasm or nucleus, decreased levels of phosphorylated hsp27, decreased levels of phosphorylated hnRNPA0, decreased levels of phosphorylated PARN, decreased levels of phosphorylated TIAR, decreased levels of phosphorylated cdc25B, decreased levels of phosphorylated cdc25C, and decreased levels of Gadd45a protein or mRNA relative to these features in a control sample;
wherein a cancer cell having one or more said feature(s) of an inactivated MK2 signaling pathway indicates that said patient has a chemotherapy-sensitive cancer.

8. The method of claim 7, further comprising the steps of:
(iii) measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity; and
(iv) determining from the measurements in step (ii) whether said cancer cell(s) in said patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample;
wherein a cancer cell having one or more said feature(s) of an inactivated MK2 signaling pathway and one or more said feature(s) of an inactivated p53 signaling pathway indicates that said patient has a chemotherapy-sensitive cancer.

9-12. (canceled)

13. A method of treating a cancer patient diagnosed as having a chemotherapy-resistant cancer by the method of claim 1, comprising the step of administering to the patient one or more MK2 inhibitor(s).

14. The method of claim 13, further comprising the administration of one or more chemotherapeutic agent(s) to the patient.

15. The method of claim 14, wherein the chemotherapeutic agent induces DNA damage.

16-21. (canceled)

22. The method of claim 13, wherein the MK2 inhibitor is a peptide and wherein the peptide contains a covalently-linked moiety capable of translocating across a biological membrane.

23. The method of claim 22, wherein the moiety comprises a penetratin peptide or TAT peptide.

24. A method of treating a cancer patient diagnosed as having a chemotherapy-sensitive cancer by the method of claim 7, comprising the step of administering to the patient one or more chemotherapeutic agent(s).

25-27. (canceled)

28. A method of reducing the severity of one or more symptom(s) of cancer in a patient comprising the steps of:
(i) measuring one or more feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and
(ii) determining from the measurements in step (i) whether said cancer cell(s) in said patient has one or more feature(s) of an activated MK2 signaling pathway selected from the group of: increased cytoplasmic MK2 protein localization, decreased nuclear MK2 protein localization, increased phosphorylation of total MK2 protein, increased levels of phosphorylated MK2 protein in the cytoplasm or nucleus, increased levels of phosphorylated hsp27, increased levels of phosphorylated hnRNPA0, increased levels of phosphorylated PARN, increased levels of phosphorylated TIAR, increased levels of phosphorylated cdc25B, increased levels of phosphorylated cdc25C, and increased levels of Gadd45a protein or mRNA relative to these features in a control sample; and
(iii) administering to a patient determined to have a cancer cell having one or more said feature(s) of an activated MK2 signaling pathway one or more MK2 inhibitor(s) for a time and in an amount sufficient to reduce the severity of one or more symptom(s) of cancer in the patient.

29. The method of claim 28, further comprising the steps of:
(iv) measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity;
(v) determining from the measurements in step (iv) whether said cancer cell(s) in said patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; and
(vi) administering to a patient determined to have a cancer cell having one or more said feature(s) of an activated MK2 signaling pathway and one or more said feature(s) of a defective p53 pathway one or more MK2 inhibitor(s) for a time and in an amount sufficient to reduce the severity of one or more symptom(s) of cancer in the patient.

30. The method of claim 28, wherein step (iii) further comprises the administration of one or more chemotherapeutic agent(s) to the patient.

31. The method of claim 29, wherein step (vi) further comprises the administration of one or more chemotherapeutic agent(s) to the patient.

32-33. (canceled)

34. The method of claim 28, wherein the control sample in step (ii) is a non-cancerous cell or a cell untreated with a genotoxic agent.

35-37. (canceled)

38. The method of claim 28, wherein the MK2 inhibitor is a small molecule, siRNA molecule, nuclease base inhibitor or peptide.

39-40. (canceled)

41. The method of claim 28, wherein the MK2 inhibitor is a peptide comprising the amino acid sequence of [L/F/I]XR[Q/S/T]L[S/T][hydrophobic] (SEQ ID NO: 5), wherein said peptide comprises no more than 50 amino acids.

42. The method of claim 41, wherein the MK2 inhibitor is a peptide comprising the amino acid sequence of LQRQLSI (SEQ ID NO: 6).

43. The method of claim 41, wherein the peptide contains a covalently-linked moiety capable of translocating across a biological membrane.

44. The method of claim 43, wherein the moiety comprises a penetratin peptide or TAT peptide.

45. (canceled)

46. A method of reducing the severity of one or more symptoms of cancer in a patient comprising the steps of:
(i) measuring one or more feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA;
(ii) determining from the measurements in step (i) whether said cancer cell(s) in said patient has one or more feature(s) of an inactivated MK2 signaling pathway selected from the group of: decreased cytoplasmic MK2 protein localization, increased nuclear MK2 protein localization, decreased phosphorylation of total MK2 protein, decreased levels of phosphorylated MK2 protein in the cytoplasm or nucleus, decreased levels of phosphorylated hsp27, decreased levels of phosphorylated hnRNPA0, decreased levels of phosphorylated PARN, decreased levels of phosphorylated TIAR, decreased levels of phosphorylated cdc25B, decreased levels of phosphorylated cdc25C, and decreased levels of Gadd45a protein or mRNA relative to these features in a control sample; and
(iii) administering to a patient determined to have a cancer cell having one or more said feature(s) of an inactivated MK2 signaling pathway one or more chemotherapeutic agent(s) for a time and in an amount sufficient to reduce the severity of one or more symptom(s) of cancer in the patient.

47. The method of claim 46, further comprising the steps of:
(iv) measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity;
(v) determining from the measurements in step (ii) whether said cancer cell(s) in said patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample; and
(vi) administering to a patient determined to have a cancer cell having one or more said feature(s) of an inactivated MK2 signaling pathway and one or more said feature(s) of an inactivated p53 pathway one or more chemotherapeutic agent(s) for a time and in an amount sufficient to reduce the severity of one or more symptom(s) of cancer in the patient.

48. The method of claim 46, wherein the chemotherapeutic agent induces DNA damage.

49-54. (canceled)

55. A method of identifying a cancer patient that may selectively benefit from the administration of one or more MK2 inhibitor(s) or the administration of the combination of one or more MK2 inhibitor(s) and one or more chemotherapeutic agent(s) comprising the steps of:
(i) measuring one or more feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phosphorylated poly(A)-specific ribonuclease (PARN), and levels of growth arrest and DNA-damage-inducible-45A (Gadd45a) protein or mRNA; and
(ii) determining from the measurements in step (i) whether said cancer cell(s) in said patient has one or more feature(s) of an activated MK2 signaling pathway selected from the group of: increased cytoplasmic MK2 protein localization, decreased nuclear MK2 protein localization, increased phosphorylation of total MK2 protein, increased levels of phosphorylated MK2 protein in the cytoplasm or nucleus, increased levels of phosphorylated hsp27, increased levels of phosphorylated hnRNPA0, increased levels of phosphorylated PARN, increased levels of phosphorylated TIAR, increased levels of phosphorylated cdc25B, increased levels of phosphorylated cdc25C, and increased levels of Gadd45a protein or mRNA relative to these features in a control sample;
wherein a patient having one or more said feature(s) of an activated MK2 signaling pathway is identified as a cancer patient that may selectively benefit from the administration of one or more MK2 inhibitor(s) or the administration of the combination of one or more MK2 inhibitor(s) and one or more chemotherapeutic agent(s).

56. The method of claim 55, further comprising the steps of:
(iii) measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity; and
(iv) determining from the measurements in step (ii) whether said cancer cell(s) in said patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample;
wherein a patient having one or more said feature(s) of an activated MK2 signaling pathway and one or more said feature(s) of an inactivated p53 pathway is identified as a cancer patient that may selectively benefit from the administration of one or more MK2 inhibitor(s) or the administration of the combination of one or more MK2 inhibitor(s) and one or more chemotherapeutic agent(s).

57-62. (canceled)

63. A method of identifying a cancer patient that may selectively benefit from the administration of dosage of one or more chemotherapeutic agent(s) comprising the steps of:
(i) measuring one or more the feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated heterogeneous nuclear ribonucleoprotein A0 (hnRNPA0), levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and
(ii) determining from the measurements in step (i) whether said cancer cell(s) in said patient has one or more feature(s) of an inactivated MK2 signaling pathway selected from the group of: decreased cytoplasmic MK2 protein localization, increased nuclear MK2 protein localization, decreased phosphorylation of total MK2 protein, decreased levels of phosphorylated MK2 protein in the cytoplasm or nucleus, decreased levels of phosphorylated hsp27, decreased levels of phosphorylated hnRNPA0, decreased levels of phosphorylated PARN, decreased levels of phosphorylated TIAR, decreased levels of phosphorylated cdc25B, decreased levels of phosphorylated cdc25C, and decreased levels of Gadd45a protein or mRNA relative to these features in a control sample;
wherein a patient having one or more said feature(s) of an inactivated MK2 signaling pathway is identified as a cancer patient that may selectively benefit from the administration of one or more chemotherapeutic agent(s).

64. The method of claim 63, further comprising the steps of:
(iii) measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity; and
(iv) determining from the measurements in step (ii) whether said cancer cell(s) in said patient has one or more feature(s) of an inactivated p53 signaling pathway selected from the group of: decreased p53 mRNA or protein levels, expression of a mutant or truncated p53 with decreased expression or activity, and decreased p21 expression or activity relative to these features in a control sample;
wherein a patient having one or more said feature(s) of an inactivated MK2 signaling pathway and one or more said feature(s) of an inactivated p53 pathway is identified as a cancer patient that may selectively benefit from the administration of one or more chemotherapeutic agent(s).

65-70. (canceled)

71. A kit for diagnosing a chemotherapy-resistant or chemotherapy-sensitive cancer in a patient comprising: one or more reagent(s) capable of measuring one or more feature(s) in a cancer cell(s) from said patient selected from the group consisting of: cytoplasmic or nuclear MAPKAP kinase-2 (MK2) protein localization, phosphorylation of total MK2 protein, levels of phosphorylated MK2 protein in the cytoplasm or nucleus, levels of phosphorylated heat shock protein-27 (hsp27), levels of phosphorylated hnRNPA0, levels of phosphorylated poly(A)-specific ribonuclease (PARN), levels of phosphorylated TIA-1 related protein (TIAR), levels of phosphorylated cell division cycle 25B (cdc25B), levels of phosphorylated cell division cycle 25C (cdc25C), and levels of growth arrest and DNA-damage-inducible-45A (Gadd45a) protein or mRNA; and
b) instructions for using the reagents of (a) to determine the presence of a chemotherapy-resistant or chemotherapy-sensitive cancer in said patient.

72. The kit of claim 71, further comprising:
c) one or more reagent(s) capable of measuring one or more feature(s) in a cancer cell(s) from said patient selected from the group consisting of: tumor protein-53 (p53) mRNA or protein levels, expression of a mutant or truncated p53 with decreased expression or activity, and cyclin-dependent kinase inhibitor 1 (p21) expression or activity; and
d) instructions for using the reagents of (a) and (b) to determine the presence of a chemotherapy-resistant or chemotherapy-sensitive cancer in said patient.

73. The kit of claim 71, wherein said one or more reagent(s) in (a) are selected from the group consisting of: an antibody that binds phosphorylated, nonphosphorylated, or total MK2 protein; an antibody that binds phosphorylated, nonphosphorylated, or total hsp27; an antibody that binds to phosphorylated, nonphosphorylated, or total hnRNPA0; an antibody that binds to phosphorylated, nonphosphorylated, or total PARN; an antibody that binds to phosphorylated, nonphosphorylated, or total TIAR; an antibody that binds to Gadd45a; an antibody that binds to phosphorylated, nonphosphorylated, or total cdc25B; an antibody that binds to phosphorylated, nonphosphorylated, or total cdc25C; an oligonucleotide comprising a sequence complementary to a nucleic acid sequence encoding Gadd45a protein; and one or more nucleic acid primer(s) complementary to a sequence in Gadd45a mRNA.

74. The kit of claim 72, wherein said one or more reagent(s) in (c) are selected from the group consisting of: an antibody binding to p53 protein; an oligonucleotide comprising a sequence complementary to a nucleic acid sequence encoding a wild type p53 protein; one or more nucleic acid primer(s) complementary to a nucleic acid sequence encoding a wild type p53 protein; an oligonucleotide comprising a sequence complementary to a nucleic acid sequence encoding a mutant or truncated p53 protein; one or more nucleic acid primer(s) complementary to a nucleic acid sequence encoding a mutant or truncated p53 protein; and an antibody that binds to p21.

75. The kit of claim 74, wherein the nucleic acid sequence encoding a wild type, mutant, or truncated p53 is an mRNA or a genomic DNA sequence.


SEQUENCE LISTING (89 sequences)

SEQ ID NO: 1 - DNA, 21 bp, Artificial Sequence
  MK2 siRNA
cgaugcguguugacuaugann

SEQ ID NO: 2 - DNA, 21 bp, Artificial Sequence
  MK2 siRNA
ucauagucaacacgcaucgnn

SEQ ID NO: 3 - DNA, 21 bp, Artificial Sequence
  MK2 siRNA
ugaccaucaccgaguuuaunn

SEQ ID NO: 4 - DNA, 21 bp, Artificial Sequence
  MK2 siRNA
auaaacucggugauggucann

SEQ ID NO: 5 - protein, 7 aa, Artificial Sequence
  MK2 peptide inhibitor consensus sequence
XXRXLXX

SEQ ID NO: 6 - protein, 7 aa, Artificial Sequence
  MK2 inhibitor
LQRQLSI

SEQ ID NO: 7 - DNA, 19 bp, Artificial Sequence
  Target Sequence in MK2
gaccaggcattcacagaaa

SEQ ID NO: 8 - DNA, 19 bp, Artificial Sequence
  Target Sequence in MK2
ttgaccactccttgttata

SEQ ID NO: 9 - DNA, 19 bp, Artificial Sequence
  Target Sequence in MK2
gaccactccttgttataca

SEQ ID NO: 10 - DNA, 19 bp, Artificial Sequence
  Target Sequence in MK2
tgaccatcaccgagtttat

SEQ ID NO: 11 - DNA, 19 bp, Artificial Sequence
  Target Sequence in MK2
tcaccgagtttatgaacca

SEQ ID NO: 12 - DNA, 19 bp, Artificial Sequence
  Target Sequence in MK2
tcaagaagaacgccatcat

SEQ ID NO: 13 - DNA, 19 bp, Artificial Sequence
  Target Sequence in MK2
aagcatccgaaatcatgaa

SEQ ID NO: 14 - DNA, 19 bp, Artificial Sequence
  Target Sequence in MK2
agtatctgcattcaatcaa

SEQ ID NO: 15 - DNA, 19 bp, Artificial Sequence
  Target Sequence in MK2
ctttgaccactccttgtta

SEQ ID NO: 16 - DNA, 19 bp, Artificial Sequence
  Target Sequence in MK2
tttgaccactccttgttat

SEQ ID NO: 17 - DNA, 19 bp, Artificial Sequence
  Target Sequence in MK2
tacggatcgtggatgtgta

SEQ ID NO: 18 - DNA, 19 bp, Artificial Sequence
  Target Sequence in MK2
ggacggtggagaactcttt

SEQ ID NO: 19 - DNA, 19 bp, Artificial Sequence
  Target Sequence in MK2
cttgttatacaccgtacta

SEQ ID NO: 20 - DNA, 19 bp, Artificial Sequence
  Target Sequence in MK2
gacggtggagaactcttta

SEQ ID NO: 21 - DNA, 19 bp, Artificial Sequence
  Target Sequence in MK2
ggagaactctttagccgaa

SEQ ID NO: 22 - DNA, 3608 bp, Homo sapiens
gcggccgcttccccccggccgggcccccgccgccccgcggtccccagagcgccaggcccccggggggagggagggagggcgccgggccggtgggagccagcggcgcgcggtgggacccacggagccccgcgacccgccgagcctggagccgggccgggtcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacatcctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggggtgtcttcatgacaagaacagcgaccaggccacttggctgaccaggttgtgagcagaggattctgtgttcctgtccaaactcagtgctgtttcttagaatccttttattccctgggtctctaatgggaccttaaagaccatctggtatcatcttctcattttgcagaagagaaactgaggcccagaggcggagggcagtctgctcaaggtcacgcagctggtgactggttggggcagaccggacccaggtttcctgactcctggcccaagtctcttcctcctatcctgcgggatcactggggggctctcagggaacagcagcagtgccatagccaggctctctgctgcccagcgctggggtgaggctgccgttgtcagcgtggaccactaaccagcccgtcttctctctctgctcccacccctgccgccctcaccctgcccttgttgtctctgtctctcacgtctctcttctgctgtctctcctacctgtcttctggctctctctgtacccttcctggtgctgccgtgcccccaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattctgccttggttctggccaccccagagtgggagaggctgggaggttgggaggctgtggagagaagtgagcaaggtgctcttgaacctgtgctcattttgcaattttatcagtaatttgacttagagtttttacgaaacctcttttgttgtccttgccccactcctctccaccagacgccttcctctctggatactgcaaaggcttgtggtttgttagagggtatttgtggaaactgtcatagggattgtccctgtgttgtcccatctgccctccctgtttctccacaacagcctggggttgtccccgctggctcacgcgttctgggagctcaaggccaccttggaggaggatgccacgcacttcctctctcggagccctcagacatctccagtgtgccagacaaataggagtgagtgtatgtgtgtgtgtgtgtgtgtgtgtgtgcacacgtgtgtatgagtgcgcagatctgtgcctgggatcgtgcatttgaggggccaggggcaggcagggctgcagagggagacggccctgctggggcttaggaaccttctcccttcttgggtctgccctgcccatactgagcctgccaaagtgcctgggaagcccacccagattctgaaacaggccctctgtggcctgtctctattagctgggttccgggaggcagagaggagtgaccgggcactggcactgcgatcaggaagactggacccccagcccccagggcccccctccccccacttagtgctggtcctaggtcctctgaggcactcatctactgaatgacctctctacttccccttcttgccattattaacccatttttgtttattttccttaaatttttagccatttctccatgggccaccgcccagctcatgtaggtgagcctgggcagcttctgttggcagagcttttgcatttcctgtgtttgtcctgggttctggggcatcagccagctaccccttgtgggcaaaggcagggccacttttgaagtcttccctcagatttccattgtgtggcctggtgggtcagggggagtctttgcaccaaagatgtcctgactttgcccccttgcccatcagccatttgccatcaccccaaacaactcagcttcggggccggtgaggggaggggcctcccccagcacagatgaggagcagctggggtaggctgtctgtgccatggccccccactcccccttcccttggagggagaggtggcaggaatacttcacctttcctctccctcaggggcaggtggtggaggggcgcccagggtcgtctttgtgtatgggggaaggcgctgggtgcctgcagcgcctcccttgtctcagatggtgtgtccagcactcgattgttgtaaactgttgttttgtatgagcgaaattgtctttactaaacagatttaatagttaaaaaaaaaaaaaaaaaaa

SEQ ID NO: 23 - DNA, 3071 bp, Homo sapiens
gcggccgcttccccccggccgggcccccgccgccccgcggtccccagagcgccaggcccccggggggagggagggagggcgccgggccggtgggagccagcggcgcgcggtgggacccacggagccccgcgacccgccgagcctggagccgggccgggtcggggaagccggctccagcccggagcgaacttcgcagcccgtcggggggcggcggggagggggcccggagccggaggagggggcggccgcgggcacccccgcctgtgccccggcgtccccgggcaccatgctgtccaactcccagggccagagcccgccggtgccgttccccgccccggccccgccgccgcagccccccacccctgccctgccgcaccccccggcgcagccgccgccgccgcccccgcagcagttcccgcagttccacgtcaagtccggcctgcagatcaagaagaacgccatcatcgatgactacaaggtcaccagccaggtcctggggctgggcatcaacggcaaagttttgcagatcttcaacaagaggacccaggagaaattcgccctcaaaatgcttcaggactgccccaaggcccgcagggaggtggagctgcactggcgggcctcccagtgcccgcacatcgtacggatcgtggatgtgtacgagaatctgtacgcagggaggaagtgcctgctgattgtcatggaatgtttggacggtggagaactctttagccgaatccaggatcgaggagaccaggcattcacagaaagagaagcatccgaaatcatgaagagcatcggtgaggccatccagtatctgcattcaatcaacattgcccatcgggatgtcaagcctgagaatctcttatacacctccaaaaggcccaacgccatcctgaaactcactgactttggctttgccaaggaaaccaccagccacaactctttgaccactccttgttatacaccgtactatgtggctccagaagtgctgggtccagagaagtatgacaagtcctgtgacatgtggtccctgggtgtcatcatgtacattctgctgtgtgggtatccccccttctactccaaccacggccttgccatctctccgggcatgaagactcgcatccgaatgggccagtatgaatttcccaacccagaatggtcagaagtatcagaggaagtgaagatgctcattcggaatctgctgaaaacagagcccacccagagaatgaccatcaccgagtttatgaaccacccttggatcatgcaatcaacaaaggtccctcaaaccccactgcacaccagccgggtcctgaaggaggacaaggagcggtgggaggatgtcaaggaggagatgaccagtgccttggccacaatgcgcgttgactacgagcagatcaagataaaaaagattgaagatgcatccaaccctctgctgctgaagaggcggaagaaagctcgggccctggaggctgcggctctggcccactgagccaccgcgccctcctgcccacgggaggacaagcaataactctctacaggaatatattttttaaacgaagagacagaactgtccacatctgcctcctctcctcctcagctgcatggagcctggaactgcatcagtgactgaattctgccttggttctggccaccccagagtgggagaggctgggaggttgggaggctgtggagagaagtgagcaaggtgctcttgaacctgtgctcattttgcaattttatcagtaatttgacttagagtttttacgaaacctcttttgttgtccttgccccactcctctccaccagacgccttcctctctggatactgcaaaggcttgtggtttgttagagggtatttgtggaaactgtcatagggattgtccctgtgttgtcccatctgccctccctgtttctccacaacagcctggggttgtccccgctggctcacgcgttctgggagctcaaggccaccttggaggaggatgccacgcacttcctctctcggagccctcagacatctccagtgtgccagacaaataggagtgagtgtatgtgtgtgtgtgtgtgtgtgtgtgtgcacacgtgtgtatgagtgcgcagatctgtgcctgggatcgtgcatttgaggggccaggggcaggcagggctgcagagggagacggccctgctggggcttaggaaccttctcccttcttgggtctgccctgcccatactgagcctgccaaagtgcctgggaagcccacccagattctgaaacaggccctctgtggcctgtctctattagctgggttccgggaggcagagaggagtgaccgggcactggcactgcgatcaggaagactggacccccagcccccagggcccccctccccccacttagtgctggtcctaggtcctctgaggcactcatctactgaatgacctctctacttccccttcttgccattattaacccatttttgtttattttccttaaatttttagccatttctccatgggccaccgcccagctcatgtaggtgagcctgggcagcttctgttggcagagcttttgcatttcctgtgtttgtcctgggttctggggcatcagccagctaccccttgtgggcaaaggcagggccacttttgaagtcttccctcagatttccattgtgtggcctggtgggtcagggggagtctttgcaccaaagatgtcctgactttgcccccttgcccatcagccatttgccatcaccccaaacaactcagcttcggggccggtgaggggaggggcctcccccagcacagatgaggagcagctggggtaggctgtctgtgccatggccccccactcccccttcccttggagggagaggtggcaggaatacttcacctttcctctccctcaggggcaggtggtggaggggcgcccagggtcgtctttgtgtatgggggaaggcgctgggtgcctgcagcgcctcccttgtctcagatggtgtgtccagcactcgattgttgtaaactgttgttttgtatgagcgaaattgtctttactaaacagatttaatagttaaaaaaaaaaaaaaaaaaa

SEQ ID NO: 24 - protein, 370 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKGCLHDKNSDQATWLTRL

SEQ ID NO: 25 - protein, 400 aa, Homo sapiens
MLSNSQGQSPPVPFPAPAPPPQPPTPALPHPPAQPPPPPPQQFPQFHVKSGLQIKKNAIIDDYKVTSQVLGLGINGKVLQIFNKRTQEKFALKMLQDCPKARREVELHWRASQCPHIVRIVDVYENLYAGRKCLLIVMECLDGGELFSRIQDRGDQAFTEREASEIMKSIGEAIQYLHSINIAHRDVKPENLLYTSKRPNAILKLTDFGFAKETTSHNSLTTPCYTPYYVAPEVLGPEKYDKSCDMWSLGVIMYILLCGYPPFYSNHGLAISPGMKTRIRMGQYEFPNPEWSEVSEEVKMLIRNLLKTEPTQRMTITEFMNHPWIMQSTKVPQTPLHTSRVLKEDKERWEDVKEEMTSALATMRVDYEQIKIKKIEDASNPLLLKRRKKARALEAAALAH

SEQ ID NO: 26 - protein, 16 aa, Artificial Sequence
  Sequence from the homeodomain of the Antennapedia protein
RQIKIWFQNRRMKWKK

SEQ ID NO: 27 - DNA, 20 bp, Artificial Sequence
  Forward primer for Gadd45a
gatgccctggaggaagtgct

SEQ ID NO: 28 - DNA, 20 bp, Artificial Sequence
  Reverse primer for Gadd45a
agcaggcacaacaccacgtt

SEQ ID NO: 29 - DNA, 20 bp, Artificial Sequence
  Forward primer for GAPDH
tgcaccaccaactgcttagc

SEQ ID NO: 30 - DNA, 21 bp, Artificial Sequence
  Reverse primer for GAPDH
ggcatggactgtggtcatgag

SEQ ID NO: 31 - protein, 580 aa, Homo sapiens
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 32 - protein, 601 aa, Homo sapiens
MEVPQPEPAPGSALSPAGVCGGAQRPGHLPGLLLGSHGLLGSPVRAAASSPVTTLTQTMHDLAGLGSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENVRFWKAGVGALREEEGACWGGSLACEDPPLPSWLQDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 33 - protein, 490 aa, Homo sapiens
MSEEARFCFSPGENSKTMSTELFSSTREEGSSGSGPSFRSNQRKMLNLLLERDTSFTVCPDVPRTPVGKFLGDSANLSILSGGTPKRCLDLSNLSSGEITATQLTTSADLDETGHLDSSGLQEVHLAGMNHDQHLMKCSPAQLLCSTPNGLDRGHRKRDAMCSSSANKENDNGNLVDSEMKYLGSPITTVPKLDKNPNLGEDQAEEISDELMEFSLKDQEAKVSRSGLYRSPSMPENLNRPRLKQVEKFKDNTIPDKVKKKYFSGQGKLRKGLCLKKTVSLCDITITQMLEEDSNQGHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 34 - protein, 473 aa, Homo sapiens
MSTELFSSTREEGSSGSGPSFRSNQRKMLNLLLERDTSFTVCPDVPRTPVGKFLGDSANLSILSGGTPKRCLDLSNLSSGEITATQLTTSADLDETGHLDSSGLQEVHLAGMNHDQHLMKCSPAQLLCSTPNGLDRGHRKRDAMCSSSANKENDNGNLVDSEMKYLGSPITTVPKLDKNPNLGEDQAEEISDELMEFSLKDQEAKVSRSGLYRSPSMPENLNRPRLKQVEKFKDNTIPDKVKKKYFSGQGKLRKGLCLKKTVSLCDITITQMLEEDSNQGHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 35 - protein, 551 aa, Homo sapiens
MEGAAPCRVSLKVSLGQRCWERAWRFDLGMGVVSSLTSHPLQNIAESGRPQLEAHLETRGGGHVGRRFCFSPGENSKTMSTELFSSTREEGSSGSGPSFRSNQRKMLNLLLERDTSFTVCPDVPRTPVGKFLGDSANLSILSGGTPKRCLDLSNLSSGEITATQLTTSADLDETGHLDSSGLQEVHLAGMNHDQHLMKCSPAQLLCSTPNGLDRGHRKRDAMCSSSANKENDNGNLVDSEMKYLGSPITTVPKLDKNPNLGEDQAEEISDELMEFSLKDQEAKVSRSGLYRSPSMPENLNRPRLKQVEKFKDNTIPDKVKKKYFSGQGKLRKGLCLKKTVSLCDITITQMLEEDSNQGHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 36 - protein, 400 aa, Homo sapiens
MSTELFSSTREEGSSGSGPSFRSNQRKMLNLLLERDTSFTVCPDVPRTPVGKFLGDSANLSILSGSPGFFRTSGSAFSWDDNGNLVDSEMKYLGSPITTVPKLDKNPNLGEDQAEEISDELMEFSLKDQEAKVSRSGLYRSPSMPENLNRPRLKQVEKFKDNTIPDKVKKKYFSGQGKLRKGLCLKKTVSLCDITITQMLEEDSNQGHLIGDFSKVCALPTVSGKHQDLKYVNPETVAALLSGKFQGLIEKFYVIDCRYPYEYLGGHIQGALNLYSQEELFNFFLKKPIVPLDTQKRIIIVFHCEFSSERGPRMCRCLREEDRSLNQYPALYYPELYILKGGYRDFFPEYMELCEPQSYCPMHHQDHKTELLRCRSQSKVQEGERQLREQIALLVKDMSP

SEQ ID NO: 37 - protein, 166 aa, Homo sapiens [sequence omitted]

SEQ ID NO: 38 - protein, 473 aa, Homo sapiens; identical to SEQ ID NO: 34

SEQ ID NO: 39 - protein, 473 aa, Homo sapiens; identical to SEQ ID NO: 34

SEQ ID NO: 40 - protein, 131 aa, Homo sapiens
MTLEEFSAGEQKTESDPDNVVLCLLAADEDDDRDVALQIHFTLIQAFCCENDINILRVSNPGRLAELLLLETDAGPAASEGAEQPPDLHCVLVTNPHSSQWKDPALSQLICFCRESRYMDQWVPVINLPER

SEQ ID NO: 41 - protein, 165 aa, Homo sapiens
MTLEEFSAGEQKTERMDKVGDALEEVLSKALSQRTITVGVYEAAKLLNVDPDNVVLCLLAADEDDDRDVALQIHFTLIQAFCCENDINILRVSNPGRLAELLLLETDAGPAASEGAEQPPDLHCVLVTNPHSSQWKDPALSQLICFCRESRYMDQWVPVINLPER

SEQ ID NO: 42 - protein, 131 aa, Homo sapiens; identical to SEQ ID NO: 40

SEQ ID NO: 43 - protein, 165 aa, Homo sapiens; identical to SEQ ID NO: 41

SEQ ID NO: 44 - protein, 165 aa, Homo sapiens; identical to SEQ ID NO: 41

SEQ ID NO: 45 - protein, 165 aa, Homo sapiens; identical to SEQ ID NO: 41

SEQ ID NO: 46 - protein, 165 aa, Homo sapiens; identical to SEQ ID NO: 41

SEQ ID NO: 47 - protein, 60 aa, Homo sapiens
MTLEEFSAGEQKTERMDKVGDALEEVLSKALSQRTITVGVYEAAKLLNVIHIHLNGRILP

SEQ ID NO: 48 - protein, 131 aa, Homo sapiens; identical to SEQ ID NO: 40

SEQ ID NO: 49 - protein, 165 aa, Homo sapiens; identical to SEQ ID NO: 41

SEQ ID NO: 50 - protein, 160 aa, Homo sapiens
MTLEELVACDNAAQKMQTVTAAVEELLVAAQRQDRLTVGVYESAKLMNVDPDSVVLCLLAIDEEEEDDIALQIHFTLIQSFCCDNDINIVRVSGMQRLAQLLGEPAETQGTTEARDLHCLLVTNPHTDAWKSHGLVEVASYCEESRGNNQWVPYISLQER

SEQ ID NO: 51 - protein, 205 aa, Homo sapiens
MTERRVPFSLLRGPSWDPFRDWYPHSRLFDQAFGLPRLPEEWSQWLGGSSWPGYVRPLPPAAIESPAVAAPAYSRALSRQLSSGVSEIRHTADRWRVSLDVNHFAPDELTVKTKDGVVEITGKHEERQDEHGYISRCFTRKYTLPPGVDPTQVSSSLSPEGTLTVEAPMPKLATQSNEITIPVTFESRAQLGGPEAAKSDETAAK

SEQ ID NO: 52 - protein, 205 aa, Homo sapiens [sequence omitted]

SEQ ID NO: 53 - protein, 205 aa, Homo sapiens; identical to SEQ ID NO: 51

SEQ ID NO: 54 - protein, 205 aa, Homo sapiens; identical to SEQ ID NO: 51

SEQ ID NO: 55 - protein, 205 aa, Homo sapiens; identical to SEQ ID NO: 51

SEQ ID NO: 56 - protein, 205 aa, Homo sapiens; identical to SEQ ID NO: 51

SEQ ID NO: 57 - protein, 305 aa, Homo sapiens
MENSQLCKLFIGGLNVQTSESGLRGHFEAFGTLTDCVVVVNPQTKRSRCFGFVTYSNVEEADAAMAASPHAVDGNTVELKRAVSREDSARPGAHAKVKKLFVGGLKGDVAEGDLIEHFSQFGTVEKAEIIADKQSGKKRGFGFVYFQNHDAADKAAVVKFHPIQGHRVEVKKAVPKEDIYSGGGGGGSRSSRGGRGGRGRGGGRDQNGLSKGGGGGYNSYGGYGGGGGGGYNAYGGGGGGSSYGGSDYGNGFGGFGSYSQHQSSYGPMKSGGGGGGGGSSWGGRSNSGPYRGGYGGGGGYGGSSF

SEQ ID NO: 58 - protein, 305 aa, Homo sapiens; identical to SEQ ID NO: 57

SEQ ID NO: 59 - protein, 305 aa, Homo sapiens; identical to SEQ ID NO: 57

SEQ ID NO: 60 - protein, 305 aa, Homo sapiens; identical to SEQ ID NO: 57

SEQ ID NO: 61 - protein, 305 aa, Homo sapiens; identical to SEQ ID NO: 57

SEQ ID NO: 62 - protein, 305 aa, Homo sapiens; identical to SEQ ID NO: 57

SEQ ID NO: 63 - protein, 305 aa, Homo sapiens; identical to SEQ ID NO: 57

SEQ ID NO: 64 - protein, 305 aa, Homo sapiens; identical to SEQ ID NO: 57

SEQ ID NO: 65 - protein, 305 aa, Homo sapiens; identical to SEQ ID NO: 57

SEQ ID NO: 66 - protein, 305 aa, Homo sapiens; identical to SEQ ID NO: 57

SEQ ID NO: 67 - protein, 305 aa, Homo sapiens; identical to SEQ ID NO: 57

SEQ ID NO: 68 - protein, 390 aa, Homo sapiens
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVHVCACPGRDRRTEEENLRKKGEPHHELPPGSTKRALSNNTSSSPQPKKKPLDGEYFTLQIRGRERFEMFRELNEALELKDAQAGKEPGGSRAHSSHLKSKKGQSTSRHKKLMFKTEGP

SEQ ID NO: 69 - protein, 390 aa, Homo sapiens
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGP

SEQ ID NO: 70 - protein, 390 aa, Homo sapiens
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGP

SEQ ID NO: 71 - protein, 393 aa, Homo sapiens [sequence omitted]

SEQ ID NO: 72 - protein, 393 aa, Homo sapiens [sequence omitted]

SEQ ID NO: 73 - protein, 393 aa, Homo sapiens [sequence omitted]

SEQ ID NO: 74 - protein, 393 aa, Homo sapiens [sequence omitted]

SEQ ID NO: 75 - protein, 160 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFS

SEQ ID NO: 76 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 77 - protein, 160 aa, Homo sapiens
MSEPAGDVRQNPCGSKACRRLFGPVDSEQLRRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFS

SEQ ID NO: 78 - protein, 164 aa, Homo sapiens; identical to SEQ ID NO: 76

SEQ ID NO: 79 - protein, 164 aa, Homo sapiens [sequence omitted]

SEQ ID NO: 80 - protein, 164 aa, Homo sapiens; identical to SEQ ID NO: 76

SEQ ID NO: 81 - protein, 163 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRK

SEQ ID NO: 82 - protein, 164 aa, Homo sapiens; identical to SEQ ID NO: 76

SEQ ID NO: 83 - protein, 639 aa, Homo sapiens
MEIIRSNFKSNLHKVYQAIEEADFFAIDGEFSGISDGPSVSALTNGFDTPEERYQKLKKHSMDFLLFQFGLCTFKYDYTDSKYITKSFNFYVFPKPFNRSSPDVKFVCQSSSIDFLASQGFDFNKVFRNGIPYLNQEEERQLREQYDEKRSQANGAGALSYVSPNTSKCPVTIPEDQKKFIDQVVEKIEDLLQSEENKNLDLEPCTGFQRKLIYQTLSWKYPKGIHVETLETEKKERYIVISKVDEEERKRREQQKHAKEQEELNDAVGFSRVIHAIANSGKLVIGHNMLLDVMHTVHQFYCPLPADLSEFKEMTTCVFPRLLDTKLMASTQPFKDIINNTSLAELEKRLKETPFNPPKVESAEGFPSYDTASEQLHEAGYDAYITGLCFISMANYLGSFLSPPKIHVSARSKLIEPFFNKLFLMRVMDIPYLNLEGPDLQPKRDHVLHVTFPKEWKTSDLYQLFSAFGNIQISWIDDTSAFVSLSQPEQVKIAVNTSKYAESYRIQTYAEYMGRKQEEKQIKRKWTEDSWKEADSKRLNPQCIPYTLQNHYYRNNSFTAPSTVGKRNLSPSQEEAGLEDGVSGEISDTELEQTDSCAEPLSEGRKKAKKLKRMKKELSPAGSISKNSPATLFEVPDTW

SEQ ID NO: 84 - protein, 639 aa, Homo sapiens; identical to SEQ ID NO: 83

SEQ ID NO: 85 - protein, 639 aa, Homo sapiens; identical to SEQ ID NO: 83

SEQ ID NO: 86 - protein, 639 aa, Homo sapiens; identical to SEQ ID NO: 83

SEQ ID NO: 87 - protein, 375 aa, Homo sapiens
MMEDDGQPRTLYVGNLSRDVTEVLILQLFSQIGPCKSCKMITEHTSNDPYCFVEFYEHRDAAAALAAMNGRKILGKEVKVNWATTPSSQKKDTSNHFHVFVGDLSPEITTEDIKSAFAPFGKISDARVVKDMATGKSKGYGFVSFYNKLDAENAIVHMGGQWLGGRQIRTNWATRKPPAPKSTQENNTKQLRFEDVVNQSSPKNCTVYCGGIASGLTDQLMRQTFSPFGQIMEIRVFPEKGYSFVRFSTHESAAHAIVSVNGTTIEGHVVKCYWGKESPDMTKNFQQVDYSQWGQWSQVYGNPQQYGQYMANGWQVPPYGVYGQPWNQQGFGVDQSPSAAWMGGFGAQPPQGQAPPPVIPPPNQAGYGMASYQTQ

SEQ ID NO: 88 - protein, 392 aa, Homo sapiens
MMEDDGQPRTLYVGNLSRDVTEVLILQLFSQIGPCKSCKMITEQPDSRRVNSSVGFSVLQHTSNDPYCFVEFYEHRDAAAALAAMNGRKILGKEVKVNWATTPSSQKKDTSNHFHVFVGDLSPEITTEDIKSAFAPFGKISDARVVKDMATGKSKGYGFVSFYNKLDAENAIVHMGGQWLGGRQIRTNWATRKPPAPKSTQENNTKQLRFEDVVNQSSPKNCTVYCGGIASGLTDQLMRQTFSPFGQIMEIRVFPEKGYSFVRFSTHESAAHAIVSVNGTTIEGHVVKCYWGKESPDMTKNFQQVDYSQWGQWSQVYGNPQQYGQYMANGWQVPPYGVYGQPWNQQGFGVDQSPSAAWMGGFGAQPPQGQAPPPVIPPPNQAGYGMASYQTQ

SEQ ID NO: 89 - protein, 375 aa, Homo sapiens; identical to SEQ ID NO: 87